(19) United States
(12) Patent Application Publication
(10) Pub. No.: US 2010/0204096 A1
(43) Pub. Date: Aug. 12, 2010

(54) TREATMENT OF INFLAMMATION, DEMYELINATION AND NEURONAL/AXONAL LOSS

(75) Inventors: Jacqueline A. Shukaliak-Quandt, Rockville, MD (US); John M. Hallenbeck, Kensington, MD (US); Hideaki Wakita, Rockville, MD (US)

Correspondence Address: KLARQUIST SPARKMAN, LLP (OTT-NIH), 121 S.W. SALMON STREET, SUITE #1600, PORTLAND, OR, 97204-2988, US

(73) Assignee: GOVERNMENT OF THE UNITED STATES OF AMERICAS AS REP, ROCKVILLE, MD (US)

(21) Appl. No.: 12/444,854

(22) PCT Filed: Oct. 9, 2007
(86) PCT No.: PCT/US07/21682

§ 371 Date: Feb. 25, 2010

Related U.S. Application Data
(60) Provisional application No. 60/828,732, filed on Oct. 9, 2006.
(60) Provisional application No. 60/905,741, filed on Mar. 8, 2007.

(51) Int. Cl. A61K 38/17; A61P 9/10; A61P 29/00; A61P 37/00

(52) U.S. Cl. 514/12

(57) ABSTRACT

The invention relates to compositions and methods for treating or preventing inflammation in a mammal comprising mucosal administration of an effective amount of an E-selectin polypeptide. Such compositions and methods are useful for treating or preventing a variety of conditions and diseases, including, for example, stroke, vascular dementia and autoimmune or inflammatory demyelinating diseases such as multiple sclerosis.

### CROSS-REFERENCE TO RELATED APPLICATIONS

This application claims priority to U.S. Provisional Application Ser. No. 60/828,732, filed Oct. 9, 2006 and to U.S. Provisional Application Ser. No. 60/905,741, filed Mar. 8, 2007, the contents of which applications are incorporated herein in their entireties.

This application is also related to U.S. Provisional Application Ser. No. 60/712,359, filed Aug. 30, 2005, U.S. application Ser. No. 10/296,423, filed Jun. 11, 2003, as well as to PCT application PCT/US01/16583, filed May 23, 2001, and to U.S. Provisional Application Ser. No. 60/206,693, filed May 24, 2000, the contents of which applications are incorporated herein in their entireties.

### STATEMENT REGARDING FEDERALLY FUNDED RESEARCH

The invention described herein was developed with support from the National Institutes of Health. The U.S. Government has certain rights in the invention.

### PARTIES TO A JOINT RESEARCH AGREEMENT

Not Applicable.

### BACKGROUND OF THE INVENTION

Adhesion molecules are cell surface glycoproteins which can be divided into four main groups: the integrin family, the immunoglobulin superfamily, the selectins, and the cadherins. Integrins are cell-surface receptors that mediate adhesion to the extracellular matrix (ECM) and cell-cell interactions. Most cells express several integrins that recognize a range of cell surface- and ECM-associated ligands. Individual integrins often bind more than one ligand, and they play multiple roles in differentiation and cell communication. Integrins are heterodimers of alpha and beta subunits. In mammals, at least 20 integrin heterodimers are known, where these 20 integrin heterodimers are comprised of 14 different types of alpha subunits and 8 types of beta subunits. A single beta chain can interact with multiple alpha chains forming integrins that bind different ligands. Some of the subunits are expressed exclusively on one type of cells, for example: beta 2 on leukocytes; lymphocytes can interact via integrins with proteoglycans and glycosaminoglycans which are the major components of ECM. Such interactions are important for lymphocyte migration, recognition, activation and differentiation. Some integrins may require activation in order to bind their ligand and anchor the cell to the ECM or to another cell.

The Ig superfamily members contain I-domains. X-ray crystallography revealed that I-domain consists of central core of 5 parallel and one short antiparallel beta-strands surrounded at the outer surface by 7 alpha-helices and loop regions. It contains about 200 amino acid residues. Each I-domain has a disulphide bridge. One of the member of the Ig superfamily is CD2, a glycoprotein receptor of T cells. It mediates cell-cell adhesion by binding to the surface molecule LFA-3 (from the same group) present on many cells including APC (antigen presenting cells). CD2-LFA-3 adhesion plays an important role in facilitating TCR/CD3 recognition of antigens presented by the MHC class II. CD2 is also required for T cell activation.

Selectins are membrane-attached mosaic proteins recognizing carbohydrates. They consist of a small cytosolic domain, a single transmembrane helix, an EGF-domain and an N-terminal lectin domain. Due to the selectins, white blood cells can migrate from blood vessels to the tissues. White blood cells adhere selectively to the walls of high endothelial venules. This adhesion is mediated by the L-selectins (homing receptors) on lymphocytes. Thus, the L-selectins determine the endothelial cells to which a lymphocytes will adhere. In contrast to the homing receptor, the two other selectins (P and E) are found on endothelial cells. E-selectin is only expressed when endothelial cells are actively attracting leukocytes.

When a tissue is infected, it secretes cytokines (IL-1, TNF) that stimulate endothelial cells in the venules to express P and E selectins. The white blood cells adhere to these selectins because their carbohydrate coats contain complementary structures.

P and E selectins appear on endothelial cells at different times and recruit different types of white blood cells. P-selectins are expressed earlier and draw leukocytes that act during the earliest phases of the immunologic defense. E-selectins (ELAM-1) are synthesized only when they are required and are involved in recruiting leukocytes such as neutrophils and macrophages to the inflammation sites. In some diseases white blood cells enter the tissues where they do not belong and cause their damage. For example, the inflammation of rheumatoid arthritis occurs when leukocytes enter the joints and release enzymes, oxygen radicals and other toxic factors. Cell-adhesion molecules may play a role in other diseases such as the spread of the cancer cells throughout the body.

Cadherins are integral transmembrane glycoproteins which mediate Ca⁺⁺-dependent cell-cell adhesion among most tissues, which is crucial for the mutual association of vertebrate cells. Cadherins are associated with the actin cytoskeleton through the cytoplasmic proteins, catenins.

There are reports that the presence or activation of certain adhesion molecules is associated with an increase in the risk of certain diseases, e.g., cardiovascular diseases, and undesirable inflammation is associated with a number of disorders.

Thus, there is a need for preventing or inhibiting certain disorders associated with cell activation and inflammation.

### SUMMARY OF THE INVENTION

The invention provides a method for preventing, inhibiting or treating inflammation associated with a variety of diseases, such as stroke, vascular dementia, autoimmune disorders (e.g., multiple sclerosis) and the like. As described hereinbelow, E-selectin instilled into the nares results in modulation of immune responses. While not meaning to limit the invention in any way, such modulation of immune responses may occur via lymphocytes that become regulatory T cells and that can suppress inflammation and immune responses. A variety of immune responses can be prevented, reduced, modified, inhibited or treated, including, for example, vascular inflammation giving rise to vascular occlusion, inflammation associated with demyelination and axonal/neuronal loss (e.g., in the brain or spinal cord), or inflammation associated with T cell mediated autoimmune disorders. Lymphocytes that have become regulatory T cells tend to migrate to the location of inflammation and, in that location within the body, the regulatory T cells release signaling molecules called cytokines. These cytokines suppress inflammation and immune responses.

E-selectin is a molecule that is only expressed on the lining of blood vessels when those blood vessels are becoming activated, which is proinflammatory and supportive of a local immune response. Thus, the anti-inflammatory and immunosuppressive effect of E-selectin administration is targeted to blood vessels that are activated and areas of immune activation where E-selectin is expressed or shed. By suppressing this activation through E-selectin administration, local inflammation and immune responses are suppressed. Immunomodulation by nasal or mucosal E-selectin administration is effective in immune-mediated inflammatory conditions that are mediated by proinflammatory T cells and other immune cell populations including monocytes, dendritic cells, microglia, Langerhans cells and others. Immunomodulation by nasal or mucosal E-selectin administration is effective for treating, reducing and/or inhibiting conditions including stroke, vascular dementia, multiple sclerosis (MS), rheumatoid arthritis, general vasculitis, and systemic lupus erythramatosus (SLE), as well as conditions with brain inflammation, e.g., MS, polyneuropathy, myasthenia gravis (MG) and Guillian Barr syndrome.

As described hereinbelow, when animals are administered E-selectin, they become resistant to stroke, vascular dementia, and experimental autoimmune encephalitis (EAE). EAE is a nonhuman animal form of the human disease, multiple sclerosis (MS). MS is an inflammatory demyelinating disease that leads to damage of nerve fibers in the brain and spinal cord and a resultant loss of brain and spinal cord function (e.g., weakness, spasticity, loss of vision, lack of coordination, paralysis, problems with bowel and bladder, etc.).

The invention thus provides methods and compositions for inhibiting or treating symptoms and resultant outcomes of inflammation or inflammation associated with T cell mediated autoimmune disorders. Surprisingly, the inventors have discovered that inhibiting inflammation of vascular tissues and/or nerve fibers in the brain and spinal cord can be accomplished by administering E-selectin, a cell adhesion molecule that mediates the adhesion of various leukocytes, including neutrophils, monocytes, eosinophils, NK cells, and a subset of T cells, to activated endothelium. E-selectin administration can also reduce local inflammation and tissue damage associated with a T cell mediated autoimmune disorder. Thus, because E-selectin is specifically expressed in vasculature that is activated (e.g., by injury or disruption related to disease), an immunosuppressive response is targeted to the site of activation. E-selectin expression in mammals previously treated with E-selectin leads to the release of immune system suppressive cytokines, which are released in response to endothelial activation.

Also provided is a method for preventing, inhibiting, reducing or treating inflammation and/or autoimmune diseases in a mammal. Such inflammation and/or autoimmune diseases can give rise to inflammatory occlusion of blood vessels and/or demyelination of nervous tissues in a mammal. The method includes mucosally administering an effective amount of E-selectin to the mammal. In some embodiments, the invention involves methods and compositions for preventing and treating stroke, vascular dementia, multiple sclerosis and other inflammatory and/or autoimmune diseases.

Thus, one aspect of the invention is a pharmaceutical composition comprising a pharmaceutically acceptable carrier and an effective amount of E-selectin. The E-selectin is an E-selectin polypeptide. Such an E-selectin polypeptide can be a mammalian E-selectin polypeptide, for example, a human E-selectin, a bovine E-selectin, a murine E-selectin, a rat E-selectin or any other E-selectin polypeptide from a mammalian source. Examples of E-selectin that can be used in the invention include E-selectin polypeptides with SEQ ID NO:5-8, 18, 19 or a combination thereof. The composition can be formulated for mucosal administration of E-selectin. For example, the mucosal administration can be intranasal, oral, enteral, vaginal, rectal, or respiratory administration. In some embodiments, the formulation is formulated for intra nasal administration, for example, as an aerosol. The aerosol can be a dry aerosol. Alternatively, the aerosol can be an atomized aqueous solution.

The pharmaceutical formulation of the invention is typically administered in an effective amount (e.g., a therapeutically effective amount). Such an effective amount of E-selectin is generally sufficient to reduce inflammation, to reduce demyelination, reduce axonal and/or neuronal loss, and/or to induce tolerance to E-selectin in a mammal. In some embodiments, an effective amount of E-selectin is sufficient to promote bystander-effect tolerance to E-selectin in a mammal. Examples of effective amounts of E-selectin include ranges of E-selectin of about 0.05 μg to about 500 mg or about 0.1 μg to about 100 mg. Another example of a desirable effective amount of E-selectin is a range of E-selectin of about 0.5 μg to about 50 mg.

Another aspect of the invention is a method for treating or preventing localized vascular inflammation in a mammal by administration to the mammal of an effective amount of E-selectin polypeptide. The E-selectin is an E-selectin polypeptide. Such an E-selectin polypeptide can be a mammalian E-selectin polypeptide, for example, a human E-selectin, a bovine E-selectin, a murine E-selectin, a rat E-selectin or any other E-selectin polypeptide from a mammalian source. Examples of E-selectin that can be used in the invention include E-selectin polypeptides with SEQ ID NO:5-8, 18, 19 or a combination thereof. Administration can be mucosal administration. For example, such mucosal administration of E-selectin can include nasal, oral, enteral, vaginal, rectal, or respiratory administration. In some embodiments, the administration is nasal or intranasal. The effective amount of E-selectin polypeptide is generally sufficient to induce bystander immune tolerance, or reduce localized inflammation, or reduce demyelination in the mammal. In some embodiments, the effective amount of E-selection is an amount effective for inducing expression of transforming growth factor beta (TGFβ), interleukin-4 (IL-4) and/or interleukin-10 (IL-10). The inventive methods can involve a series of separate E-selectin administrations. In some embodiments, the method involves a first series of administrations of E-selectin over a period of about two weeks. Such a first series of administrations can include about three to about seven administrations of E-selectin over the period of about two weeks. The method can further comprise at least one booster series of administrations of E-selectin after at least two weeks from the first series of administrations. In some embodiments, each booster series of administrations comprise about three to about seven administrations of E-selectin over the period of two weeks.

Another aspect of the invention is a method for inhibiting or treating inflammatory demyelination or inflammation associated with a T cell mediated autoimmune disorder in a mammal. The method includes administration of an amount of E-selectin polypeptide sufficient to induce bystander immune tolerance in the mammal, or reduce localized inflammation, or reduce demyelination in the mammal. In some embodiments, the effective amount of E-selection is an amount effective for inducing expression of transforming growth factor beta (TGFβ), interleukin-4 (IL-4) and/or interleukin-10 (IL-10). The administration can be to mucosal tissues, for example, to nasal tissues.

Another aspect of the invention is a method for treating or preventing stroke, vascular dementia or multiple sclerosis in a mammal comprising mucosal administration of an amount of E-selectin polypeptide sufficient to induce bystander immune tolerance in the mammal or reduce localized inflammation in the mammal. In some embodiments, the effective amount of E-selection is an amount effective for inducing expression of transforming growth factor beta (TGFβ), interleukin-4 (IL-4) and/or interleukin-10 (IL-10).

### DESCRIPTION OF THE FIGURES

FIG. 1A-B is the mucosal tolerance induction schedules employed for illustrative experiments described herein. For induction of mucosal tolerance, intranasal application of E-selectin was carried out. FIG. 1A shows the schedule for rats that received just a single series of E-selectin or PBS (control) administrations; rats receiving the single series of administrations are sometimes referred to herein as the “non-booster group.” FIG. 1B shows the schedule for rats that received a series of booster administrations of E-selectin or PBS (control) every three weeks; animals receiving such a series of administrations are sometimes referred to herein as the “booster group.”

FIG. 2 graphically illustrates the delayed type hypersensitivity (DTH) reaction in rats treated with E-selectin rats compared to rats treated with PBS. For this experiment, rats received an intranasal administration of E-selectin or phosphate buffered saline (PBS, control), and then were immunized with E-selectin in the footpad prior to challenge by an injection of E-selectin in the ear. This bar graph illustrates the change in thickness of the ear in animals that received intranasal instillation of E-selectin compared to the ear swelling in animals that received intranasal PBS. E-selectin administration on a single administration schedule significantly suppressed the delayed type hypersensitivity (DTH) induction of ear swelling in these animals. Therefore, intranasal instillation of E-selectin in the doses used in these animals does reduce or inhibit inflammation.

FIG. 3 graphically illustrates the discrimination indices of rats that received E-selectin and rats that did not receive E-selection for the object recognition test. Data for E-selectin treated animals is represented by diamond and * symbols. Data for animals that received PBS is represented by square symbols, and data for animals that were sham-operated is represented by triangle and + symbols. The discrimination indices of the E-selectin and sham groups were significantly increased as compared with the PBS group: * or +: p<0.05, ** or ++: p<0.01, *** or +++: p<0.001 by Fisher's protected least significant difference procedure, as compared to PBS-treated animals.

FIG. 4 graphically illustrates of the percent of alternation by rats on the T-maze spontaneous alternation test. Rats have an instinctive behavioral tendency to alternate their choices between the arms of the T-maze more often than they repeat their initial choice. In the E-selectin-treated animals, the percent of rats that alternated was significantly increased at 90 days, as compared with the PBS-treated animals. *: p<0.05 by χ² test.

FIG. 5A-C graphically illustrates the percentages of correct arm entrance on the T maze left/right discrimination memory retention test. Data for E-selectin treated animals is represented by diamond and * symbols. Data for animals that received PBS is represented by square symbols, and data for animals that were sham-operated is represented by triangle and + symbols. FIG. 5A shows the percentage of correct choices made by E-selectin treated, PBS control and sham operated animals two weeks after carotid artery ligation. E-selectin vs. PBS: p<0.0001 by repeated measure ANOVA. Sham vs PBS: p<0.0001 by repeated measure ANOVA. Sham vs. E-selectin: p=0.0040 by repeated ANOVA. FIG. 5B shows the percentage of correct choices made by animals treated with E-selectin, PBS control animals and sham operated animals six weeks after carotid artery ligation. E-selectin vs. PBS: p<0.0008 by repeated measure ANOVA. Sham vs PBS: p<0.0001 by repeated measure ANOVA. Sham vs. E-selectin: p=0.0030 by repeated ANOVA. Sham vs E-selectin: # p<0.05; ## p<0.01; ### p<0.001 by Fisher's PLSD post-hoc test. FIG. 5C shows the percentage of correct choices made by E-selectin treated animals, PBS control animals and sham operated animals ten weeks after carotid artery ligation. E-selectin vs. PBS: p<0.0003 by repeated measure ANOVA. Sham vs PBS: p<0.0026 by repeated measure ANOVA. Sham vs. E-selectin: p=0.6256 by repeated ANOVA. Some variability in the E-selectin group responses was observed at six weeks after ligation (FIG. 5B), but in general, by the third day after acquisition, the percentages of correct T-arm entries of the E-selectin treated and sham-operated animals were significantly increased as compared with the PBS group.

FIG. 6A-I shows photomicrographs of luxol fast blue stained sections of the corpus callosum (A, B, C), caudoputamen (D, E, F) and optic nerve (G, H, I) from rats that were subjected to a sham operation (C, F, I) or to bilateral ligation of the carotid arteries in animals that also received intranasal PBS (A, D, G) or E-selectin (B, E, H) on a booster administration schedule. Note that the extent of the white matter rarefaction was less severe in the E-selectin treated and sham-operated rats as compared with PBS group.

FIG. 7A-F shows photomicrographs of sections immunohistochemical stained using a cocktail of monoclonal antibodies directed against non-phosphorylated neurofilaments (SMI 311) that were obtained from the corpus callosum (A, B, C) and caudoputamen (D, E, F) from rats. The rats were subjected to a sham operation (C, F) or to bilateral ligation of the carotid arteries in animals that also received intranasal PBS (A, D) or E-selectin (B, E) on a booster administration schedule. Note that the extent of the white matter rarefaction was less severe in the E-selectin treated and sham-operated rats as compared with PBS group.

FIG. 8 graphically illustrates the fiber densities of the corpus callosum, caudoputamen and optic nerve in rats subjected to a sham operation or to bilateral ligation of the carotid arteries and intranasal administration of either PBS or E-selectin on a booster administration schedule. The fiber densities in E-selectin-treated animals were significantly higher than those in PBS-treated animals.

FIG. 9A-F shows photomicrographs of sections immunohistochemically stained for MHC class II antigens from the corpus callosum of rats that were subjected to a sham operation (C, F) or of rats that were subjected to bilateral ligation of the carotid arteries and intranasal PBS (A, D) or E-selectin (B, E) on a booster administration schedule. In E-selectin-treated rats, the number of microglia/macrophages positively immuno-labeled for MHC class II antigen in the white matter lesions were somewhat reduced in comparison to PBS-treated animals.

FIG. 10 shows histograms of the numerical densities of MHC class II immunopositive microglia/macrophages in the corpus callosum of rats subjected to a sham operation, or to bilateral ligation of the carotid arteries in animals that also received intranasal PBS or E-selectin on a booster administration schedule.

FIG. 11A-B illustrates that CD4 positive T cells infiltrate brain tissues after carotid artery occlusion. FIG. 11A-B show rat corpus callosum sections immunohistochemically stained for CD4 (a marker for T cells). Sham-treated rats exhibited little or no CD4 positive T cell infiltration (FIG. 11A). In contrast, after carotid artery occlusion, increased numbers of CD4 positive T cells were observed in the corpus callosum of rats (FIG. 11B).

FIG. 12A-F shows photomicrographs of immunohistochemically stained sections for detection of TNF-α in the corpus callosum. The rats were subjected to a sham operation (C, F) or to bilateral ligation of the carotid arteries in animals that also received intranasal PBS (A, D) or E-selectin (B, E) on a booster administration schedule. In the E-selectin-treated and sham-operated animals, TNF-immunopositive vessels were markedly less prominent than in PBS-treated animals.

FIG. 13 shows histograms of the numerical density of TNF-α-immunopositive vessels in the corpus callosum of rats subjected to a sham operation, or to bilateral ligation of the carotid arteries in animals that also received intranasal PBS or E-selectin on a booster administration schedule. In the E-selectin-treated and sham-operated animals, the number of TNF-α-immunopositive vessels was significantly reduced as compared with the PBS-treated animals.

FIG. 14A-F shows photomicrographs of sections immunohistochemically stained for detection of E-selectin in the corpus callosum. The rats were subjected to a sham operation (C, F) or to bilateral ligation of the carotid arteries in animals that also received intranasal PBS (A, D) or E-selectin (B, E) on a booster administration schedule. The sections were taken 90 days after carotid ligation. In the E-selectin-treated and sham-operated animals, E-selectin-immunopositive vessels were less prominent as compared to the PBS-treated animals.

FIG. 15 shows histograms of the numerical density of E-selectin-immunopositive vessels in the corpus callosum of rats subjected to a sham operation, or to bilateral ligation of the carotid arteries and either intranasal PBS or intranasal E-selectin on a booster administration schedule. In the E-selectin-treated and sham-operated animals, the number of E-selectin-immunopositive vessels was significantly reduced as compared with the PBS-treated animals.

FIG. 16 illustrates that occlusion of blood vessels feeding brain tissues leads to a number of problems, including disturbances in axonal transport, demyelination, induction of metalloproteinases (MMPs), blood brain barrier problems, activation of glial cells, infiltration of lymphocytes, edema, inflammation and immunological reactions that all lead to heightened tissue damage and further vascular injury.

FIG. 17 is a schematic of an exemplary immunization protocol.

FIG. 18 provides a comparison of E-selectin sequences where Line 1 is wild type E-Selectin (human), GenBank Acc. No. M30640 (SEQ ID NO:21); Line 2: wild type E-Selectin (human), GenBank Acc. No. NM_{—}000450 (SEQ ID NO:22); Line 3: “new” recombinant E-Selectin, no tags (SEQ ID NO:23). Line 4: “old” recombinant E-Selectin protein with c-myc, Histidine tags (SEQ ID NO:24). The Underlined sequences are signal peptide sequences; the symbol ****** indicates that the sequences are part of the transmembrane domain; the symbol ###### indicates that the sequences are c-myc and/or Histidine tags; the symbol ////// indicates that the sequences Lectin C-type domain sequences; the symbol @@@@@@ indicates that the sequences are Calcium binding EGF-like domain sequences. See, Nession et al., PNAS 87, 1673-1677 (1990); Zhang et al., FEMS Microbiol Lett 227, 303-309 (2003); Kiely et al., J Immunol 171, 3216-3224 (2003).

FIG. 19 is a comparison of human (SEQ ID NO:25, top sequence) and mouse (SEQ ID NO:26, bottom sequence) E-selectin sequences.

FIG. 20 shows that murine E-selectin reduces the severity of EAE.

### DETAILED DESCRIPTION OF THE INVENTION

The invention provides compositions and methods for treating and preventing negative consequences of inflammation.

### DEFINITIONS

As used herein, “tolerance” refers to an antigen-induced immune unresponsiveness in a mammal upon re-exposure of the mammal to the antigen. The antigen has previously been administered to induce such immune unresponsiveness. The induced immune unresponsiveness may be specific for the administered antigen or may be antigen-non-specific as a result of production of an antigen-non-specific suppressor substance such as transforming growth factor beta (TGFβ), interleukin-4 (IL-4) or interleukin-10 (IL-10).

As used herein “bystander tolerance” means that T-cells, which are primed by E-selectin administration, release immune system suppressive cytokines after subsequent stimulation by that antigen (E-selectin). Such suppressor T cells arise in the mucosal immune system and migrate to systemic sites where, upon antigen-specific reactivation, the suppressor T cells release TGFβ, IL-4, IL-10 and other suppressive cytokines

A delayed type hypersensitivity reaction as used herein is a measure of whether the immune system actively reacts to an antigen or whether the immune system exhibits tolerance towards the antigen, a reduced immune response or substantially no immune response to the antigen. An antigen is introduced intradermally, and after about 48-72 hours post-injection the site of intradermal administration is observed. If the immune system actively reacts to the antigen, the injection site will appear red, inflamed, thickened, and tender. The swelling and thickening of the skin are a result of an immune response. The lack of a delayed type hypersensitivity response to the antigen indicates that the immune system has a reduced immune response, substantially no immune response to the antigen or is tolerant of the antigen.

As used herein, a “subject” is a mammal or bird to which the E-selectin compositions of the invention are administered. Thus, the subject can be bovine, rat, mouse, dog, pig, horse, goat, monkey, ape, human or other domestic or zoo mammal. In addition, the subject can be chicken, turkey, parrot or other domestic or zoo bird.

E-Selectin

E-selectin (also known as ELAM-1, CD62, and CD62E) is a cytokine-inducible cell surface glycoprotein that is found on endothelial cells. E-selectin is expressed in vascular endothelial tissue (Pober et al., J. Immunol., 136:1680 (1986); Bevilacqua et al., Proc. Natl. Acad. Sci., 84:9238 (1987)), and is induced in response to the cytokines IL-1 and TNF, as well as bacterial lipopolysaccharide (LPS), through transcriptional up-regulation. (Pobor et al., supra; see also, Montgomery et al., Proc. Natl. Acad. Sci., 88:6523 (1991)). E-selectin is also a cell adhesion molecule that mediates the adhesion of various leukocytes, including neutrophils, monocytes, eosinophils, natural killer (NK) cells, and a subset of T cells, to activated endothelium (Bevilacqua et al, Science, 243:1160 (1989); Graber et al., J. Immunol., 145:819 (1990); Carlos et al., Blood, 77:2266 (1991); Hakkert et al., Blood, 78:2721 (1991); and Picker et al., Nature, 349:796 (1991)). Some workers hypothesize that activation of vascular endothelial cells is involved in inflammatory vascular tissue damage leading to thrombosis (Fareed et al., Clin. Lab. Med., 15:39 (1995)).

Structurally, E-selectin belongs to a family of adhesion molecules termed “selectins” that also includes P-selectin and L-selectin (see reviews in Lasky, Science, 258:964 (1992) and Bevilacqua and Nelson, J. Clin. Invest., 91:379 (1993)). These molecules are characterized by common structural features such as an amino-terminal lectin-like domain, an epidermal growth factor (EGF) domain, and a discrete number of complement repeat modules (approximately 60 amino acids each) similar to those found in certain complement binding proteins.

Examples of nucleic acid and amino acid sequences for different types and species of E-selectin can be found in the art, for example, in the NCBI database. See website at ncbi.nlm.nih.gov. Thus, for example, the NCBI database provides a human E-selectin precursor amino acid sequence as accession number P16581 (gi: 126180). This sequence is provided below for easy reference as SEQ ID NO:1.

| | |
|---|---|
| 1 | MIASQFLSAL TLVLLIKESG AWSYNTSTEA MTYDEASAYC |
| 41 | QQRYTHLVAI QNKEEIEYLN STLSYSPSYY WIGIRKVNNV |
| 81 | WVWVGTQKPL TEEAKNWAPG EPNNRQKDED CVEIYIKREK |
| 121 | DVGMWNDERC SKKKLALCYT AACTNTSCSG HGECVETINN |
| 161 | YTCKCDPCFS GLKCEQIVNC TALESPEHHS LVCSHPLGNF |
| 201 | SYNSSCSISC DRGYLPSSME TMQCMSSGEW SAPIPACNVV |
| 241 | ECDAVTNPAN GFVECFQNPG SFPWNTTCTF DCEECFELMG |
| 281 | AQSLQCTSSG NWDNEKPTCK AVTCRAVRQP QNGSVRCSHS |
| 321 | PAGEFTFKSS CNFTCEEGFM LQGPAQVECT TQGQWTQQIP |
| 361 | VCEAFQCTAL SNPERGYMNC LPSASGSFRY GSSCEFSCEQ |
| 401 | GFVLKGSKRL QCGPTCEWDN EKPTCEAVRC DAVHQPPKGL |
| 441 | VRCAHSPIGE FTYKSSCAFS CEEGFELHGS TQLECTSQGQ |
| 481 | WTEEVPSCQV VKCSSLAVPG KINMSCSGEP VFGTVCKFAC |
| 521 | PEGWTLNGSA ARTCGATGHW SGLLPTCEAP TESNIPLVAG |
| 561 | LSAAGLSLLT LAPFLLWLRK CLRKAKKFVP ASSCQSLESD |
| 601 | GSYQKPSYIL |

The mature sequence for this human E-selectin extends from about amino acid 22 to amino acid 610. The sequence for this mature E-selectin polypeptide is therefore as follows (SEQ ID NO:2).

| | |
|---|---|
| 22 | WSYNTSTEA MTYDEASAYC |
| 41 | QQRYTHLVAI QNKEEIEYLN SILSYSPSYY WIGIRKVNNV |
| 81 | WVWVGTQKPL TEEAKNWAPG EPNNRQKDED CVEIYIKREK |
| 121 | DVGMWNDERC SKKKLALCYT AACTNTSCSG HGECVETINN |
| 161 | YTCKCDPGFS GLKCEQIVNC TALESPEHGS LVCSHPLGNF |
| 201 | SYNSSCSISC DRGYLPSSME TMQCMSSGEW SAPIPACNVV |
| 241 | ECDAVTNPAN CFVECFQNPG SFPWNTTCTF DCEEGFELMG |
| 281 | AQSLQCTSSG NWDNEKPTCK AVTCRAVRQP QNGSVRCSHS |
| 321 | PAGEFTFKSS CNFTCEEGFM LQGPAQVECT TQGQWTQQIP |
| 361 | VCEAFQCTAL SNPERGYMNC LPSASGSFRY GSSCEFSCEQ |
| 401 | GFVLKGSKRL QCGPTGEWDN EKPTCEAVRC DAVHQPPKGL |
| 441 | VRCAHSPIGE FTYKSSCAFS CEEGFELHGS TQLECTSQGQ |
| 481 | WTEEVPSCQV VKCSSLAVPG KINMSCSGEP VFGTVCKFAC |
| 521 | PEGWTLNGSA ARTCGATGHW SGLLPTCEAP TESNIPLVAG |
| 561 | LSAAGLSLLT LAPFLLWLRK CLRKAKKFVP ASSCQSLESD |
| 601 | GSYQKPSYIL |

In some embodiments, an extracellular E-selectin domain may be administered to subjects. The extracellular domain of the human E-selectin provided above includes a sequence of about amino acid 22 to about amino acid 556 and therefore has the following sequence (SEQ ID NO:3).

| | |
|---|---|
| 22 | WSYNTSTEA MTYDEASAYC |
| 41 | QQRYTHLVAI QNKEEIEYLN SILSYSPSYY WIGIRKVNNV |
| 81 | WVWVGTQKPL TEEAKNWAPG EPNNRQKDED CVEIYIKREK |
| 121 | DVGMWNDERC SKKKLALCYT AACTNTSCSG HGECVETINN |
| 161 | YTCKCDPGFS GLKCEQIVNC TALESPEHGS LVCSRPLGNF |
| 201 | SYNSSCSISC DRGYLPSSME TMQCMSSGEW SAPIPACNVV |
| 241 | ECDAVTNPAN GFVECFQNPG SFPWNTTCTF DCEEGFELMG |
| 281 | AQSLQCTSSG NWDNEKPTCK AVTCRAVRQP QNGSVRCSHS |
| 321 | PAGEFTFKSS CNFTCEEGFM LQGPAQVECT TQGQWTQQIP |
| 361 | VCEAFQCTAL SNPERGYMNC LPSASGSFRY GSSCEFSCEQ |
| 401 | GFVLKGSKRL QCGPTGEWDN EKPTCEAVRC DAVHQPPKGL |
| 441 | VRCAHSPIGE FTYKSSCAFS CEEGFELHGS TQLECTSQGQ |
| 481 | WTEEVPSCQV VKCSSLAVPG KINMSCSGEP VFGTVCKFAC |
| 521 | PEGWTLNGSA ARTCGATGHW SGLLPTCEAP TESNIP |

In some embodiments human E-selectin may be administered to a subject. As is known to the skilled artisan, some sequence variation exists in human E-selectins. Thus, other human E-selectin amino acid sequences can be found in the NCBI database, for example, as accession numbers AAN01237 (gi: 22536178), CAA17434 (gi: 3115964), AAA52376 (gi: 537524), CA119357 (gi: 56417699), among others. According to the invention, any such human E-selectin polypeptides can be used for administration to a subject.

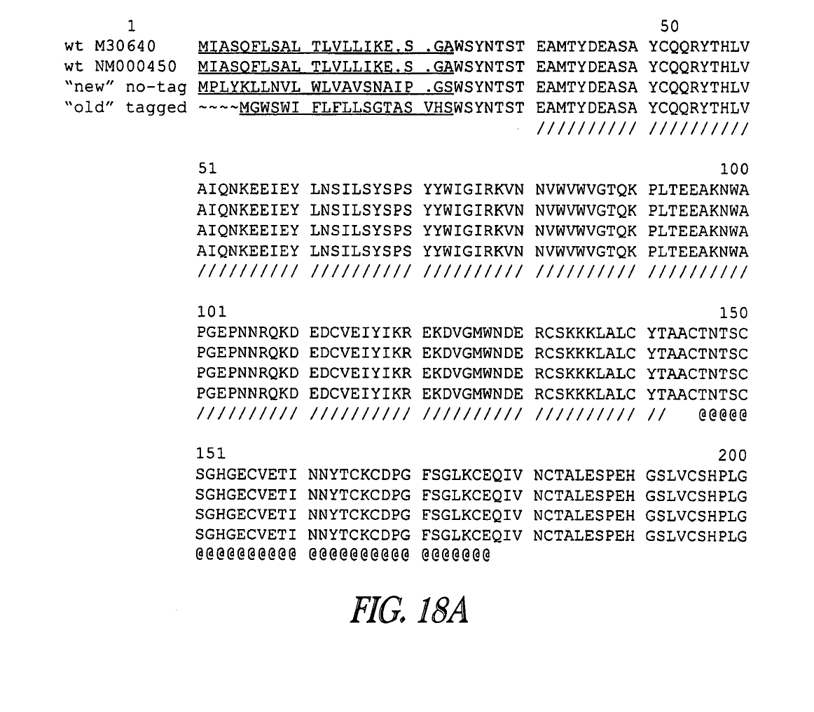

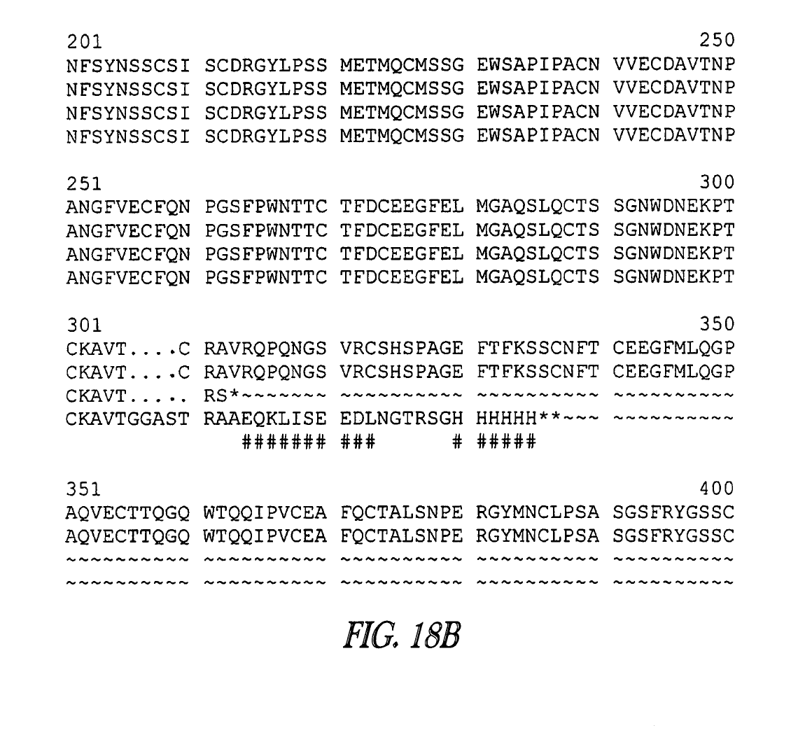

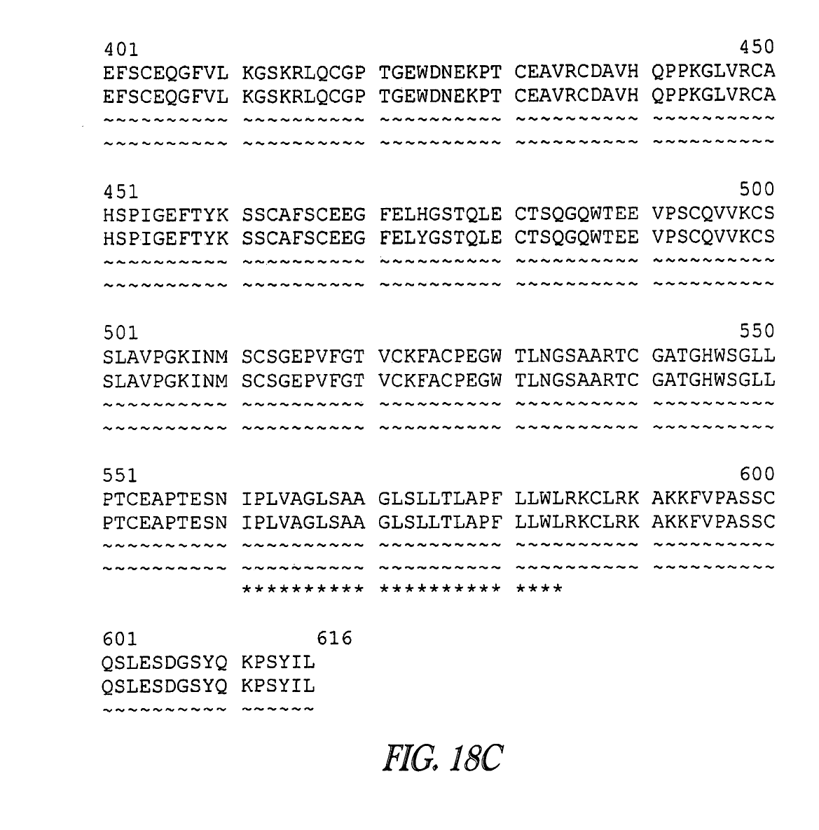

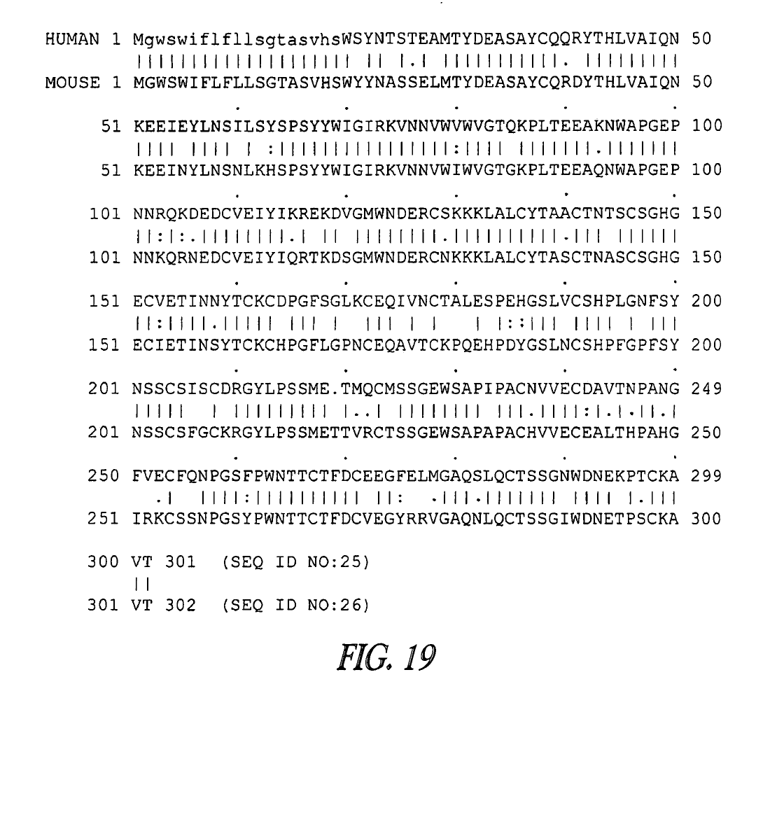

As indicated above, wild type E-selectins have a total about of 589 amino acids. Such wild type E-selectins include a lectin domain, an epidermal growth factor-like (EGF) domain, and a series of between 2 and 9 consensus repeat domains similar to those of complement proteins. Thus, wild type E-selectin, for example, the E-selectin sequences provided in FIG. 18, can generally include the structural elements shown below.

Amino acids 1-21: signal sequence

Amino acids 22-140: lectin like domain

Amino acid 144-175: EGF like domain

Amino acid 180-237: first consensus repeat domain

Amino acid 242-300: second consensus repeat domain

Amino acid: 300-363 third consensus repeat domain

Amino acid 367-426: fourth consensus repeat domain

Amino acid 430-489: fifth consensus repeat domain

Amino acid 493-548: sixth consensus repeat domain

A membrane spanning domain of about 22 amino acids and an intracellular domain of about 32 amino acids are also present at the carboxyl terminus of wild type E-selectin (see FIG. 18). However, neither the membrane-spanning domain nor the intracellular domain need be present in the E-selectins used in the compositions and methods of the invention. Moreover, several of the consensus repeat domains can be eliminated from the E-selectin used in the compositions and methods of the invention.

Thus, in some embodiments, the E-selectin is a soluble E-selectin that does not contain the membrane spanning domain or the intracellular domain. Soluble E-selectin can be generated by enzymatic cleavage (to eliminate the membrane spanning domain and/or the intracellular domain) or by recombinant expression of the soluble E-selectin portion of the molecule. The exact amino acid sequence of E-selectin can therefore vary depending on the cleavage site chosen for deleting the membrane spanning and/or the intracellular domains, or the C-terminus selected for making a recombinant soluble E-selectin. In addition, the number of complement-like consensus repeats can vary.

Thus, in some embodiments, the extracellular portion of the E-selectin molecule is used. Such an extracellular region of E-selectin can have up to about 550 amino acids or more desirably up to about 535 amino acids. However, in many embodiments the extracellular domain of E-selectin has less than about 550 to 535 amino acids. For example, the extracellular domain used in the compositions and methods of the invention can have about 1 to about 260 amino acids, or any integer in between, fewer amino acids than the 535-550 amino acids that generally comprises the E-selectin extracellular domain. Thus, the extracellular domain of E-selectin that is used in the compositions and methods of the invention can have at least about 275, about 280, about 285, about 290, about 295, about 300, about 310, about 315, about 320, about 325 amino acids or any integer from at least about 275 to at least about 325 amino acids.

In general, the extracellular domain of E-selectin includes, from the amino terminus of the E-selectin protein: the lectin domain, the epidermal growth factor-like (EGF) domain, and a series of between 2 and 9 consensus repeat domains similar to those of complement proteins. Thus, the E-selectin can have about 2, about 3, about 4, about 5, about 6, about 7, about 8 or about 9 consensus repeat domains. Depending on the number of consensus repeat domains, the total number of amino acids and the molecular weight of E-selectin will therefore change.

The consensus repeat domains of E-selectin are also called complement control protein (CCP) modules, short consensus repeats (SCRs) or SUSHI repeats. These consensus repeat domains contain approximately 60 amino acid residues and have been identified in several proteins of the complement system. For example, there are two consensus repeat domains at positions 13-53 and 57-112 in the following sequence (NCBI accession number AAQ67702; gi: 34420911; SEQ ID NO:4).

| | |
|---|---|
| 1 | PKGLVRCAHS PIGEFTYKSS CAFSCEEGFE LYGSTQLECT |
| 41 | SQGQWTEEVP SCQVVKCSSL AVPGKINMSC SGEPVFGTVC |
| 81 | KFACPEGWTL NGSAARTCGA TGHWSGLLPT CEAPTESNIP |
| 121 | LVAGLSAAGL SLLTLAPF |

In one embodiment, a human E-selectin protein is used in the compositions and methods of the invention that has about 306 amino acids (e.g. SEQ ID NO:5).

| | |
|---|---|
| 1 | MPLYKLLNVL WLVAVSNAIP GSWSYNTSTE AMTYDEASAY |
| 41 | CQQRYTHLVA IQNKEEIEYL NSILSYSPSY YWIGIRKVNN |
| 81 | VWVWVGTQKP LTEEAKNWAP GEPNNRQKDE DCVEIYIKRE |
| 121 | KDVGMWNDER CSKKKLALCY TAACTNTSCS GHGECVETIN |
| 161 | NYTCKCDPGF SGLKCEQIVN CTALESPEHG SLVCSHPLGN |
| 201 | FSYNSSCSIS CDRGYLPSSM ETMQCMSSGE WSAPIPACNV |
| 241 | VECDAVTNPA NGFVECFQNP GSFPWNTTCT FDCEEGFELM |
| 281 | GAQSLQCTSS GNWDNEKPTC KAVTRS |

The SEQ ID NO:5 E-selectin sequence is part of the third sequence identified as the “new” recombinant E-Selectin with no tags shown in FIG. 18. In another embodiment, the human E-selectin protein used in the compositions and methods of the invention that has about 304 amino acids (e.g. SEQ ID NO:6), because the C-terminal arginine and serine residues are not present.

| | |
|---|---|
| 1 | MPLYKLLNVL WLVAVSNAIP GSWSYNTSTE AMTYDEASAY |
| 41 | CQQRYTHLVA IQNKEEIEYL NSILSYSPSY YWIGIRKVNN |
| 81 | VWVWVGTQKP LTEEAKNWAP GEPNNRQKDE DCVEIYIKRE |
| 121 | KDVGMWNDER CSKKKLALCY TAACTNTSCS GHGECVETIN |
| 161 | NYTCKCDPGF SGLKCEQIVN CTALESPEHG SLVCSHPLGN |
| 201 | FSYNSSCSIS CDRGYLPSSM ETMQCMSSGE WSAPIPACNV |
| 241 | VECDAVTNPA NGFVECFQNP GSEPWNTTCT FDCEEGFELM |
| 281 | GAQSLQCTSS GNWDNEKPTC KAVT |

In another embodiment, a human E-selectin protein without a signal sequence is used in the compositions and methods of the invention that has about 284 amino acids (e.g. SEQ ID NO: 7).

| | |
|---|---|
| 1 | WSYNTSTEAM TYDEASAYCQ QRYTHLVAIQ NKEEIEYLNS |
| 41 | ILSYSPSYYW IGIRKVNNVW VWVGTQKPLT EEAKNWAPGE |
| 81 | PNNRQKDEDC VEIYIKREKD VGMWNDERCS KKKLALCYTA |
| 121 | ACTNTSCSGH GECVETINNY TCKCDPGFSG LKCEQIVNCT |
| 161 | ALESPEHGSL VCSHPLGNFS YNSSCSISCD RGYLPSSMET |
| 201 | MQCMSSGEWS APIPACNVVE CDAVTNPANG FVECFQNPGS |
| 241 | FPWNTTCTFD CEEGFELMGA QSLQCTSSGN WDNEKPTCKA |
| 281 | VTRS |

In a further embodiment, the human E-selectin protein used in the compositions and methods of the invention that has about 282 amino acids (e.g. SEQ ID NO:8), because the signal sequence and the C-terminal arginine and serine residues are not present.

| | |
|---|---|
| 1 | WSYNTSTEAM TYDEASAYCQ QRYTHLVAIQ NKEEIEYLNS |
| 41 | ILSYSPSYYW IGIRKVNNVW VWVGTQKPLT EEAKNWAPGE |
| 81 | PNNRQKDEDC VEIYIKREKD VGMWNDERCS KKKLALCYTA |
| 121 | ACTNTSCSGH GECVETINNY TCKCDPGFSG LKCEQIVNCT |
| 161 | ALESPEHGSL VCSHPLGNFS YNSSCSISCD RGYLPSSMET |
| 201 | MQCMSSGEWS APIPACNVVE CDAVTNPANG FVECFQNPGS |
| 241 | FPWNTTCTFD CEEGFELMGA QSLQCTSSGN WDNEKPTCKA |
| 281 | VT |

These approximate 282-284 amino acid sequences for E-selectin has the lectin domain, the EGF domain, and two complement-like consensus repeats.

In some embodiments, a signal sequence may be present on the N-terminus of the E-selectin. One example of a signal sequence that can be used is the MGWSWIFLFLLSGTASVHS (SEQ ID NO:27) signal sequence. Another example of a signal sequence that can be used is the MPLYKLLNVLWLVAVSNAI (SEQ ID NO:28) signal sequence. Also in some embodiments, a C-terminal tag sequence may be used with the E-selectin. One example of a C-terminal tag sequence that can be used is a histidine tag sequence, for example, the GGASTRAAEQKLI SEEDLNGTRSGHHHHHH (SEQ ID NO:29) tag sequence.

In addition, in some embodiments it may be useful to administer E-selectin from non-human species to the subject. Thus, for example, non-human E-selectin may optimally inhibit inflammation and/or induce tolerization to E-selectin in some human subjects. Therefore, the invention is directed to administering non-human E-selectin to subjects, and such non-human E-selectin can include just the extracellular portion of the E-selectin and/or the extracellular portion of E-selectin with just 2 to about 9 consensus repeat domains. Many sources and examples of non-human E-selectin are available. For example, nucleic acid and amino acid sequences for different types of non-human E-selectin can be found in the art, for example, in the NCBI database. See website at ncbi.nlm.nih.gov. Thus, for example, bovine, rat, mouse, dog, pig, horse, goat, monkey, ape or other mammalian E-selectin polypeptides can be administered to a subject. Sequences for such mammalian E-selectins are available, for example, in the NCBI database.

One example of a bovine E-selectin polypeptide sequence that can be found in the NCBI database is the bovine E-selectin sequence with accession number P98107 (gi: 1346435). This bovine E-selectin sequence is the precursor sequence and is provided below for easy reference (SEQ ID NO:9).

| | |
|---|---|
| 1 | MIVSQYLSAL TFVLLLFKES RTWSYHASTE MMTFEEARDY |
| 41 | CQKTYTALVA IQNQEEIEYL NSTFSYSPSY YWTGIRKING |
| 81 | TWTWIGTNKS LTKEATNWAP GEPNNKQSDE DCVEIYIKRE |
| 121 | KDSGKWNDEK CTKQKLALCY KAACNPTPCG SHGECVETIN |
| 161 | NYTCQCHPGF KGLKCEQVVT CPAQKHPEHG HLVCNPLGKF |
| 201 | TYNSSCSISC AEGYLPSSTE ATRCMSSGEW STPLPKCNVV |
| 241 | KCDALSNLDN GVVNCSPNHG SLPWNTTCTF ECQEGYKLTG |
| 281 | PQHLQCTSSG IWDNKQPTCK AVSCAAISHP QNGTVNCSHS |
| 321 | VVGDFAFKSS CHFTCAEGFT LQGPTQVECT AQGQWTQRVP |
| 361 | VCEVVRCSRL DVSGKLNMNC SCEPVLGTEC TFACPERWTL |
| 401 | NGSVVLTCGA TGHWSGMLPT CEAPTVSQTP LAVGLSTAGV |
| 441 | SLVTIPSFLF WLLKRLQKKA KKFSPASSCS SLKSNGCYST |
| 481 | PSKLI |

The mature sequence for this bovine E-selectin extends from about amino acid 23 to amino acid 485. The sequence for this mature bovine E-selectin polypeptide is therefore as follows (SEQ ID NO:10).

| | |
|---|---|
| 23 | WSYHASTE MMTFEEARDY |
| 41 | CQKTYTALVA IQNQEEIEYL NSTFSYSPSY YWIGIRKING |
| 81 | TWTWIGTNKS LTKEATNWAP GEPNNKQSDE DCVEIYIKRE |
| 121 | KDSGKWNDEK CTKQKLALCY KAACNPTPCG SHGECVETIN |
| 161 | NYTCQCHPGF KGLKCEQVVT CPAQKHPEHG HLVCNPLGKF |
| 201 | TYNSSCSISC AEGYLPSSTE ATRCMSSGEW STPLPKCNVV |
| 241 | KCDALSNLDN GVVNCSPNHG SLPWNTTCTF ECQEGYKLTG |
| 281 | PQHLQCTSSG IWDNKQPTCK AVSCAAISHP QNGTVNCSHS |
| 321 | VVGDFAFKSS CHFTCAEGFT LQGPTQVECT AQGQWTQRVP |
| 361 | VCEVVRCSRL DVSGKLNMNC SGEPVLGTEC TFACPERWTL |
| 401 | NGSVVLTCGA TGHWSGMLPT CEAPTVSQTP LAVGLSTAGV |
| 441 | SLVTIPSFLF WLLKRLQKKA KKFSPASSCS SLKSNGCYST |
| 481 | PSKLI |

An extracellular E-selectin domain may be used for tolerization of a subject. The extracellular domain of the bovine E-selectin provided above includes a sequence of about amino acid 23 to about amino acid 430 and therefore has the following sequence (SEQ ID NO:11).

| | |
|---|---|
| 23 | WSYHASTE MMTFEEARDY |
| 41 | CQKTYTALVA IQNQEEIEYL NSTFSYSPSY YWIGIRKING |
| 81 | TWTWIGTNKS LTKEATNWAP GEPNNKQSDE DCVEIYIKRE |
| 121 | KDSGKWNDEK CTKQKLALCY KAACNPTPCG SHGECVETIN |
| 161 | NYTCQCHPGF KGLKCEQVVT CPAQKHPEHG HLVCNPLGKF |
| 201 | TYNSSCSISC AECYLPSSTE ATRCMSSGEW STPLPKCNVV |
| 241 | KCDALSNLDN GVVNCSPNHG SLPWNTTCTF ECQEGYKLTG |
| 281 | PQHLQCTSSG IWDNKQPTCK AVSCAAISHP QNGTVNCSHS |
| 321 | VVGDFAFKSS CHFTCAEGFT LQGPTQVECT AQGQWTQRVP |
| 361 | VCEVVRCSRL DVSGKLNMNC SGEPVLGTEC TFACPERWTL |
| 401 | NGSVVLTCGA TGHWSGMLPT CEAPTVSQTP |

As is known to the skilled artisan, some sequence variation exists among bovine E-selectins. Thus, other bovine E-selectin amino acid sequences can be found in the NCBI database, for example, as accession numbers S36772 (gi: 480377) and NP 776606 (gi: 27806407), among others. According to the invention, any such bovine E-selectin polypeptides can be used for tolerization of a subject to E-selectin.

One example of a rat E-selectin polypeptide sequence that can be found in the NCBI database is the rat E-selectin sequence with accession number P98105 (gi: 1346437). This rat E-selectin sequence is the precursor sequence and is provided below for easy reference (SEQ ID NO:12).

| | |
|---|---|
| 1 | MNASCFLSAL TFVLLIGKSI AWYYNASSEL MTYDEASAYC |
| 41 | QRDYTHLVAI QNKEEINYLN STLRYSPSYY WIGIRKVNNV |
| 81 | WIWVGTQKPL TEEAKNWAPG EPNNKQRNED CVEIYIQRPK |
| 121 | DSGMWNDERC DKKKLALCYT ASCTNTSCSG HGECVETINS |
| 161 | YTCKCHPGFL GPKCDQVVTC QEQEYPDHGS LNCTHPFGLF |
| 201 | SYNSSCSFSC ERGYVPSSME TTVRCTSSGE WSAPAPACHV |
| 241 | VECKALTQPA HGVRKCSSNP GSYPWNTTCT FDCEEGYRRV |
| 281 | GAQNLQCTSS GVWDNEKPSC KAVTCDAIPR PQNGSVSCSN |
| 321 | STAGALAFKS SCNFTCEHSF TLQGPAQVEC SAQGQWTPQI |
| 361 | PVCKASQCEA LSAPQRGHMK CLPSASAPFQ SGSSCKFSCD |
| 401 | EGFELKGSRR LQCGPRGEWD SEKPTCAGVQ CSSLDLPGKM |
| 441 | NMSCSGPAVF GTVCEFTCPE GWTLNGSSIL TCGATGRWSA |
| 481 | MLPTCEAPAN PPRPLVVALS VAATSLLTLS SLIYVLKRFF |
| 521 | WKKAKKFVPA SSCQSLQSFE NYQGPSYII |

The mature sequence for this rat E-selectin extends from about amino acid 22 to amino acid 549. The sequence for this mature rat E-selectin polypeptide is therefore as follows (SEQ ID NO:13).

| | |
|---|---|
| 22 | WYYNASSEL MTYDEASAYC |
| 41 | QRDYTHLVAI QNKEEINYLN STLRYSPSYY WIGIRKVNNV |
| 81 | WIWVGTQKPL TEEAKNWAPG EPNNKQRNED CVEIYIQRPK |
| 121 | DSGMWNDERC DKKKLALCYT ASCTNTSCSG HGECVETINS |
| 161 | YTCKCHPGFL GPKCDQVVTC QEQEYPDHGS LNCTHPFGLF |
| 201 | SYNSSCSFSC ERGYVPSSME TTVRCTSSGE WSAPAPACHV |
| 241 | VECKALTQPA HGVRKCSSNP GSYPWNTTCT FDCEEGYRRV |
| 281 | GAQNLQCTSS GVWDNEKPSC KAVTCDAIPR PQNGSVSCSN |
| 321 | STAGALAFKS SCNFTCEHSF TLQGPAQVEC SAQGQWTPQI |
| 361 | PVCKASQCEA LSAPQRGHMK CLPSASAPFQ SGSSCKFSCD |
| 401 | EGFELKGSRR LQCGPRGEWD SEKPTCAGVQ CSSLDLPGKM |
| 441 | NMSCSGPAVF GTVCEFTCPE GWTLNGSSIL TCGATGRWSA |
| 481 | MLPTCEAPAN PPRPLVVALS VAATSLLTLS SLIYVLKRFF |
| 521 | WKKAKKFVPA SSCQSLQSFE NYQGPSYII |

An extracellular E-selectin domain may be used for tolerization of a subject. The extracellular domain of the rat E-selectin provided above includes a sequence of about amino acid 22 to about amino acid 494 and therefore has the following sequence (SEQ ID NO:14).

| | |
|---|---|
| 21 | AWYYNASSEL MTYDEASAYC |
| 41 | QRDYTHLVAI QNKEEINYLN STLRYSPSYY WIGIRKVNNV |
| 81 | WIWVGTQKPL TEEAKNWAPG EPNNKQRNED CVEIYIQRPK |
| 121 | DSGMWNDERC DKKKLALCYT ASCTNTSCSG HGECVETINS |
| 161 | YTCKCHPGFL GPKCDQVVTC QEQEYPDHGS LNCTHPFGLF |
| 201 | SYNSSCSFSC ERGYVPSSME TTVRCTSSGE WSAPAPACHV |
| 241 | VECKALTQPA HGVRKCSSNP GSYPWNTTCT FDCEEGYRRV |
| 281 | GAQNLQCTSS GVWDNEKPSC KAVTCDAIPR PQNGSVSCSN |
| 321 | STAGALAFKS SCNFTCEHSF TLQGPAQVEC SAQGQWTPQI |
| 361 | PVCKASQCEA LSAPQRGHMK CLPSASAPFQ SGSSCKFSCD |
| 401 | EGFELKGSRR LQCGPRGEWD SEKPTCAGVQ CSSLDLPGKM |
| 441 | NMSCSGPAVF GTVCEFTCPE GWTLNGSSIL TCGATGRWSA |
| 481 | MLPTCEAPAN PPRP |

One example of a mouse E-selectin polypeptide sequence that can be found in the NCBI database is the mouse E-selectin sequence with accession number B42755 (gi: 25295806). This mouse E-selectin sequence is the precursor sequence and is provided below for easy reference (SEQ ID NO:15).

| | |
|---|---|
| 1 | MNASRFLSAL VFVLLAGEST AWYYNASSEL MTYDEASAYC |
| 41 | QRDYTHLVAI QNKEEINYLN SNLKHSPSYY WIGIRKVNNV |
| 81 | WIWVGTGKPL TEEAQNWAPG EPNNKQRNED CVEIYIQRTK |
| 121 | DSGMWNDERC NKKKLALCYT ASCTNASCSG HGECIETINS |
| 161 | YTCKCHPGFL GPNCEQAVTC KPQEHPDYGS LNCSHPFGPF |
| 201 | SYNSSCSFGC KRGYLPSSME TTVRCTSSGE WSAPAPACHV |
| 241 | VECEALTHPA HGIRKCSSNP GSYPWNTTCT FDCVEGYRRV |
| 281 | GAQNLQCTSS GIWDNETPSC KAVTCDAIPQ PQNGFVSCSH |
| 321 | STAGELAFKS SCNFTCEQSF TLQGPAQVEC SAQGQWTPQI |
| 361 | PVCKAVQCEA LSAPQQGNMK CLPSASGPFQ NGSSCEFSCE |
| 401 | EGFELKGSRR LQCGPRGEWD SKKPTCSAVK CDDVPRPQNG |
| 441 | VMECAHATTG EFTYKSSCAF QCNEGFSLHG SAQLECTSQG |
| 481 | KWTQEVPSCQ VVQCPSLDVP GKMNMSCSGT AVFGTVCEFT |
| 521 | CPDDWTLNGS AVLTCGATGR WSGMPPTCEA PVSPTRPLVV |
| 561 | ALSAAGTSLL TSSSLLYLLM RYFRKKAKKF VPASSCQSLQ |
| 601 | SFENYHVPSY NV |

The mature sequence for this mouse E-selectin extends from about amino acid 22 to amino acid 612. The sequence for this mature mouse E-selectin polypeptide is therefore as follows (SEQ ID NO:16).

| | |
|---|---|
| 22 | WYYNASSEL MTYDEASAYC |
| 41 | QRDYTHLVAI QNKEEINYLN SNLKHSPSYY WIGIRKVNNV |
| 81 | WIWVGTGKPL TEEAQNWAPG EPNNKQRNED CVEIYIQRTK |
| 121 | DSGMWNDERC NKKKLALCYT ASCTNASCSG HGECIETINS |
| 161 | YTCKCHPGFL GPNCEQAVTC KPQEHPDYGS LNCSHPFGPF |
| 201 | SYNSSCSFGC KRGYLPSSME TTVRCTSSGE WSAPAPACHV |
| 241 | VECEALTHPA HGIRKCSSNP GSYPWNTTCT FDCVEGYRRV |
| 281 | GAQNLQCTSS GIWDNETPSC KAVTCDAIPQ PQNGFVSCSH |
| 321 | STAGELAFKS SCNFTCEQSF TLQGPAQVEC SAQGQWTPQI |
| 361 | PVCKAVQCEA LSAPQQGNMK CLPSASGPFQ NGSSCEFSCE |
| 401 | EGFELKGSRR LQCGPRGEWD SKKPTCSAVK CDDVPRPQNG |
| 441 | VMECAHATTG EFTYKSSCAF QCNEGFSLHG SAQLECTSQG |
| 481 | KWTQEVPSCQ VVQCPSLDVP GKMNMSCSGT AVFGTVCEFT |
| 521 | CPDDWTLNGS AVLTCGATGR WSGMPPTCEA PVSPTRPLVV |
| 561 | ALSAAGTSLL TSSSLLYLLM RYFRKKAKKF VPASSCQSLQ |
| 601 | SFENYHVPSY NV |

An extracellular E-selectin domain may be used for tolerization of a subject. The extracellular domain of the mouse E-selectin provided above includes a sequence of about amino acid 22 to about amino acid 557 and therefore has the following sequence (SEQ ID NO:17).

| | |
|---|---|
| 22 | WYYNASSEL MTYDEASAYC |
| 41 | QRDYTHLVAI QNKEEINYLN SNLKHSPSYY WIGIRKVNNV |
| 81 | WIWVGTGKPL TEEAQNWAPG EPNNKQRNED CVEIYIQRTK |
| 121 | DSGMWNDERC NKKKLALCYT ASCTNASCSG HGECIETINS |
| 161 | YTCKCHPGFL GPNCEQAVTC KPQEHPDYGS LNCSHPFGPF |
| 201 | SYNSSCSFGC KRGYLPSSME TTVRCTSSGE WSAPAPACHV |
| 241 | VECEALTHPA HGIRKCSSNP GSYPWNTTCT FDCVEGYRRV |
| 281 | GAQNLQCTSS GIWDNETPSC KAVTCDAIPQ PQNGFVSCSH |
| 321 | STAGELAFKS SCNFTCEQSF TLQGPAQVEC SAQGQWTPQI |
| 361 | PVCKAVQCEA LSAPQQGNMK CLPSASGPFQ NGSSCEFSCE |
| 401 | EGFELKGSRR LQCGPRGEWD SKKPTCSAVK CDDVPRPQNG |
| 441 | VMECAHATTG EFTYKSSCAF QCNEGFSLHG SAQLECTSQG |
| 481 | KWTQEVPSCQ VVQCPSLDVP GKMNMSCSGT AVFGTVCEFT |
| 521 | CPDDWTLNGS AVLTCGATGR WSGMPPTCEA PVSPTRP |

Another example of a mouse E-selectin polypeptide sequence that can be found in the NCBI database is the mouse E-selectin sequence with accession number NP_{—}035475.1 (gi: 6755452). This mouse E-selectin sequence has the signal sequence and is provided below for easy reference (SEQ ID NO:18).

| | |
|---|---|
| 1 | MGWSWIFLFL LSGTASVHSW YYNASSELMT YDEASAYCQR |
| 41 | DYTHLVAIQN KEEINYLNSN LKHSPSYYWI GIRKVNNVWI |
| 81 | WVGTGKPLTE EAQNWAPGEP NNKQRNEDCV EIYIQRTKDS |
| 121 | GMWNDERCNK KKLALCYTAS CTNASCSGHG ECIETINSYT |
| 161 | CKCHPGFLGP NCEQAVTCKP QEHPDYGSLN CSHPFGPFSY |
| 201 | NSSCSFGCKR GYLPSSMETT VRCTSSGEWS APAPACHVVE |
| 241 | CEALTHPAHG IRKCSSNPGS YPWNTTCTFD CVEGYRRVGA |
| 281 | QNLQCTSSGI WDNETPSCKA VT |

When the SEQ ID NO:18 mouse E-selectin sequence does not have the signal sequence, it has the following sequence (SEQ ID NO:19).

| | |
|---|---|
| 1 | W YYNASSELMT YDEASAYCQR |
| 41 | DYTHLVAIQN KEEINYLNSN LKHSPSYYWI GIRKVNNVWI |
| 81 | WVGTGKPLTE EAQNWAPGEP NNKQRNEDCV EIYIQRTKDS |
| 121 | GMWNDERCNK KKLALCYTAS CTNASCSGHG ECIETINSYT |
| 161 | CKCHPGFLGP NCEQAVTCKP QEHPDYGSLN CSHPFGPFSY |
| 201 | NSSCSFGCKR GYLPSSMETT VRCTSSGEWS APAPACHVVE |
| 241 | CEALTHPAHG IRKCSSNPGS YPWNTTCTFD CVEGYRRVGA |
| 281 | QNLQCTSSGI WDNETPSCKA VT |

Sources of E-selectin that can be used with the current invention include E-selectin that has been substantially purified from natural sources, recombinant E-selectin produced in prokaryotic or eukaryotic host cells by methods available in the art, and fragments of E-selectin. Furthermore, small organic molecules or peptides with structures that mimic an immunoreactive portion of E-selectin can also be used.

In some embodiments, the E-selectin is produced by recombinant procedures. For example, a codon-optimized nucleic acid encoding the mouse E-selectin polypeptide with SEQ ID NO:18, with the following sequence (SEQ ID NO:20) can be used for recombinant production of mouse E-selectin.

| | |
|---|---|
| 1 | ATGGGTTGGT CCTGGATCTT CCTGTTTCTC TTGTCTGGCA |
| 41 | CCGCTAGCGT GCACTCATGG TACTATAACG CCTCGAGTGA |
| 81 | GCTTATGACT TACGACGAAG CGTCCGCATA CTGCCAGCGT |
| 121 | GATTATACAC ATCTGGTCGC TATTCAAAAT AAGGAGGAAA |
| 161 | TCAACTACCT CAATTCTAAC TTGAAACACA GCCCCTCATA |
| 201 | CTATTGGATT GGAATCCGCA AGGTTAACAA TGTATGGATC |
| 241 | TGGGTGGGTA CGGGCAAACC TCTTACCGAG GAAGCCCAGA |
| 281 | ACTGGGCGCC AGGAGAGCCG AACAATAAGC AAAGGAACGA |
| 321 | AGATTGTGTC GAGATTTACA TCCAGAGAAC TAAGGATTCG |
| 361 | GGTATGTGGA ACGACGAACG ATGCAATAAA AAGAAGCTGG |
| 401 | CACTCTGTTA CACAGCTAGT TGCACGAACG CCTCCTGTTC |
| 441 | TGGCCATGGA GAGTGCATTG AGACCATCAA CAGCTATACT |
| 481 | TGCAAATGTC ACCCCGGTTT CTTGGGCCCT AATTGCGAAC |
| 521 | AAGCTGTTAC ATGTAAGCCA CAGGAGCACC CGGATTACGG |
| 561 | ATCACTGAAC TGCTCCCATC CCTTCGGTCC TTTTTCGTAC |
| 601 | AATAGTTCTT GCAGCTTCGG CTGTAAACGT GGATATCTTC |
| 641 | CATCATCCAT GGAAACCACG GTACGCTGCA CTTCGAGTGG |
| 681 | TGAGTGGTCT GCGCCGGCCC CCGCATGTCA CGTGGTCGAA |
| 721 | TGCGAGGCTC TCACCCATCC TGCCCACGGC ATCAGGAAGT |
| 761 | GCAGCTCCAA CCCAGGATCA TACCCCTGGA ACACAACTTG |
| 801 | TACCTTCGAC TGCGTTGAAG GTTACAGACG TGTGGGCGCG |
| 841 | CAAAATTTGC AGTGTACGTC GTCTGGAATT TGGGACAACG |
| 881 | AGACACCTAG TTGCAAGGCT GTCACTTAA |

Recombinant procedures for production of E-selectin polypeptides can employ expression systems for small or large scale production of E-selectin. Expression systems useful for making E-selectin include, but are not limited to, cells or microorganisms that are transformed with a recombinant nucleic acid construct that contains a nucleic acid segment encoding an E-selectin polypeptide. Examples of recombinant nucleic acid constructs may include bacteriophage DNA, plasmid DNA, cosmid DNA, or viral expression vectors. Examples of cells and microorganisms that may be transformed include bacteria (for example, E. coli or B. subtilis); yeast (for example, Saccharomyces and Pichia); insect cell systems (for example, baculovirus in Spodoptera frugiperda, Sf9 cells); plant cell systems; or mammalian cell systems (for example, COS, CHO, BHK, 293, VERO, HeLa, MDCK, W138, and NIH 3T3 cells). Also useful as host cells are primary or secondary cells obtained directly from a mammal that are transfected with a plasmid vector or infected with a viral vector. Examples of suitable expression vectors include, without limitation, plasmids and viral vectors such as herpes viruses, retroviruses, vaccinia viruses, attenuated vaccinia viruses, canary pox viruses, adenoviruses, adeno-associated viruses, lentiviruses and herpes viruses, among others. Synthetic methods may also be used to produce polypeptides and peptide fragments of the invention. Such methods are known and have been reported. Merrifield, Science, 85:2149 (1963).

In some embodiments, the expression system includes use of Chinese Hamster Ovary (CHO) cells or insect cells as the host cells. The glycosylation with a mammalian cell such as a CHO cell is known to differ from that of an insect expression system such as the baculovirus expression vector system. The difference is that glycosylation of a protein molecule derived from the baculovirus vector inserted into an insect expression system leads to an asparagine attached di-N-acetylglycosamine to which a terminal trimannose is attached. This is termed the paucimannose structure and it facilitates interaction with mannose receptors on antigen-presenting cells. Hence, there may be an advantage in some situations to utilize a baculovirus expression vector system. In other embodiments, a mammalian expression system may be used, where additional N-linked glycans may be attached to the three mannoses of the terminal trimannose (paucimannose) structure generated in the insect expression system. These N-linked glycans include N-acetylglycosamine, galactose, and N-acetylneuraminic acid (also known as sialic acid). Therefore, a variety of host cells can be used to generate E-selectin polypeptides with somewhat different glycosylation patterns. The invention is directed to compositions and methods of using E-selectin with any type of glycosylation, or no glycosylation.

### Immune Tolerance

The immune system has the remarkable ability to mount a highly specific response against invading pathogens while ignoring self molecules. This specificity is determined in part by the T lymphocyte, which expresses a randomly generated and unique T-cell receptor (TCR) that recognizes a peptide antigen bound to a major histocompatibility complex (MHC) molecule. MHC molecules can bind both to self peptides as well as to foreign peptides, where the self peptides are from the same organism as the MHC molecules (i.e., the host) and the foreign peptides are from a different, foreign organism. Thus, the specificities of the peripheral TCR repertoire and/or the function of self-reactive T cells must be regulated such that the immune system ignores the self peptides or responds in a way that does not injure the host. The physical elimination of autoreactive T cells during thymocyte development is the primary mechanism used by the immune system to establish such self-tolerance. However, not all self peptides are present in the thymus. Therefore, the immune system must either ignore a tissue-specific self peptide, or develop an active self-tolerance that relies on the suppression, physical elimination, or functional inactivation of mature autoreactive T cells.

The following observations are generally applicable to immune tolerance: (1) tolerance refers to a selective inability of the immune system to respond to antigens and, for purposes of this invention, is a “learned” phenomenon; (2) both foreign and self-antigens can be targets of tolerogenic processes; (3) although tolerance can be mediated by suppressor cells, tolerance is not the same as immune suppression, either mechanistically or clinically; (4) tolerance can be maintained by active or passive processes and can result from cell inactivation, altered cellular function, or cell death; and (5) tolerance can be induced centrally (in the thymus) or peripherally.

Methods and pharmaceutical formulations have been described that induce tolerance, orally or mucosally (e.g., by intranasal administration, using autoantigens, bystander antigens, or disease-suppressive fragments or analogs of autoantigens or bystander antigens). Such treatments are described in Weiner et al., “Bystander suppression of autoimmune diseases,” WO 93/16724 (1993); and “Enhancement of the down-regulation of autoimmune diseases by oral administration of autoantigens,” WO 91/12816 (1991); Weiner et al., “Improved treatment of autoimmune diseases by aerosol administration of auto antigens,” WO 91/08760 (1991); Weiner et al., “Methods of treating or preventing autoimmune uveoretinitis in mammals,” WO 91/01333 (1991); Weiner et al., “Method of treating or preventing type 1 diabetes by oral administration of insulin,” WO 92/06704 (1992); Hafler et al., “Bystander suppression of retroviral-associated neurological disease,” WO 94/0121 (1994); Weiner et al., “Method of treating rheumatoid arthritis with type II collagen,” WO 94/07520 (1994); Weiner et al., “Methods and compositions for suppressing allograft rejection in mammals,” WO 92/07581 (1992); Wucherpfenning et al., “Multiple sclerosis T cell receptor,” WO 91/15225 (1991); Weiner et al., “Suppression of proliferative response and induction of tolerance with polymorphic class II MHC allopeptides,” WO 93/20842 (1993); Weiner et al., “Suppression of T cell proliferation using peptide fragments of myelin basic protein,” WO 93/21222 (1993) and Weiner et al., “Treatment of autoimmune diseases by oral administration of autoantigens,” WO 92/06708 (1992).

According to the invention, immune tolerance can be generated by exposure of mucosal surfaces to E-selectin. Immune responses in mucosal tissues are self-limited, and repeated challenge with selected antigens such as E-selectin results in a diminished response. Mucosal administration of both high- and low-dose antigen can result in immune tolerance, in which the immune response to subsequent systemic administration of antigen is blocked. However, at least two mechanisms of immune tolerance may exist. Tolerance to high-doses of an antigen appears to occur by inactivation or clonal deletion of Th1 and Th2 cells. In contrast, tolerance to low doses of antigen leads to “bystander” immune suppression mediated by stimulation of regulatory cells to produce Th2- and Th3 type cytokines, with interleukin-4 (IL-4), interleukin-10 (IL-10) and TGF-β being the major suppressive cytokines.

Intravenous administration of autoantigens (and fragments thereof containing immunodominant epitopic regions) has been found to induce immune suppression through a mechanism called clonal anergy. Although clonal anergy was originally described using a tissue culture system of cloned T cells, it has since been defined as a reversible, induced tolerance state in which the T lymphocyte cannot produce its autocrine growth factor IL-2 or proliferate in response to the antigen it recognizes. In vitro, this unresponsive state is induced by stimulation of the T cell through its TCR in the absence of costimulatory signals, such as those occurring as a result of the interaction of B7 molecules on the antigen presenting cell (APC) with CD28 receptors on the T cell. In the absence of such costimulatory signals, T cells fail to proliferate, and TCR occupancy unaccompanied by proliferation down-regulates the T cell's responsiveness. Clonal anergy causes deactivation of only immune attack T cells specific to a particular antigen, the result being a significant reduction in the immune response to this antigen. Thus, the autoimmune response-promoting T cells specific to an autoantigen, once anergized, no longer proliferate in response to that antigen. This reduction in proliferation also reduces the immune reactions responsible for autoimmune disease symptoms (such as neural tissue damage that is observed in MS). There is also evidence that oral administration of autoantigens (or immunodominant fragments) in a single dose and in substantially larger amounts than those that trigger “active suppression” may also induce tolerance through clonal anergy (or clonal deletion). Active suppression functions via a different mechanism from that of clonal anergy.

Bystander suppression can also involve induction of regulatory cells in mucosal tissues that are specific for the mucosally administered antigen. So called “bystander antigens” cause regulatory (suppressor) T cells to be induced in the gut-associated lymphoid tissue (GALT), or bronchial associated lymphoid tissue (BALT), or most generally, mucosa associated lymphoid tissue (MALT). MALT includes both GALT and BALT. After migration to the diseased or affected organ, these regulatory cells can be activated by the presence of the antigen, and will secrete immunosuppressive cytokines (IL-4, IL-10, and TGF-β), thereby leading to suppression of ongoing immune responses to the antigen against which tolerance was induced and to unrelated self antigens. Evidence suggests that immune regulation and bystander suppression occur after administration of intermediate or lower antigen doses, whereas clonal deletion or clonal anergy of antigen-reactive lymphocytes generally occurs at high dosages.

IL-4, IL-10 and TGF-β are antigen-nonspecific immunosuppressive factors that suppress immune attack regardless of the antigen that triggers the attack.

However, because oral or mucosal tolerization with a bystander antigen only causes the release of these cytokines in the vicinity of autoimmune attack, no systemic immunosuppression ensues. TGF-β is thought to be one of the most important cytokines for bystander tolerance. IL-4 enhances Th2 response (i.e., acts on T-cell precursors and causes them to differentiate preferentially into Th2 cells at the expense of Th1 responses). IL-4 also indirectly inhibits Th1 exacerbation. IL-10 is a direct inhibitor of Th1 responses.

After orally administering bystander antigens to mammals afflicted with autoimmune disease conditions, increased levels of TGF-β, IL-4, and IL-10 are observed at the locus of autoimmune attack (Chen et al., Science, 265:1237 (1994)). The bystander suppression mechanism has also been confirmed by von Herrath et al., J. Clin. Invest., 96:1324 (1996).

Immune responses in mucosal tissues are self-limited, and repeated challenge with selected antigens results in a diminished response. Mucosal administration of both high- and low-dose antigen results in alterations in the immune response to subsequent systemic administration of antigen. Tolerance to high-doses of an antigen appears to occur by inactivation or clonal deletion of Th1 and Th2 cells. In contrast, tolerance to low doses of antigen leads to “bystander” immune suppression mediated by stimulation of regulatory cells to produce Th2- and Th3 type cytokines, with interleukin-4 (IL-4), interleukin-10 (IL-10) and TGF-β being the major suppressive cytokines.

As described herein, mucosal administration of E-selectin can result in an immune response that is immunosuppressive and location specific (“targeted”). Immune tolerance need not be generated with specific disease-associated autoantigens. Instead, the present invention provides for altering an inflammatory response without using an antigen specific for the underlying response. Therefore, E-selection administration is useful to prevent, inhibit or treat inflammation associated with a number of different inflammatory diseases and autoimmune disorders regardless of whether the disease-associated autoantigen has been identified.

### Conditions and Diseases

According to the invention, E-selectin administration is useful for treating, inhibiting and/or preventing many diseases and conditions. For example, E-selectin administration can inhibit, prevent and/or treat vascular dementia, strokes and other forms of vascular disease, as well as autoimmune diseases, inflammatory conditions, and demyelinating diseases. Additionally, it can be used in treating disorders in which E-selectin has been determined, or may be determined, to play a role, such as, for example, lung injury, psoriasis, contact dermatitis, inflammatory bowel disease, arthritis, and the like. (See, e.g., Washington R., et al., “Expression of immunologically relevant endothelial cell activation antigens on isolated central nervous system microvessels from patients with multiple sclerosis,” Ann. Neurol. 35: 89 (1994); Bevilacqua (1989); Bevilacqua and Nelson, “Selectins,” J. Clin. Invest. 91: 379 (1993); Koch, et al., “Immunolocalization of endothelial and leukocyte adhesion molecules in human rheumatoid and osteoarthritic synovial tissues,” Lab Invest. 64: 313 (1991); Mulligan, et al., “Role of endothelial-leukocyte adhesion molecule 1 (ELAM-1) in neutrophil-mediated lung injury in rats,” J. Clin. Invest. 88: 1396 (1991); and Mulligan, et al., “Protective effects of oligosaccharides in P-selectin-dependent lung injury,” Nature 364: 149 (1993)).

Stroke: Stroke, defined as a sudden weakening or loss of consciousness, sensation and voluntary motion caused by rupture or obstruction of an artery of the brain, is the third cause of death in the United States. Worldwide, stroke is the number one cause of death due to its particularly high incidence in Asia. Ischemic stroke is the most common form of stroke, being responsible for about 85% of all strokes, whereas hemorrhagic strokes (e.g. intraparenchymal or subarachnoid) account for the remaining 15%. Due to the increasing mean age of the population, the number of strokes is continuously increasing. Because the brain is highly vulnerable to even brief ischemia and recovers poorly, primary prevention in ischemic stroke prevention offers the greatest potential for reducing the incidence of this disease.

The current invention provides methods of reducing the likelihood of a stroke by administration of E-selectin to mucosal tissues. Although not wishing to be limited by theory, reduction of the incidence of stroke may occur by induction of tolerance to E-selectin. Such E-selectin administration may specifically reduce the incidence of intracranial hemorrhage, for example, because evidence is available on the role of TGF-β and the incidence of intracranial hemorrhage. Mutations of the endoglin gene are associated with intracranial hemorrhage in patients (Alberts, M. J. et al., Ann. Neurol., 41: 683 (1997)). Endoglin appears to bind TGF-β and subsequently plays a role in vascular maintenance and development. Impairment of endoglin function appears to diminish the response of the endothelium to TGF-β resulting in an increased tendency to hemorrhage. E-selectin administration as provided herein increases the number of TGF-β positive lymphocytes and may increase the release of TGF-β in vessel segments that are becoming activated, as described herein. This can reduce the likelihood of hemorrhage in the presence of endoglin. This is potentially relevant to the observed elimination of intracranial hemorrhage in the group that received E-selectin tolerization and booster tolerization, as described in Examples section below.

In another aspect, the current invention provides a method for mitigating brain tissue damage following a stroke by administering E-selectin to a patient immediately after, or preferably before occurrence of the stroke. Preferably, E-selectin is administered in a manner that induces tolerance, as described herein, most preferably bystander-effect tolerance. Considerations regarding E-selectin sources, doses, delivery routes, formulations, and the like, are described herein for methods of preventing a stroke. As shown in the Examples provided herein, not only does E-selectin administration significantly reduce the number of infarcts formed in a rat model prone to spontaneous strokes, the infarcts that form are significantly smaller in size than control infarcts. Therefore, administration of E-selectin can minimize brain tissue damage in animals (including humans) that have a stroke, particularly when the animals (and humans) are in a state of E-selectin tolerance.

Vascular dementia: Vascular dementia is comprised of a number of heterogeneous pathological conditions, which results from ischemic or hemorrhagic brain lesions as well as from lesions that develop during protracted hypoperfusion. The subcortical ischemic form of vascular dementia is a common type of vascular cognitive impairment and dementia, and one of the major causes of cognitive decline in elderly people. Subcortical ischemic vascular dementia mainly results from small-vessel disease, which causes lacunes and extensive white matter lesions, and can be compared to large vessel dementia or cortical vascular dementia (Roman G C, Neurology. 1993; 43:250-260, Roman GC Lancet Neurol. 2002; 1:426-436). The ischemic lesions in subcortical ischemic vascular dementia particularly affect the frontal-subcortical circuits an observation that explains the major cognitive and clinical neurological effects of vascular dementia (Ishii N, Neurology 1986; 36: 340-45, Cummings J L, Arch Neurol 1993; 50:873-80). Subcortical ischemic vascular dementia is also caused by persistent hypertension (de Leeuw F E, Brain. 2002; 125:765-772) and hypoperfusion due to congestive heart failure (Roman G C. Neurol Res. 2004; 26:454-458), atrial fibrillation (de Leeuw F E, Neurology. 2000; 54:1795-1801), and obstructive sleep apnea (Kamba M, J. Neurol. Neurosurg. Psychiatry. 2001; 71; 334-339).

Ischemic white matter lesions, a common finding in elderly people, are the characteristic pathological changes in subcortical ischemic vascular dementia and cognitive impairment, and cognitive dysfunctions are related to lesion severity (Hachinski V C, Arch Neurol. 1987; 4:21-23, Pantoni L, Alzheimer Dis. Assoc. Disord. 1999; 13(suppl 3):S49S54, de Groot J C, Neurology 2001; 56:1539-1545). Cerebrovascular white matter lesions constitute the core pathology in several types of vascular dementia, such as Binswanger's disease, cerebral amyloid angiopathy, and cerebral autosomal dominant arteriopathy with subcortical infarcts and leukoencephalopathy (CADASIL). These cerebrovascular white matter lesions are caused by chronic cerebral hypoperfusion, which result from the severe stenosis of several arteries or arterioles mainly in deep white matter (Pantoni L, Stroke 1997; 28:652-659, de Groot J C, Neurology 2001; 56:1539-1545, Roman G C, Neurol. Res. 2004; 26:454-458, Capizzano A A, Am J Neuroradiol 2000; 21:621-630).

Animal models exist for vascular dementia, permitting analysis of the effects of drugs and drug dosages on the development, prognosis and recovery from vascular dementia. In particular, cerebrovascular white matter lesions can be experimentally induced in the rat brains as a result of protracted hypoperfusion induced by the permanent occlusion of both common carotid arteries (Wakita H, Acta Neuropathol. (Berl) 1994; 87: 484-492). In this model, cerebral blood flow decreases to about 33 to about 82% of normal over extended periods of time (Tsuchiya M, Exp. Brain Res. 89:87-92 (1992): Otori T, Cerebrovasc. Dis. 6(suppl): 71 (1996); Tomimoto H, Brain Nerve 49:639-644 (1997); Ouchi Y, J Nucl Med. 39:198-202 (1998)). These animals exhibit delayed white matter lesions and memory impairment correlated with the damage of frontal-subcortical circuits. This method of inducing forebrain ischemia can thus be used as a model for vascular dementia (Wakita H, Acta Neuropathol. (Berl) 87: 484-492 (1994); Pappas B A, Brain Res. 708:50-58 (1996); Ohta H, Neuroscience 79:1039-1050 (1997); Wakita H, Brain Res. 924:63-70 (2002)); Sarti C, Behav Brain Res. 136:13-20 (2002)).

Previous studies using this animal model for vascular dementia have demonstrated that CD4- or CD8-positive T cells infiltrate in the neural parenchyma, and that microglia, the immune effector cells of the central nervous system, are activated and express MHC class I and II antigens briefly after ischemia in a manner predictive of the extent and the severity of demyelination and axonal damage (Wakita H, Acta Neuropathol. (Berl) 87: 484-492 (1994); Wakita H, Stroke 26:1415-1422 (1995); Wakita H, Brain Res. 792:105-113 (1998); Wakita H, Neuroreport 14:1461-1465 (1999); Wakita H, Brain Res. 924:63-70 (2002)). The suppression of these activated microglia by immunosuppressive and anti-inflammatory drugs results in an attenuation of the white matter lesions (Wakita H, Stroke 26:1415-1422 (1995); Wakita H, Brain Res. 792:105-113 (1998); Wakita H, Neuroreport 14:1461-1465 (1999); Wakita H., Brain Res. 992:53-59 (2003)). The activation of microglia can also be detected in the early stage of human cerebrovascular white matter lesions, and is associated with degradation of myelin and axonal damage (Suenaga T, Acta Neuropathol (Berl). 87:450-455 (1994); Akiguchi I, Stroke. 28:1423-1429 (1997)). These data suggest that the immunological and inflammatory reactions augment the white matter damage under chronic ischemia.

As described above, E-selectin, a glycoprotein, is a cell surface-bound leukocyte adhesion molecule specific to endothelial cells (Bevilacqua M P, Science 243(4895):1160-1165 (1989)). It mediates the interaction between leukocytes, platelets, and the endothelium (Bevilacqua (1989)). Normal resting endothelial cells do not express E-selectin (Pigott R, BBRC 187:584-9 (1992)). The expression of E-selectin is induced in response to proinflammatory cytokines, such as IL-1 and TNF, and its increased surface expression is a reflection of endothelial activation (Bevilcqua M P, Annu. Rev. Immunol. 11:767-804 (1993)). In patients with cerebrovascular disease, including subcortical ischemic vascular dementia, the serum concentration of the soluble isoform of E-selectin is increased (Fassbender K, Stroke 26:1361-1364 (1995); Frijns C J, Stroke 28: 2214-2218 (1997); Fassbender K, Stroke 30:1647-1650 (1999)). The up-regulation of E-selectin expression in the ischemic cerebral vasculature has been shown in experimental cerebral ischemia (Wang X, Stroke 26:1665-1669 (1995); Haring H-P, Stroke 27:1386-1392 (1996); Zhang R L, J Cereb Blood Flow Metab. 16:1126-113 (1996); Huang J, Stroke 31:3047-3053 (2000)). Moreover, administration of anti-E-selectin antibody reduces the infarct volume and neurological deficits in murine transient focal ischemia model (Huang J, Stroke 31:3047-3053 (2000)).

In view of these observations, and the results provided herein, vessel activation and E-selectin expression play a pivotal role in the inflammatory process and subsequent tissue injury after cerebral ischemia through leukocyte-endothelial attachment and infiltration of leukocytes.

Thus, a novel method to induce generation of regulatory T cells targeted to activating blood vessels has been developed involving administration of E-selectin to induce mucosal tolerance to that antigen. Mucosal tolerance to E-selectin prevents ischemic and hemorrhagic strokes in spontaneously hypertensive stroke prone rats (Takeda H, Stroke 33:2156-2164 (2002)) and protects against ischemic brain damage after permanent middle cerebral artery occlusion in spontaneously hypertensive stroke prone rats (Chen Y, Proc. Natl. Acad. Sci. U.S.A. 100:15107-12 (2003)). These findings suggest that E-selectin participates in inflammation and immunological responses during and after an ischemic insult and serves to target immunomodulatory regulatory T cells to blood vessel segments that are undergoing endothelial cell activation. As illustrated in a previous application by the inventors U.S. Ser. No. 10/296,423 (filed Jun. 11, 2003, and incorporated herein in its entirety), these regulatory T cells may prevent stroke and protect against ischemic brain damage through “bystander suppression.”

Autoimmune and/or Demyelinating Disorders: Over the past four decades, the list of diseases associated with autoantibodies against tissues, cells, or specific autoantigens has grown enormously (Rose et al., Autoimmune Diseases, 3rd ed., Academic Press Inc., San Diego, Calif. 1998; Peter et al., Autoantibodies, Elsener Science B. V. Amsterdam, The Netherlands (1996)). The classification of a disease as autoimmune has traditionally been based on the detection of autoantibodies that could be visualized reacting with an affected tissue or cell. Technological advances, in particular the development of microscopes that allow for more sensitive detection of cell surface-bound autoantibodies resulted in a proliferation of newly recognized autoimmune disorders.

As was understood early on, the identification of individual autoantigens is key not only to uncovering the etiology and pathogenesis of an autoimmune disease, but also to improving the autoantibody assay used to diagnose or verify a disease. Improved biochemical and molecular methods have allowed a rapid dissection of autoantigens associated with specific autoimmune diseases. For analysis of peptide antigens, the most useful trick has been to use the patient's own autoantibodies as a biochemical tool in immunoprecipitation or immunobloting experiments to identify the autoantigen, which can then be sequenced by standard techniques. Additional approaches have also been developed to characterize non-peptide autoantigens. As can be seen in Table 1, there are numerous autoantigens that have been either cloned and sequenced or purified, many of which are commercially available as recombinant proteins and can be used for specific autoantibody assays.

TABLE 1
Recombinant or purified autoantigens recognized by
autoantibodies associated with human autoimmune disorder

| Autoantigen | Autoimmune disease |
|---|---|
| A. Cell or organ-specific | |
| autoimmunity | |
| Acetylcholine receptor | Myasthenia gravis |
| Actin | Chronic active hepatitis, primary biliary |
| | cirrhosis |
| Adenine nucleotide translocator (ANT) | Dilated cardiomyopathy, myocarditis |
| β-Adrenoreceptor | Dilated cardiomyopathy |
| Aromatic L-amino acid decarboxylase | Autoimmune polyendocrine syndrome |
| | type 1 (APS-1) |
| Asialoglycoprotein receptor | Autoimmune hepatitis |
| Bactericidal/permeability-increasing | Cystic fibrosis vasculitides |
| protein (Bpi) | |
| Calcium-sensing receptor | Acquired hypoparathyroidism |
| Cholesterol side-chain cleavage enzyme | APS-1 |
| (CYPIIa) | |
| Collagen type IV α₃-chain | Goodpasture syndrome |
| Cytochrome P450 2D6 (CYP2D6) | Autoimmune hepatitis |
| Desmin | Crohn disease, coronary artery disease |
| Desmoglein 1 | Pemphigus foliaceus |
| Desmoglein 3 | Pemphigus vulgaris |
| F-actin | Autoimmune hepatitis |
| GM gangliosides | Guillain-Barré syndrome |
| Glutamate decarboxylase (GAD65) | Type 1 diabetes, stiff man syndrome |
| Glutamate receptor (GLUR) | Rasmussen encephalitis |
| H/K ATPase | Autoimmune gastritis |
| 17-α-Hydroxylase (CYP17) | APS-1 |
| 21-Hydroxylase (CYP21) | Addison Disease |
| IA-2 (ICA512) | Type 1 diabetes |
| Insulin | Type 1 diabetes, insulin hypoglycemic |
| | syndrome (Hirata disease) |
| Insulin receptor | Type B insulin resistance, acanthosis, |
| | systemic lupus erythematosus (SLE) |
| Intrinsic factor type 1 | Pernicious anemia |
| Leukocyte function-associated antigen | Treatment-resistant Lyme arthritis |
| (LFA-1) | |
| Myelin-associated glycoprotein (MAG) | Polyneuropathy |
| Myelin basic protein | Multiple sclerosis, demyelinating |
| | diseases |
| Myelin oligodendrocyte glycoprotein | Multiple sclerosis |
| (MOG) | |
| Myosin | Rheumatic fever |
| p-80-Coilin | Atopic dermatitis |
| Pyruvate dehydrogenase complex-E2 | Primary biliary cirrhosis |
| (PDC-E2) | |
| Sodium iodide symporter (NIS) | Graves disease, autoimmune |
| | hypothyroidism |
| SOX-10 | Vitiligo |
| Thyroid and eye muscle shared protein | Thyroid associated ophthalmopathy |
| Thyroglobulin | Autoimmune thyroiditis |
| Thyroid peroxidases | Autoimmune Hashimoto thyroiditis |
| Thyrotropin receptor | Graves disease |
| Tissue transglutaminase | Coeliac disease |
| Transcription coactivator p75 | Atopic dermatitis |
| Tryptophan hydroxylase | APS-1 |
| Tyrosinase | Vitiligo, metastatic melanoma |
| Tyrosine hydroxylase | APS-1 |
| B. Systemic autoimmunity | |
| ACTH | ACTH deficiency |
| Aminoacyl-tRNA histidyl synthetase | Myositis, dermatomyositis |
| Aminoacyl-tRNA synthetase (several) | Polymyositis, dermatomyositis |
| Cardiolipin | SLE |
| Carbonic anhydrase II | SLE, Sjögren syndrome, systemic |
| | sclerosis |
| Collagen (multiple types) | Rheumatoid arthritis (RA), SLE, |
| | progressive systemic sclerosis |
| Centromere-associated proteins | Systemic sclerosis |
| DNA-dependent nucleosome-stimulated | Dermatomyositis |
| ATPase | |
| Fibrillarin | Scleroderma |
| Fibronectin | SLE, RA, morphea |
| Glucose-6-phosphate isomerase | RA |
| β2-Glycoprotein 1 (β2-GPI) | Primary antiphospholipid syndrome |
| Golgin (95, 97, 160, 180) | Sjögren syndrome, SLE, RA |
| Heat shock protein | Various immune-related disorders |
| Hemidesmosomal protein 180 | Bullous pemphigoid, herpes gestationis, |
| | cicatricial pemphigoid |
| Histone H2A-H2B-DNA | SLE |
| IgE receptor | Chronic idiopathic urticaria |
| Keratin | RA |
| Ku-DNA-protein kinase | SLE |
| Ku-nucleoprotein | Connective tissue syndrome |
| La phosphoprotein (La 55-B) | Sjögren syndrome |
| Myeloperoxidase | Necrotizing and crescentic |
| | glomerulonephritis (NCGN), systemic |
| | vasculitis |
| Proteinase 3 (PR3) | Wegener granulomatosis, Churg-Strauss |
| | syndrome |
| RNA polymerase I-III (RNP) | Systemic sclerosis, SLE |
| Signal recognition protein (SRP54) | Polymyositis |
| Topoisomerase-I (Scl-70) | Scleroderma, Raynaud syndrome |
| Tubulin | Chronic liver disease, visceral |
| | leishmaniasis |
| Vimentin | Systemic autoimmune disease |
| C. Plasma protein and cytokine | |
| autoimmunity | |
| C1 inhibitor | Autoimmune C1 deficiency |
| C1q | SLE, membrane proliferative |
| Cytokines (IL-1α, IL-β, IL-6, IL-10, | RA, Systemic sclerosis, normal subjects |
| LIF) | |
| Factor II, factor V, factor VII, factor | Prolonged coagulation time |
| VIII, factor IX, factor X, factor XI, factor | |
| XII, thrombin, vWF | |
| Glycoprotein IIb/IIIg and Ib/IX | Autoimmune thrombocytopenia purpura |
| IgA | Immunodeficiency |
| Oxidized LDL (OxLDL) | Atherosclerosis |
| D. Cancer and paraneoplastic | |
| autoimmunity | |
| Amphiphysin | Neuronopathy, small lung cell cancer |
| Cyclin B1 | Hepatocellular carcinoma |
| DNA topoisomerase II | Liver cancer |
| Desmoplakin | Paraneoplastic pemphigus |
| Gephyrin | Paraneoplastic stiff man syndrome |
| Hu proteins | Paraneoplastic encephalomyelitis |
| Neuronal nicotinic acetylcholine receptor | Subacute autonomic neuropathy, cancer |
| p53 | Cancer, SLE |
| p62 (IGF-II mRNA-binding protein) | Hepatocellular carcinoma (China) |
| Recoverin | Cancer-Associated retinopathy |
| Ri protein | Paraneoplastic opsoclonus myoclonus |
| | ataxia |
| β IV spectrin | Lower motor neuron syndrome |
| Synaptotagmin | Lambert-Eaton myasthenic syndrome |
| Voltage-gated calcium channels | Lambert-Eaton myasthenic syndrome |
| Yo protein | Paraneoplastic cerebellar degeneration |

The availability of the autoantigen and its sequence has also made it possible to study autoantigen uptake and processing, the cell-surface presentation of epitopes on HLA class II molecules, and the role of B lymphocytes in autoantibody production. Recombinant autoantigens are also being produced in quantities sufficient to grow crystals for x-ray analysis of the structure, which should yield information crucial to uncovering T and B cell epitopes. Fortunately, alongside of this arduous process, it is possible to search the primary sequence of the autoantigen using several search algorithms based on the structural requirements for T cell receptor recognition of HLA-bound peptides. The availability of autoantigen has made it possible to isolate monoclonal antibodies from patients and to done and sequence the heavy and light chain genes that encode the autoantibodies. Such analyses have revealed that there are many more replacements than silent mutations in the variable gene region and that replacement mutations have accumulated in the complementarity determining regions (CDRs). Since CDRs confer antigen-binding specificity, these studies therefore support the notion that the autoantigen is the driver of autoimmunity.

Most autoimmune responses may be regarded as entirely idiopathic. However, idiopathic autoimmunity often develops in subjects with a certain genetic propensity, most often linked or associated with HLA on chromosome 6. The immune response to an autoantigen may also be initiated by cancer. In other autoimmune disorders, the autoimmune disease is iatrogenic. Therapy with certain drugs may induce an autoimmune reaction. Even in these cases, however, HLA may still be critical to the risk of developing drug- or treatment-associated autoimmunity. Other etiologies are infectious diseases that may cause collateral autoimmune damage. Again, the autoimmune reaction is dependent on an individual's genetic propensity.

The immune system may be fooled into reacting with autoantigens following an initial reaction to infectious agents. One possibility is induction of immune response by molecular mimicry, i.e., a structural similarity between microbial and self-antigens. Several human diseases involve superantigens, molecules that can activate T cells directly through interactions with the variable domain of the T cell receptor β chain. In experimental animals, there is considerable evidence that superantigens can initiate autoimmunity, but their roles in human autoimmunity are less clear, and other mechanisms, such as release of autoantigens following viral lysis, activation of lymphocytes by virus, or bystander activation, may also contribute to these disease processes.

### Animal Models of MS

Animals immunized with components of central nervous system (CNS) myelin develop an autoimmune demyelinating disease of the CNS called experimental autoimmune encephalomyelitis (EAE) (Whitehouse et al., Nature, 224:1322 (1969); Alvord et al., Experimental Allergic Encephalomyelitis: A Useful Model for Multiple Sclerosis, New York Liss (1984); Zamvil et al., Annu. Rev. Immunol., 8:579 (1990)). This disease is the result of a CD4⁺ T cell mediated immune response directed at specific proteins within the CNS leading to demyelination with subsequent axonal and neuronal loss, and it serves as a model of the human disease multiple sclerosis (MS).

In particular, EAE has served as a useful tool for the preclinical testing of new approaches to Th1 autoimmunity. MS is a disease often marked by episodes, with neurological deficits ranging from paralysis to blindness, sensory disturbances and bowel and bladder dysfunction. These episodes last days to weeks and are often followed by periods of remission. Other forms of MS, including primary and secondary progressive MS, do not have these distinctive periods of relapse and remission and the neurological deficits appear and progress. MS often begins in young adulthood and females are affected twice as frequently as males. Thus, MS has several clinical forms, including initial attacks of optic neuritis, episodes of relapsing and remitting paralysis and sensor deficits, and more progressive deterioration without clear relapses or remission. There are EAE models of all of these clinical forms of MS: for example, there is an EAE model for relapsing remitting disease, in which deficits like hind limb paralysis wax and wane after an initial attack (Brocke et al., Methods: A Comparison to Methods in Enzymology, pp. 458-468, Academic Press Inc. (1996)). A single T cell clone reactive to a given region of a myelin protein can induce progressive paralysis leading to death when given in high doses and relapsing and remitting episodes of paralysis when given in smaller doses (Zamvil et al., Nature, 317:355 (1985); Zamvil et al., J. Exp. Med., 162:2107 (1985)). These T cell clones are capable of inducing both inflammation in the perivascular white matter and demyelination of the sheath surrounding the neuronal axon (Zamvil et al., J. Exp. Med., 162:2107 (1985); Zamvil et al., Ann. Rev. Immunol., 8:579 (1990)). Models of EAE reflecting more progressive disease, without clear episodes of relapse and remission, have also been described in Chabas et al., Science, 244:1731 (2001). A model of pure optic neuritis has been constructed in a transgenic mouse with TCRs recognizing the major encephalitogenic epitope of myelin oligodendroglial glycoprotein (MOG) (Betelli et al., J. Exp. Med., 197:1073 (2003); Steinman, J. Exp. Med., 197:1065 (2003)). Models of spontaneous disease in mice made transgenic for crucial human HLA and T cell (TCR) receptor genes have been described (Elmerich et al., J. Immunol., 174:1938 (2005)). These various models of EAE, share many features in common with MS (Table 2).

Similarities between EAE and MS are present on many levels (Steinman, J. Exp. Med., 197:1065 (2003)). The most important factor in genetic susceptibility in both diseases resides in the MHC. In both diseases, CD4 and CD8 T cells can be found in lesions, including evidence of populations of clonally derived T cells, some reactive to myelin proteins. In most EAE models, CD4 T cells predominate in lesions, whereas in MS lesions both CD4 and CD8 T cells are present. Antibodies and complement are found in lesions in both models. EAE and MS are characterized by damage to the myelin sheath and in both the animal models and in MS there is evidence for axonal degeneration (Table 2).

TABLE 2
Similarities between EAE and MS

| Characteristic | EAE | MS |
|---|---|---|
| Genetic susceptibility | Strong association with MHC | Strong association with MHC |
| | II | II |
| | Females more susceptible in | Females more susceptible |
| | certain strains | |
| Environmental triggers | Relapses with earlier | Association with earlier |
| | infection; superantigens | infection |
| | trigger relapses | |
| White matter pathology | Th1 T cells, B cells, CD4 and | Th1 T cells, B cells, CD4 and |
| | CD8 T cells, B cells and | CD8 T cells reactive to myelin |
| | antibodies to myelin in | in lesions |
| | lesions | |
| | Clonal CD4 and CD8 T cells | Clonal CD4 and CD8 T cells |
| | reactive to myelin | reactive to myelin components |
| | components | |
| | Macrophages | Macrophages |
| | Microglia | Microglia |
| | α4β1 integrin | α4β1 integrin |
| | Complement | Complement |
| Grey matter pathology | Axonal degeneration | Axonal degeneration |
| Clinical presentation | Optic neuritis, myelitis, | Optic neuritis, myelitis, |
| | periventricular white matter | periventricular white matter |
| | inflammation | inflammation |
| Clinical forms | Relapsing remitting | Relapsing remitting |
| | Progressive | Progressive |

Most autoreactive T cells are deleted during thymic development. However, thymic deletion of autoreactive T cells cannot be complete. Autoreactive T cells that escape thymic deletion constitute the peripheral T cell repertoire and are kept in check in the circulation by the mechanisms of peripheral tolerance. Autoreactive T cells in the periphery cannot become activated until they encounter antigen in the context of relevant major histocompatibility complex (MHC) molecules, together with appropriate costimulatory signals. In CNS inflammatory diseases like EAE and MS, the T cells then migrate across the blood brain barrier to cause disease (Baron et al., J. Exp. Med., 177:57 (1993)).

In the CNS, autoreactive T cells have to be activated by local antigen presenting cells (APCs), also expressing appropriate costimulatory molecules, to initiate inflammation and tissue injury (Chang et al., J. Exp. Med., 190:733 (1999)). It is generally thought that EAE, and by inference MS, is a DTH-type reaction driven by T cells that have differentiated to a Th1 phenotype (Williams et al., Neurosci., 12:229 (1994)). Although myelin antigen-specific Th1 cells are necessary to initiate the disease, most of the cells seen in EAE lesions are recruited nonspecifically. These infiltrating cells consist mainly of T cells and macrophages and, to a lesser extent, B cells. In some cases, polymorphs are also detected in acute EAE lesions (Sobel et al., J. Immunol., 149:1444 (1992)). Nonspecifically recruited cells are thought to play a major role in the tissue damage. Activated macrophages strip myelin from axons and secrete numerous cytokines including IL-1 and TNF-α, which can perpetuate nonspecific inflammatory reactions and contribute to tissue damage. Therefore, following escape from the thymus, an autoreactive T cell has to undergo several discrete steps to mediate an autoimmune disease: The T cells have to be activated in the peripheral immune compartment, differentiate to attain a pathogenic effector phenotype, express appropriate adhesion molecules to traffic to the target organ, and get reactivated to recruit other cells to mediate tissue injury and develop autoimmune disease.

Naive CD4⁺T lymphocytes triggered by antigen differentiate into at least two subpopulations, each producing its own set of cytokines and mediating distinct effector functions (Mosmann et al., J. Immunol., 136:2348 (1986); Abbas et al., Nature, 383:787 (1996)). Type 1 helper T cells (Th1 cells) produce interleukin 2 (IL-2), tumor necrosis factor β (TNF-β), and interferon-gamma (IFN-γ), activate macrophages, and induce delayed type hypersensitivity (DTI-1) responses. Type 2 (Th2) cells produce IL-4, IL-5, and IL-10, stimulate the production of mast cells, eosinophils, and IgE antibodies, and regulate cell-mediated immunity (Mosmann et al., Ann. Rev. Immunol., 7:145 (1989)). IL-4 and IFN-γ show reciprocal inhibition, and IL-10 inhibits the production of IFN-γ and other Th1 cytokines by interfering with antigen presentation by macrophages (Paul et al., Cell, 76:241 (1994)). These two cell populations cross-regulate one another because their respective cytokines act antagonistically.

Cytokines play a pivotal role in the initiation, propagation, and regulation of tissue-specific autoimmune injury. Cellular and cytokine changes in the CNS have been described in several studies of MBP-induced EAE. Inflammation appears to precede the clinical signs of EAE, CD4⁺ cells predominate initially, but peripherally derived macrophages outnumber CD4⁺ cells as the disease progresses. The autoreactive T cells that induce EAE generally display a Th1 phenotype (Miller et al., Immunol. Today, 15:356 (1994)). During progression of EAE, Th1 cytokines are present in the inflammatory EAE lesions in the CNS, whereas Th2 cytokines are absent, strongly suggesting that Th1 cytokines play a role in the pathogenesis of the disease. Recovery from EAE in mice and rats is associated with an increase in the presence of Th2 and Th3 cells and cytokines in the CNS (Khoury et al., J. Exp. Med., 176:1355 (1992); Kennedy et al., J. Immunol., 149:2496 (1992)). These findings, along with the observation that Th2 cytokines can inhibit the actions of inflammatory Th1 cytokines, suggest that the induction and activation of Th2 cells may prevent EAE and other autoimmune diseases mediated by Th1 cells.

### Administration

One aspect of the current invention is a method for locally preventing or inhibiting inflammation by administering E-selectin to a subject. Another aspect of the current invention is a method for inducing E-selectin tolerance in a subject. These methods involve administering E-selectin to mucosal tissues of a subject. According to the invention any E-selectin that can prevent or inhibit inflammation in the subject or induce immune tolerance in the subject to E-selectin can be used. Thus, for example, an E-selectin with any of SEQ ID NO: 1-19 can be used in the invention.

According to the invention, the compositions and methods of the invention prevent, inhibit, reduce or ameliorate inflammation, axonal and/or neuronal loss. Moreover, the compositions and methods of the invention can induce tolerance to an antigen such as E-selectin. Such beneficial effects can be achieved by administration to many types of mucosal tissues including oral, nasal, enteral, vaginal, rectal and respiratory mucosa. By reducing enzymatic degradation in the gastrointestinal tract, lower doses of antigen may sometimes be used for non-oral routes of administration. In some embodiments, intranasal administration of E-selectin is used.

Preventing, inhibiting or reducing inflammation and axonal/neuronal loss and/or inducing tolerance to an antigen such as E-selectin can be achieved by a single series of E-selectin administrations. Thus, for example, E-selectin may be administration protocol involving a single series of five administrations of E-selectin over a period of two weeks. In other embodiments, this regimen of five administrations over two weeks is repeated at least once. Repeating a series of E-selectin administration is referred to herein a “booster” series of administrations. Thus, a single series of E-selectin dosages is administered within about one to two weeks and the “booster” administrations repeat this series of E-selectin administrations after a period of several weeks without any E-selectin administrations. In some embodiments, this booster regimen is repeated every three weeks for the remainder of the life of the subject.

Dosages, E-selectin sources, formulations, dosage volumes, regimens, and methods for analyzing results aimed at optimizing prevention, inhibition or reduction of inflammation and axonal/neuronal loss, and/or induction of tolerance to E-selectin can vary. Thus, minimum and maximum effective dosages vary depending on the method of administration. Suppression of the clinical and histological changes associated with an inflammatory condition or disease can occur within a specific dosage range, which, however, can vary depending on the organism receiving the dosage, the route of administration, whether E-selectin is administered in conjunction with other co-stimulatory molecules, and the specific regimen of E-selectin administration. For example, in general, nasal administration requires a smaller dosage than oral, enteral, rectal, or vaginal administration.

When E-selectin is administered mucosally, dosages are used that range from about 0.005 micrograms/day to about 500 mg/day, or from about 0.05 micrograms/day to about 50 mg/day. In some embodiments, mucosal dosages are from about 0.5 μg to about 50 mg per administration, or from about 0.5 μg to about 5 mg per administration. In view of the guidelines provided herein, optimization of the dosage necessary for treating, inhibiting reducing inflammation and/or axonal/neuronal loss, and/or for immune suppression involves no more than routine experimentation.

E-selectin formulations of the present invention may comprise inert constituents including pharmaceutically-acceptable carriers, diluents, solubilizing agents, emulsifying agents, salts, and the like, as are available in the art. Preferred E-selectin formulations are intranasal formulations including normal saline solutions, such as, for example, isotonic and physiologically buffered saline solutions and phosphate-buffered saline (PBS) solutions. The total volume of the intranasal formulations is typically less than 1 milliliter, preferably about 100 microliters to about 200 microliters.

For oral or enteral E-selectin formulations for use with the present invention, tablets may be formulated in accordance with conventional procedures employing solid carriers well-known in the art. Capsules employed for oral formulations to be used with the methods of the present invention may be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film-Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982.

Examples of solid carriers include starch, sugar, bentonite, silica, and other commonly used carriers. Further non-limiting examples of carriers and diluents which may be used in the formulations of the present invention include saline, syrup, dextrose, and water.

E-selectin can also be administered in an aerosol or inhaled form. Examples of formulations for tolerizing agents administered by inhalation are provided in Weiner, H. et al., “Improved treatment of autoimmune diseases by aerosol administration of auto antigens,” WO9108760 (1991). The antigens can be administered as dry powder particles or as an atomized aqueous solution suspended in a carrier gas (e.g., air, N.sub.2, and the like).

Dry aerosol in the form of finely divided solid particles of E-selectin that are not dissolved or suspended in a liquid can also be used in the practice of the present invention. E-selectin formulations may be in the form of dusting powders and comprise finely divided particles having an average particle size of between about 1 and 5 microns, preferably between 2 and 3 microns. Finely divided particles may be prepared by pulverization and screen filtration using techniques available in the art. The particles may be administered by inhaling a predetermined quantity of the finely divided or powdered material.

The E-selectin formulations of the present invention may also be administered in soluble form as an aerosol spray using, for example, a nebulizer such as those described in U.S. Pat. No. 4,624,251 issued Nov. 25, 1986; U.S. Pat. No. 3,703,173 issued Nov. 21, 1972; U.S. Pat. No. 3,561,444 issued Feb. 9, 1971; and U.S. Pat. No. 4,635,627 issued Jan. 13, 1971. Other systems of aerosol delivery, such as the pressurized metered dose inhaler (MDI) and the dry powder inhaler (see, e.g., Newman, S. P. in Aerosols and the Lung, Clarke, S. W. and Davia, D. eds. pp. 197-224, Butterworths, London, England, 1984) can be used when practicing the present invention.

One useful animal model for the analysis of E-selectin formulations and their effectiveness in treating or preventing stroke is the stroke-prone and spontaneously hypertensive SHR-SP rat (Okamoto, K. et al., “Establishment of the stroke-prone spontaneously hypertensive rat (SHR),” Circ. Res. (Suppl.) 34, 35: 1 (1974)). SHR-SP rats may be obtained from professor Yukio Yamori, Graduate School of Human and Environmental Studies, Kyoto University, Yoshida Nihonmatsu-cho, Sakyo-ku, Kyoto, 606-8316, Japan. SHR-SP rats typically die of early-onset cardiovascular disease, sometimes as early as 14 weeks of age, although some SHR-SP rats live to more than 56 weeks of age. Frequently, the cardiovascular disease manifests as a stroke in these rats. The occurrence of a stroke in these rats is diagnosed by measuring behavioral status that could be divided into 4 patterns: no abnormalities (grade 1), irritable (grade 2), lethargic (grade 3), akinetic (grade 4) (Yamori, U. et al., Japanese Circulation Journal 46: 274 (1982)).

The brains of SHR-SP rats at the time of death, typically contain numerous infarcts and intraparenchymal hemorrhage areas that can be counted and measured through microscopic analysis of brain sections. Thus, the effectiveness of an E-selectin formulation can be determined by comparing infarct and intraparenchymal hemorrhage numbers and areas in SHR-SP rats that have been treated with control or test E-selectin formulations. The administration regimen can be evaluated in a similar manner. The control formulations can consist of only carrier components or non-specific antigens (e.g., ovalbumin). In addition, the efficacy of a regimen of booster administrations versus a single series of E-selectin administration can be compared. Examples of these procedures and comparisons are disclosed in the Examples section of this specification.

Another useful animal model for the analysis of E-selectin formulations and their effectiveness in treating or preventing vascular dementia involves occlusion of carotid arteries in rats. See, e.g., Sarti et al., Persistent impairment of gait performances and working memory after bilateral common carotid artery occlusion in the adult Wistar rat, BEHAVIOURAL BRAIN RESEARCH 136: 13-20 (2002). Thus, cerebrovascular white matter lesions can be experimentally induced in the rat brain as a result of chronic cerebral hypoperfusion. This model is created by permanent occlusion of both common carotid arteries. For example, Wistar rats can be anesthetized, the bilateral common carotid arteries are exposed through a midline cervical incision and the common carotid arteries are double-ligated with silk sutures bilaterally. The cerebral blood flow (CBF) then initially decreases by about 30 to 50% of the control after ligation. The CBF values later range from 33 to 80% of control after about 1 week to about 1 month.

As illustrated herein, white matter rarefaction is detected in rats with occluded carotid arteries after 3 days or more of ligation Microglia and astroglia were activated after arterial occlusion in a manner that predicts the extent and severity of the subsequent white matter damages. A few lymphocytes, labeled with CD4 or CD8 antibodies, can be detected as scattered in the white matter after arterial occlusion. Other pathological changes include axonal damage and demyelination in white matter lesions. Blood-brain barrier disruptions have also been observed as well as increased matrix metalloproteinase activity in white matter lesions. These changes appear very similar to those in human cerebrovascular white matter lesions. Moreover, these results suggest that inflammatory and immunologic reactions play a role in the pathogenesis of the white matter changes.

Such physiological changes are correlated with learning and memory problems in the occluded carotid artery rat model. Thus, the gait performance of rats with occluded arteries declines over time in comparison with baseline. At 60 and 90 days, rats with bilateral common carotid artery occlusion showed decreased performances on object recognition and Y maze spontaneous alternation test in comparison with sham-operated rats. Thus, this rat model of experimental chronic cerebral hypoperfusion by permanent occlusion of the bilateral common carotid arteries exhibited significant learning impairments along with rarefaction of the white matter. This model is a useful tool to assess the effectiveness of E-selectin tolerization on the pathophysiology of chronic cerebral hypoperfusion, and to provide data for determining optimal dosages and dosage regimens for preventing the cognitive impairment and white matter lesions in patients with cerebrovascular disease.

The effectiveness of an E-selectin formulation for treating or preventing vascular dementia can therefore be determined by observing the gait performance, memory, learning abilities and the incidence and severity of white matter lesions in rats with carotid artery occlusions. Similarly, the E-selectin dosage and administration schedule can be adjusted pursuant to the memory and learning abilities of human patients being treated for vascular dementia.

Another useful model for the analysis of E-selectin formulations and their effectiveness in treating multiple sclerosis (MS) is the experimental autoimmune encephalitis (EAE) animal model. EAE model mice can be generated by administration of myelin proteins, for example, myelin oligodendroglial protein (MOG) peptide amino acids 35-55. In general, an adjuvant such as mycobacterium in incomplete Freund's adjuvant, with pertusis toxin is also administered.

The effectiveness of an E-selectin formulation for treating, inhibiting or preventing nerve damage due to inflammation or inflammation associated with certain autoimmune disorders, e.g., in multiple sclerosis (MS), can therefore be determined by methods known to the art, such as observing or measuring muscle weakness, spasticity, numbness, e.g., impairment of pain, temperature or touch, speech disturbances, vision disturbances, dizziness, vertigo, cognitive changes, fatigue, and the like, in humans or animal models. Similarly, the E-selectin dosage and administration schedule can be adjusted pursuant to the observations of or clinical findings in human patients being treated for spinal cord or brain damage such as that associated with MS.

Assessment of the effect of E-selectin formulations on an immune response to E-selectin can also be made, for example, by determining diminution in certain inflammation markers, such as the number of activated T-cell clones directed against activated vascular tissue. Immunological tolerance can be measured by a number of methods that are well-known in the art. In one preferred embodiment, delayed type hypersensitivity (DTH) response can measured in animals by injecting E-selectin, for example, into the footpad or subcutaneous tissue on the back of an organism to be analyzed and then administering a challenge injection, for example into a footpad or an ear flap, at a later time, typically more than 1 week later, most preferably 2 weeks later. DTH reactions can be measured after the elicitation injection as the increase in swelling at the site of the antigen rechallenge. Footpad or ear swelling can be measured at, for example, 0, 24 and 48 hr after challenge.

In some embodiments, the optimum dosage of E-selectin is one that induces E-selectin tolerance, for example, bystander tolerance. In other embodiments, the optimum dosage of E-selectin is one that generates the maximum protective effect in preventing vascular dementia, stroke and the like. In other embodiments, the optimum dosage of E-selectin is one generating the maximum beneficial effect on damaged tissue caused by arterial occlusion. An effective dosage causes at least a statistically or clinically significant attenuation of at least one marker, symptom, or histological evidence characteristic of disease. Markers, symptoms and histological evidence characteristic of vascular dementia include memory loss, confusion, disturbances in axonal transport, demyelination, axonal and/or neuronal loss, induction of metalloproteinases (MMPs), activation of glial cells, infiltration of lymphocytes, edema, inflammation and immunological reactions that lead to tissue damage and further vascular injury. Stabilization of symptoms or diminution of tissue damage, under conditions wherein control patients or animals experience a worsening of symptoms or tissue damage, is one indicator of efficacy of a suppressive treatment.

Ascertaining the effective dosage range as well as the optimum amount of E-selectin is accomplished using the teachings of the present application as well as any available teachings in the art. For example, an optimum regimen for administering E-selectin is determined in light of the information disclosed herein and well known information concerning administration of bystander antigens and autoantigens. Routine variation of dosages, combinations, and duration of treatment is performed under circumstances wherein the effects of such variations on the organism can be measured.

For example, dosages for mammals and humans can be determined by beginning with a relatively low dose (e.g., 0.005 to 5 micrograms or about 0.5 micrograms) and progressively increasing the dosage while measuring appropriate responses (e.g., number of TGF-β, IL-4, and/or IL-10 secreting cells); number and activation of immune attack T-cells in the blood (e.g., by limiting dilution analysis and ability to proliferate; and/or disease severity). The optimum dosage provides maximal prevention from vascular dementia or the maximum protection from tissue damage caused by vascular occlusion while minimizing undesirable side effects. Potential side effects include the generation of pathogenic autoantibodies (Hu, W. et al., “Experimental mucosal induction of uveitis with the 60-kDa heat shock protein-derived peptide 336-351,” Eur. J. Immunol. 28: 2444 (1998); Genain C. P., et al., “Late complications of immune deviation therapy in a nonhuman primate,” Science 274: 2054 (1996)) or a cytotoxic T lymphocyte response that induces autoimmunity (Blanas E., et al., “Induction of autoimmune diabetes by oral administration of autoantigen,” Science 274: 1707 (1996)).

An effective dosage causes at least a statistically or clinically significant attenuation of at least one symptom of inflammation, stroke or vascular dementia, or at least a statistically or clinically significant attenuation of the occurrence rate or time to onset of neuronal inflammation or vascular occlusion. Preferred dosages for intranasal instillations are from about 0.01 μg to about 100 mg per administration; for humans approximately from about 0.05 μg to 75 mg per administration or about 0.5 μg to 50 mg per administration. For rats, one preferred dosage is 5 μg per administration. Preferred aerosol pharmaceutical formulations may comprise, for example, a physiologically-acceptable buffered saline solution containing between about 0.01 μg to about 100 mg E-selectin per administration; for humans approximately from about 0.05 μg to 75 mg per administration of E-selectin or about 0.5 μg to 50 of E-selectin mg per administration.

In some embodiments, E-selectin is administered in a series of administrations. Typically these administrations are spaced apart over a period of 1 to 2 weeks. For example and as further detailed in the Examples, E-selectin can be administered in five intranasal administrations over a two week period. This protocol can involve administering E-selectin every other day for ten days. Preferably, the administration regimen is repeated in booster administrations that are generally administered several weeks apart. In one embodiment, booster administrations are given after every three weeks. Booster administrations may include a series of administrations, as described above for initial administrations.

Cytokine and non-cytokine synergists can be used in conjunction with E-selectin in the present invention to enhance the effectiveness of E-selectin tolerization. Administration “in conjunction with” encompasses simultaneous and sequential administration, as well as administration in combined form or separately. Oral and parenteral use of cytokine synergists (Type I interferons) has been described in Hafler, D. A. et al., “Treatment of autoimmune disease using oral tolerization and/or type 1 interferon,” WO9527499 (1995). Administration of Th2 enhancing cytokines is described in Weiner H. L., et al., “Treatment of autoimmune disease using oral tolerization and/or Th2-enhancing cytokines,” WO95275000 (1995). For example, IL-4 and IL-10 can be administered in the manner described in Weiner et al. Id.

Non-limiting examples of non-cytokine synergists for use in the present invention include bacterial lipopolysaccharides from a wide variety of gram negative bacteria such as various subtypes of E. coli and Salmonella (LPS, Sigma Chemical Co., St. Louis, Mo.; Difco, Detroit, Mich.; BIOMOL Res. Labs., Plymouth, Pa.), Lipid A (Sigma Chemical Co., St. Louis, Mo.; ICN Biochemicals, Cleveland, Ohio; Polysciences, Inc., Warrington, Pa.); immunoregulatory lipoproteins, such as peptides covalently linked to tripalmitoyl-5-glycarylcysteinyl-seryl-serine (P.sub.3 C55) which can be obtained as disclosed in Deres, K. et al. (Nature, 342: 561-564, “In vivo priming of virus-specific cytotoxic T lymphocytes with synthetic lipopeptide vaccine,” 1989) or “Braun's” lipoprotein from E. coli which can be obtained as disclosed in Braun, V., Biochim. Biophys. Acta 435: 335-337, 1976; and cholera toxin β-chain (CTB) the synergist ability of which has been described (though not in connection with abatement of autoimmune reaction) by Sun, J-B et al., “Cholera toxin B subunit: an efficient transmucosal carrier-delivery system for induction of peripheral immunological tolerance,” Proc. Natl. Acad. Sci. (USA) 91: 10795 (1994). The effective dosage range for non-cytokine synergists for mammals is from about 15 ng to about 15 mg per kg weight and preferably 300 ng-12 mg per kg weight. The effective dosage range for oral Type I interferon for mammals is from 1,000-150,000 units with no maximum effective dosage having been discerned. Another active compound that may be useful in combination with E-selectin is methotrexate which is known to cause a marked Th2 immune deviation with greatly increased IL-4 secretion when given on a pulse regimen (Weiner et al., “Treatment of Autoimmune Disease Using Tolerization in Combination with Methotrexate,” U.S. Pat. No. 5,935,577 (1999).

Ascertaining the optimum regimen for administering E-selectin and/or the co-stimulatory molecule is determined in light of the information disclosed herein and well known information concerning administration of bystander antigens and autoantigens. Routine variation of dosages, combinations, and duration of treatment is performed under circumstances wherein the effects of such variations on the organism can be measured. The co-stimulatory agent is preferably administered within 24 hours of administration of E-selectin. More preferably, it is administered at the same time as E-selectin. Most preferably, both are administered in a combined oral formulation.

The following examples describe and illustrate the methods and compositions of the invention. These examples are intended to be merely illustrative of the present invention, and not limiting thereof in either scope or spirit. Those of skill in the art will readily understand that variations of the materials used in, and the conditions and processes of, the procedures described in these examples can be used.

### Example 1

### Reduction of Brain Infarcts by Administration of E-Selectin

This Example illustrates the effects of administering E-selectin on reducing the incidence and size of infarcts in the brains of stroke-prone rats. Further information on stroke treatment by E-selectin tolerization can be obtained in a related application, PCT Application Ser. No. PCT/US01/16583, which is incorporated by reference herein in its entirety.

### Materials and Methods

Male and female stroke-prone and spontaneously hypertensive (SHR-SP) 8-10 week-old rats were obtained from the NIH colony. Okamoto (1974) Circ. Res. (Suppl.) 34, 35: 1 (1974). At 11 weeks of age, soluble human E-selectin (encoding the following domains: human E-selectin lectin, EGF, CR1, CR2 with a myc peptide tail), ovalbumin or vehicle (PBS) were administered intranasally. Purified human E-selectin was obtained from Protein Design Laboratories (Fremont, Calif.).

E-selectin and control preparations were administered in the following manner: SHR-SP rats were divided into three groups: (1) a saline (PBS) control group, (2) an E-selectin administration group (ES group), and (3) an ovalbumin (OVA) administration group (OVA group). In addition, ES and OVA groups were divided into single (non-booster) and repetitive (booster) administration groups. For the control group, 20 μl of phosphate-buffered saline (PBS) was administered into each nostril every other day for 10 days for a total of 5 administrations. For the ES non-booster group, 2.5 μg E-selectin in 20 μl PBS was administered into each nostril every other day for 10 days for a total of 5 administrations. For the ES booster group, an initial 2.5 μg of E-selectin in 20 μl PBS was administered as above for the non-booster group; additionally, 2.5 μg of E-selectin in 20 μl of PBS was administered intranasally into each nostril every other day for 10 days (3 weeks after the first E-selectin course) and repeated every 3 weeks until the animals were sacrificed. For the OVA non-booster group, 2.5 μg ovalbumin in 20 μl PBS was administered into each nostril every other day for 10 days for a total of 5 administrations. For the OVA booster group, an initial 2.5 μg of ovalbumin in 20 μl PBS was administered into each nostril as above for the non-booster group; additionally, 2.5 μg of ovalbumin in 20 μl of PBS was administered intranasally into each nostril every other day for 10 days (3 weeks after the first ovalbumin course) and repeated every 3 weeks until the animals were sacrificed.

The rats were evaluated for physical and neurological signs of stroke. These evaluations included an assessment of excitement (i.e., piloerection, hyperkinesis), hyperirratibility (i.e., jumping, trying to escape), behavioral and psychological depression (i.e., hypokinesis, hyposthenia, hyporesponsiveness), motion disturbance (i.e., transient episode of repetitive lifting of paws, ataxia, paresis, paralysis), and late symptoms observed near the time of death (i.e., apathy, coma, urinary incontinence). The rats were also monitored by measuring arterial blood pressure, body weight, heart weight, and arterial blood gas using methods available in the art.

Infarcts were evaluated in the following manner. When animals showed signs of cardiac failure, kidney failure, or stroke, they were perfused and their brains were removed for histology and image processing. Sections from 8 predetermined stereotactic levels were cut from each brain (total of 240 sections). The number and area of infarcts or hemorrhages were determined for each section from each animal. Statistical significance of E-selectin administrations was determined by comparing E-selectin groups to control groups by a Cox Proportional Hazards Model.

The animals lived for variable periods from 14 weeks to the termination of the experiment at 56 weeks. Deaths were caused by heart failure and kidney failure secondary to severe hypertension (mean systolic blood pressure 215 mm Hg), as well as by strokes. Average age at time of death and average systolic blood pressure did not differ among the experimental groups.

### Results

The experimental group of animals that received E-selectin with booster administrations had a statistically significant reduction in the frequency and area of infarcts compared to control groups (p<0.0001). Mean area of infarcts decreased from between about 6.873 mm² to about 27.718 mm² in control and single administration E-selectin groups to about 0.002 mm² in the E-selectin booster group (i.e., a greater than 99% reduction; see Tables I-IV). Mean number of infarcts decreased from about 3.0 to about 7.3 for control and single administration E-selectin groups to about 0.3 in E-selectin booster groups (i.e., a greater than 91% reduction; see Tables I-IV). Intraparenchymal hemorrhages were absent from the E-selectin booster group, but were present at an average number of from about 3.2 to about 2.3 per brain section analyzed in control and single E-selectin administration groups (see Tables I-IV).

TABLE I
Group OVA Data

| | | |
|---|---|---|
| | | Intraparenchymal |
| Sample | Infarcts | Hemorrhage |

| (sex) | Number | Area (mm²) | Number | Area (mm²) |
|---|---|---|---|---|
| 1 (female) | 13 | 6.966 | 2 | 0.439 |
| 2 (female) | 0 | 0 | 0 | 0 |
| 3 (female) | 1 | 0.062 | 15 | 0.390 |
| 4 (female) | 19 | 133.850 | 4 | 0.950 |
| 5 (female) | 15 | 70.559 | 1 | 0.02 |
| 6 (female) | 10 | 10.308 | 0 | 0 |
| 7 (female) | 0 | 0 | 0 | 0 |
| 8 (female) | 0 | 0 | 0 | 0 |
| mean | 7.3 | 27.718 | 2.8 | 2.25 |

TABLE II
Group OVAb Data

| | | |
|---|---|---|
| | | Intraparenchymal |
| Sample | Infarcts | Hemorrhage |

| (sex) | Number | Area (mm²) | Number | Area (mm²) |
|---|---|---|---|---|
| 1 (female) | 0 | 0 | 0 | 0 |
| 2 (female) | 3 | 0.734 | 1 | 4.784 |
| 3 (female) | 21 | 40.502 | 17 | 1.372 |
| 4 (female) | 0 | 0 | 0 | 0 |
| 5 (female) | 0 | 0 | 1 | 0.063 |
| 6 (female) | 0 | 0 | 0 | 0 |
| mean | 4.0 | 6.873 | 3.2 | 1.037 |

TABLE III
Group ES Data

| | | |
|---|---|---|
| | | Intraparenchymal |
| Sample | Infarcts | Hemorrhage |

| (sex) | Number | Area (mm²) | Number | Area (mm²) |
|---|---|---|---|---|
| 1 (female) | 0 | 0 | 0 | 0 |
| 2 (male) | 0 | 0 | 0 | 0 |
| 3 (female) | 9 | 13.488 | 5 | 0.177 |
| 4 (female) | 14 | 77.909 | 13 | 7.553 |
| 5 (female) | 0 | 0 | 0 | 0 |
| 6 (female) | 1 | 0.012 | 0 | 0 |
| 7 (male) | 0 | 0 | 0 | 0 |
| 8 (male) | 0 | 0 | 0 | 0 |
| mean | 3.0 | 11.426 | 2.3 | 0.966 |

TABLE IV
Group ESb Data

| | | |
|---|---|---|
| | | Intraparenchymal |
| Sample | Infarcts | Hemorrhage |

| (sex) | Number | Area (mm²) | Number | Area (mm²) |
|---|---|---|---|---|
| 1 (male) | 0 | 0 | 0 | 0 |
| 2 (female) | 0 | 0 | 0 | 0 |
| 3 (female) | 0 | 0 | 0 | 0 |
| 4 (female) | 0 | 0 | 0 | 0 |
| 5 (male) | 1 | 0.003 | 0 | 0 |
| 6 (female) | 0 | 0 | 0 | 0 |
| 7 (female) | 1 | 0.011 | 0 | 0 |
| 8 (male) | 0 | 0 | 0 | 0 |
| mean | 0.3 | 0.002 | 0 | 0 |

### Example 2

### Induction of Tolerance to E-Selectin

This Example provides data showing that tolerance to E-selectin was induced by the intranasal administration protocol of E-selectin described above, which resulted in decreased stroke-related tissue damage.

For this analysis, either E-selectin or control PBS preparations were administered to rats as described in Example 1 for the non-booster groups. Thus, 2.5 μg E-selectin in 20 μl PBS was administered into each nostril every other day for 10 days for a total of 5 administrations.

Fourteen days after intranasal administration to induce tolerization, delayed-type hypersensitivity (DTH) was analyzed by injecting 5 μg of E-selectin in 50 μl of PBS and 50 μl of complete Freund's adjuvant into hindpads (s.q.). Another fourteen days later, the rats were re-challenged by injecting 5 μg E-selectin in 50 μl PBS into the ear. Ear thickness was measured with microcalipers (Mitsutoyo) 48 hours later to assess to degree of tolerization to E-selectin.

Results of the delayed-type hypersensitivity assay demonstrated that intranasal instillation of human E-selectin induced tolerance. Administration of E-selectin intranasally before footpad and ear injection resulted in a significant suppression of ear swelling compared to control groups, as measured with Mitsutoyo microcalipers. In particular, rats treated with PBS exhibited an approximate 55% change in ear thickness (about 0.36 mm swelling), while the E-selectin treated rats exhibited only about a 20% change in ear thickness (about 0.11 mm swelling). The difference was statistically significant at the p<0.01 level.

These data demonstrate that the E-selectin administration protocol used induced tolerance to E-selectin.

### Example 3

### Vascular Dementia Animal Model

This Example provides information about the animal model used for evaluation of vascular dementia and the effects of E-selectin tolerization on vascular dementia.

The experimental model used for vascular dementia was hypoperfusion of Wistar rat brains. In particular, previous work has shown that cerebrovascular white matter lesions can be experimentally induced in the rat brain as a result of chronic cerebral hypoperfusion and that such hypoperfusion leads to impaired memory. See Sarti et al., Persistent impairment of gait performances and working memory after bilateral common carotid artery occlusion in the adult Wistar rat, BEHAVIORAL BRAIN RESEARCH 136: 13-20 (2002). This model is created by permanent occlusion of both common carotid arteries as described below.

The animals were anesthetized with 5 isoflurane for induction and 1.5% isoflurane for maintenance in 30% O₂/70% N₂O by facemask. The core body temperature was monitored and maintained at 37.0±0.5° C. using a heating pad and a heating lamp. Through a midline cervical incision, both common carotid arteries were exposed and double-ligated with 5-0 silk sutures as previously described by Wakita H., Acta Neuropathol. (Berl) 1994; 87: 484-492. After the operation, the rats were kept in cages with food and water ad libitum. As controls, four animals were subjected to the same surgical procedures without bilateral carotid ligation.

Cerebral blood flow (CBF) after carotid artery occlusion was 30 to 50% of the control several days after ligation. The CBF decreased to values ranging from 33 to 80% of control over a prolonged period (1 week-1 month).

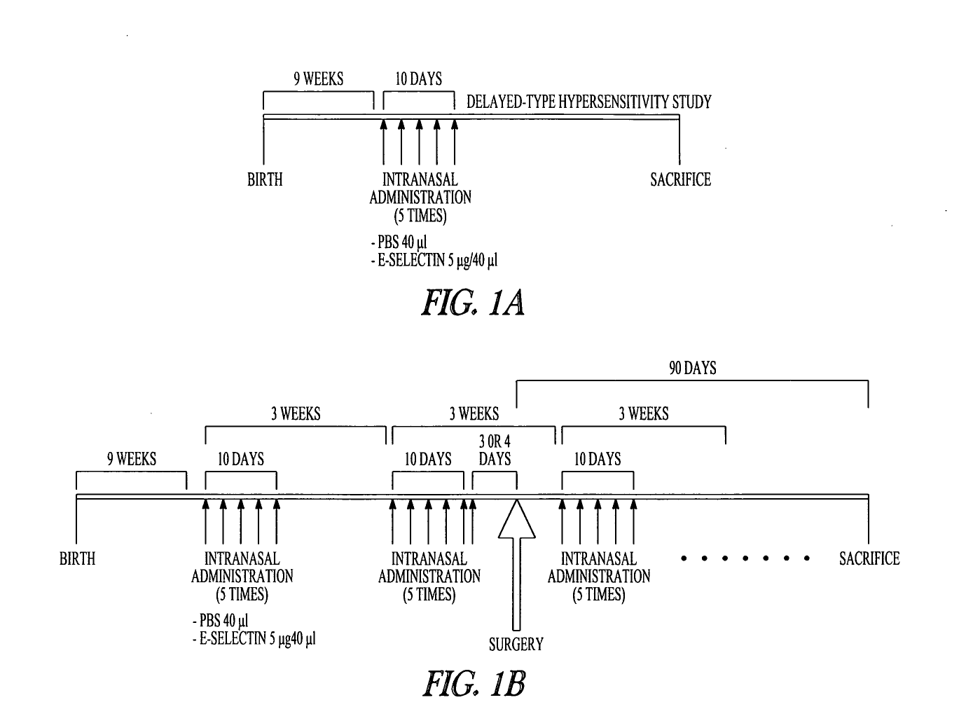

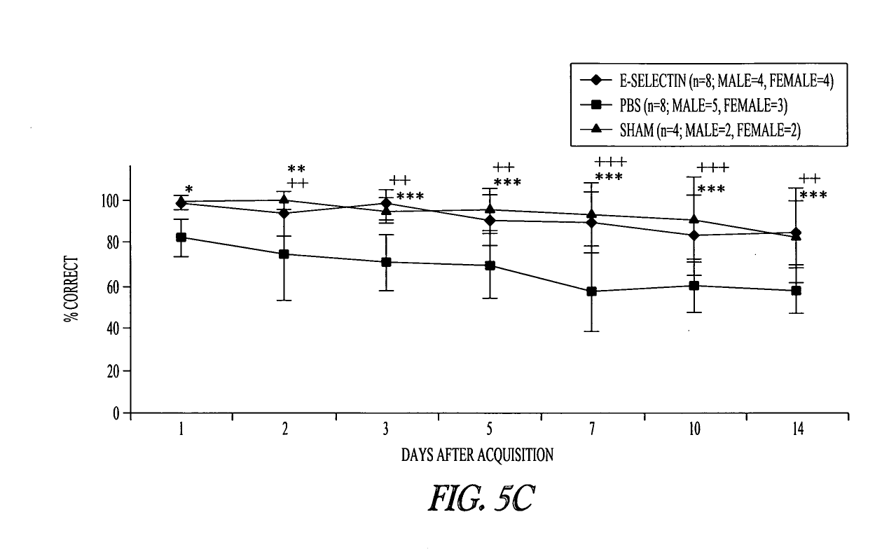

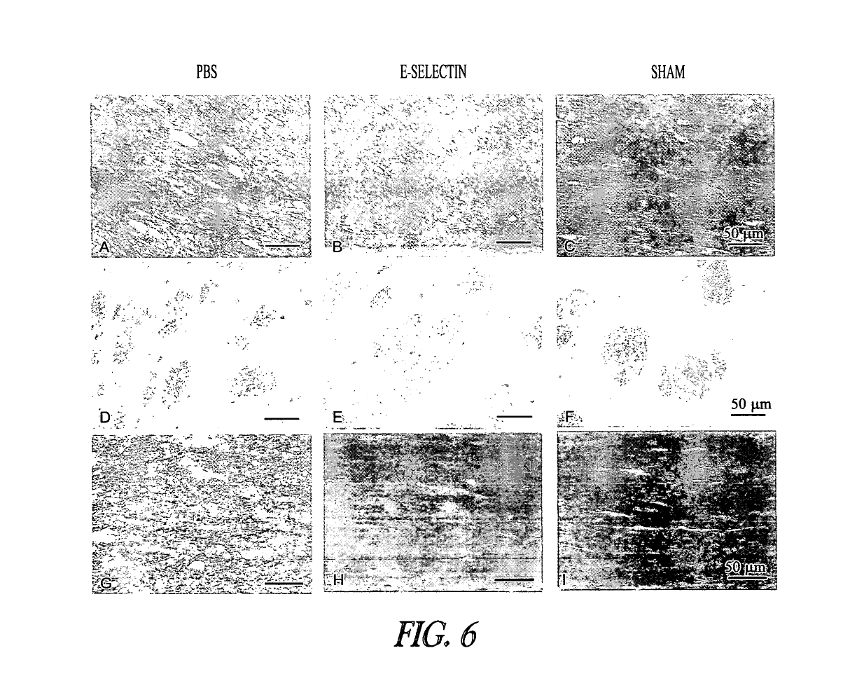

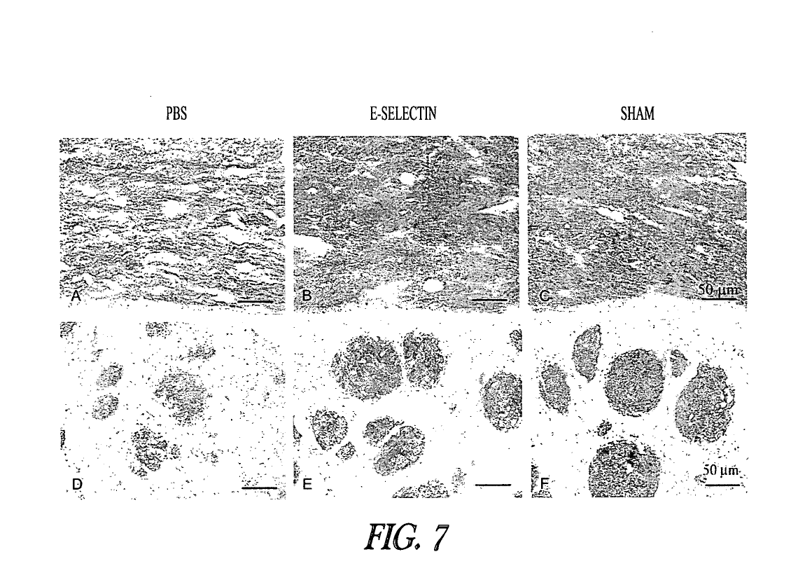

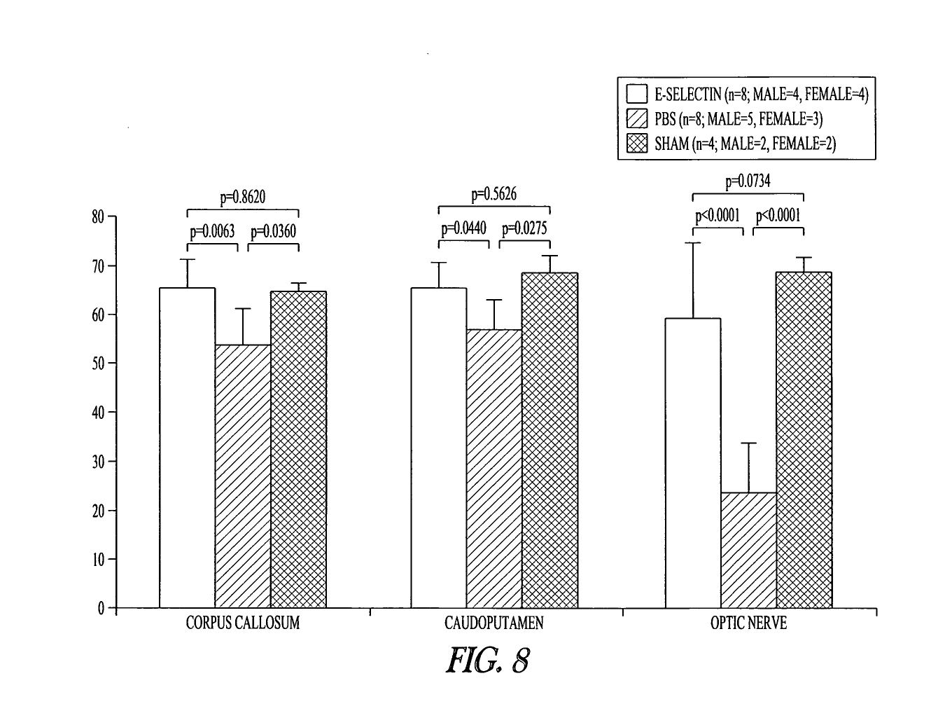

The effects of carotid artery occlusion upon brain tissues are illustrated in FIGS. 6-13 by comparing results for the PBS treated animals, who received bilateral carotid ligation after administration of PBS during the tolerization schedule (shown in FIG. 1), with the sham-operated animals that did not receive bilateral carotid ligation. As shown FIGS. 6-8, white matter becomes rarefied after carotid artery occlusion (compare PBS vs. Sham-operated tissue sections in FIG. 6-7 and graphic summary in FIG. 8).

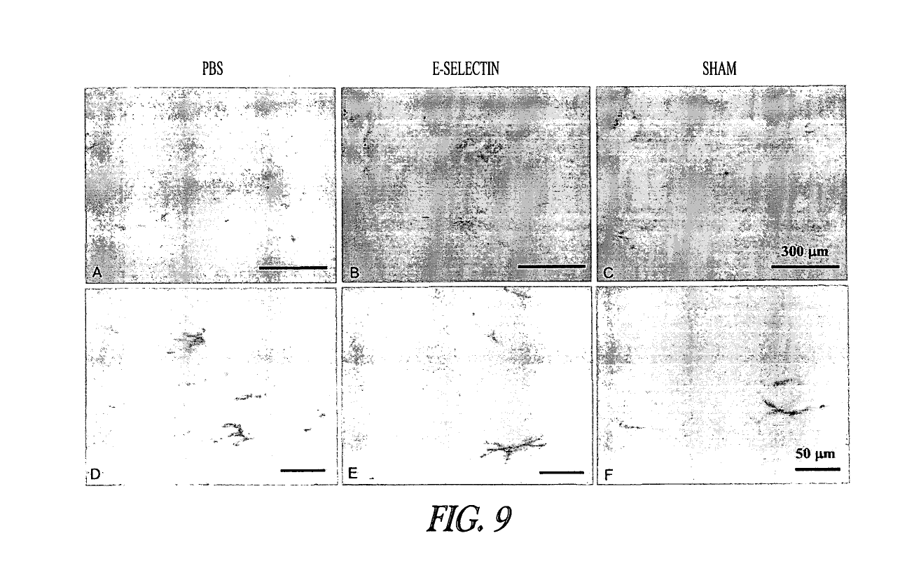

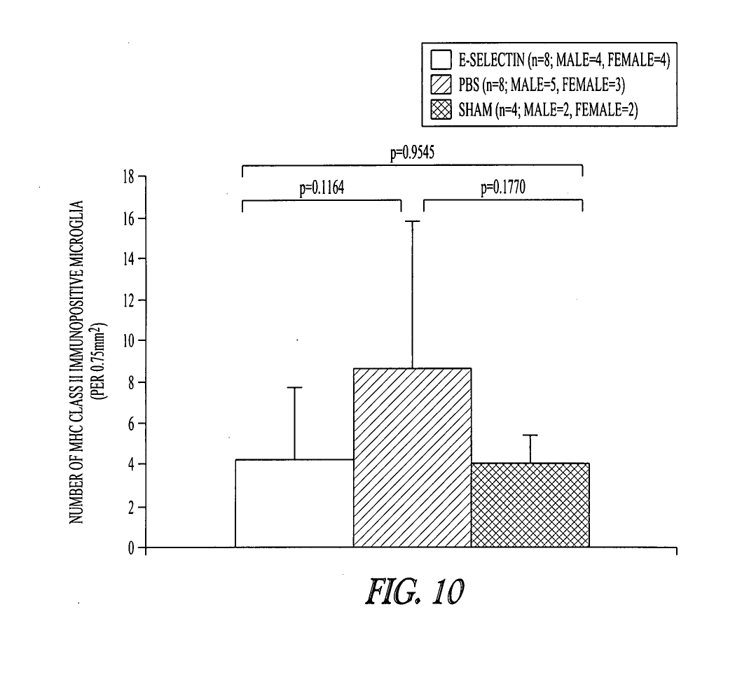

FIG. 9 shows that glial cells become activated in white matter after carotid artery occlusion. In particular, as summarized in FIG. 10, a trend toward (p=0.177) greater numbers of MHC class II immunopositive microglia are observed in rats who received bilateral carotid ligation (“PBS” rats) than in rats that did not receive bilateral carotid ligation (“Sham” operated rates). Thus, microglia and astroglia were activated briefly after vascular occlusion. Moreover, such activation was predictive of the extent and severity of the subsequent white matter damage.

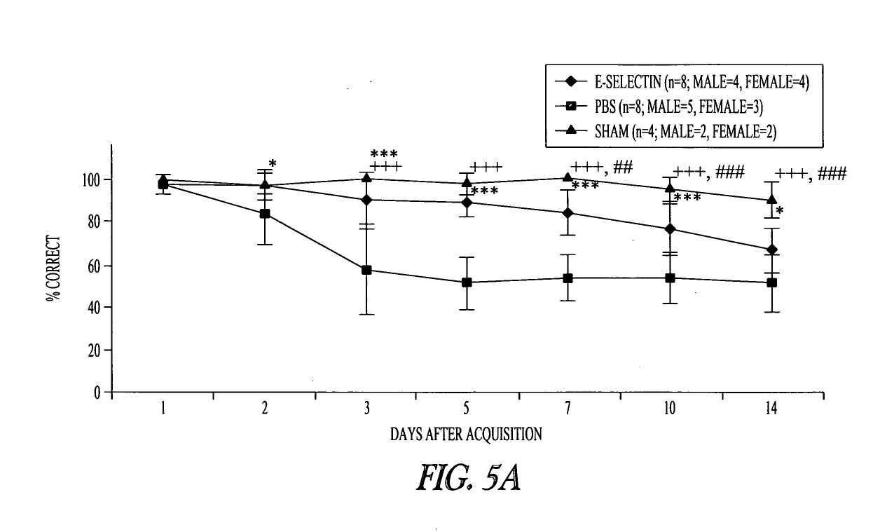

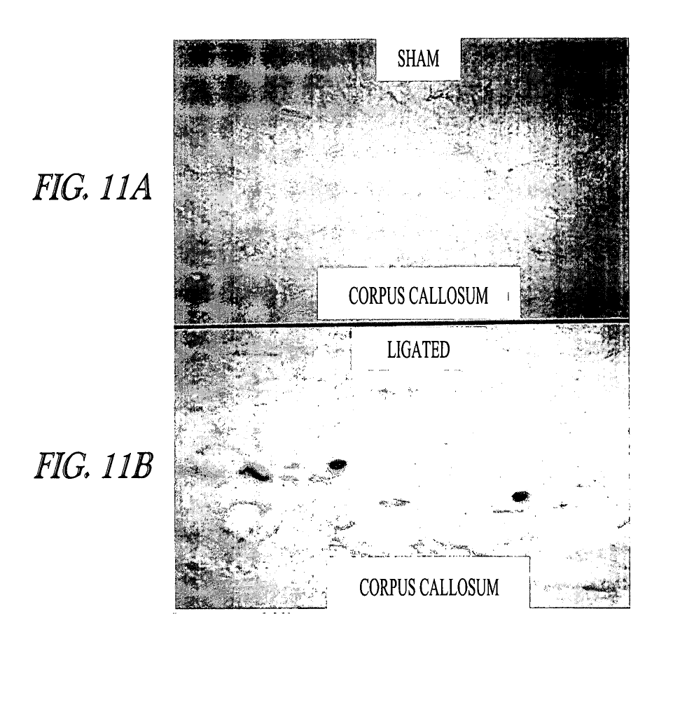

Additional lymphocytes were detected with CD4 or CD8 antibodies (FIG. 4) after occlusion of carotid arteries. As shown in FIG. 11, greater numbers of CD4 positive T cells infiltrated the corpus callosum of rats who received bilateral carotid ligation (“Ligated” rats) than was observed in rats that did not receive bilateral carotid ligation (“Sham” operated rats). These CD4 or CD8 positive T cells were scattered in the white matter after occlusion. These changes are similar to those in human cerebrovascular white matter lesions and suggest that inflammatory and immunologic reactions play a role in the pathogenesis of the white matter changes.

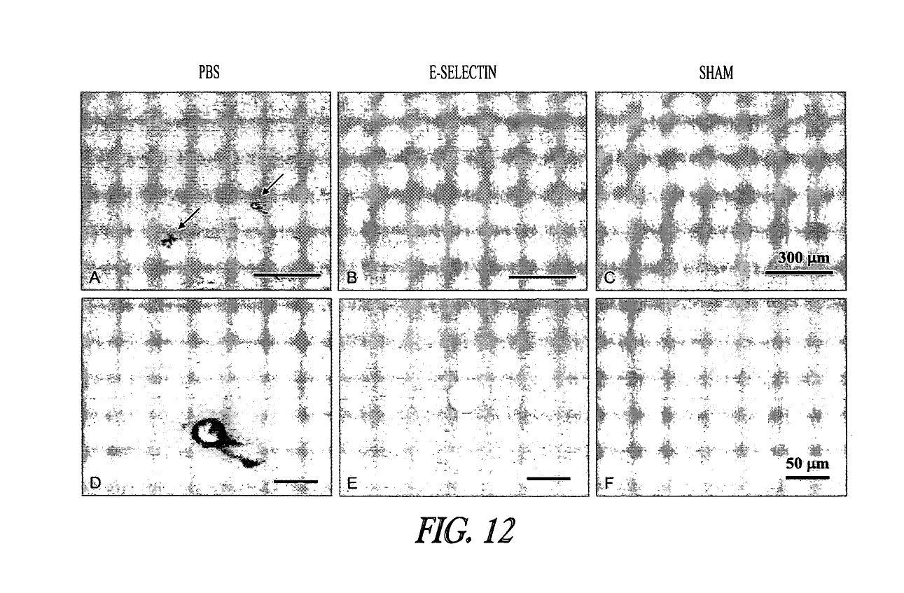

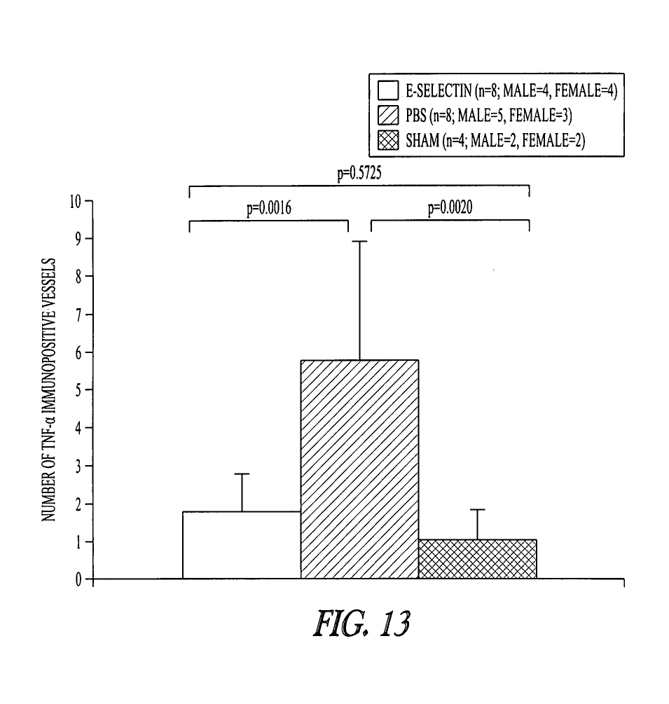

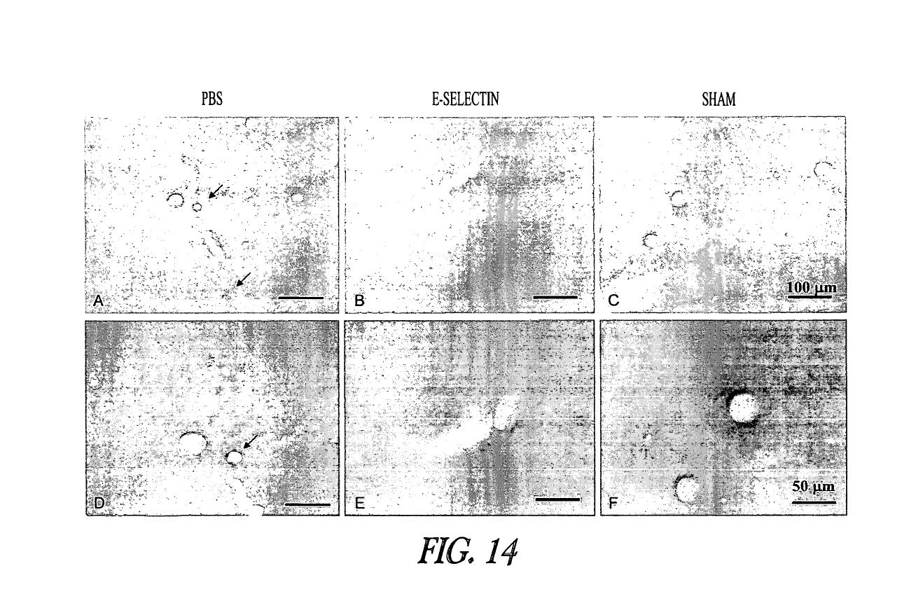

FIG. 12 show that the numbers of TNF-α immunopositive blood vessels increase after carotid artery occlusion. In particular, as summarized in FIG. 13, significantly (p=0.002) greater numbers of TNF-α immunopositive blood vessels are observed in rats who received bilateral carotid ligation (“PBS” rats) than in rats that did not receive bilateral carotid ligation (“Sham” operated rats) or rats subjected to ischemia after E-selectin tolerization.

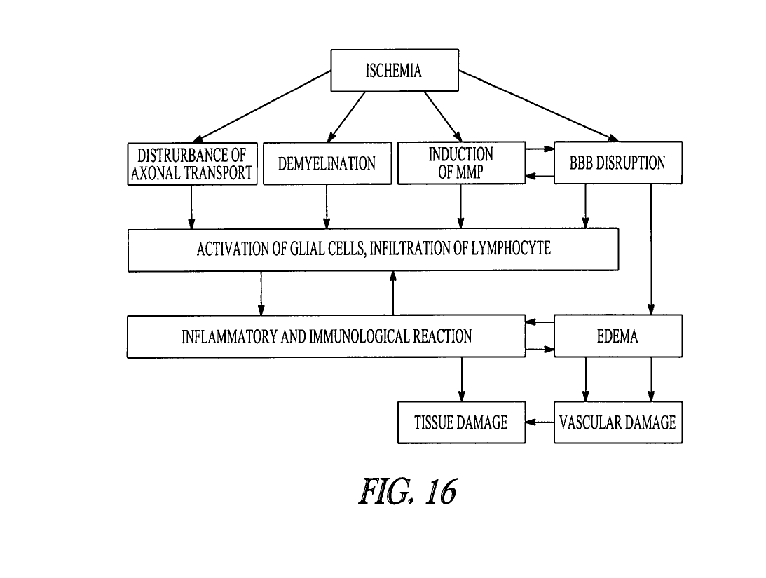

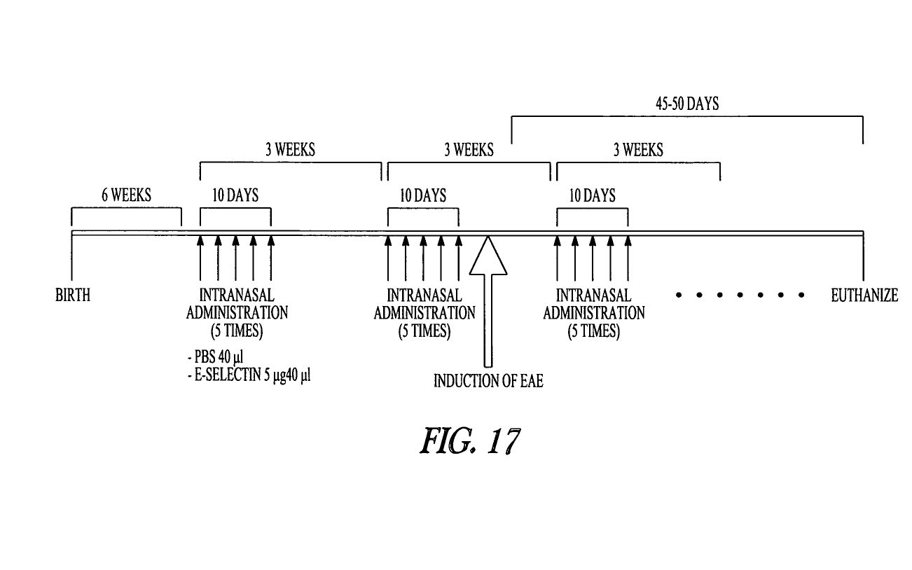

These data indicate that immunological activity accompanies brain damage after carotid artery occlusion. The effects of carotid artery occlusion (ischemia) upon brain function are summarized in FIG. 16.

### Example 4

### E-Selectin Administration Ameliorates Vascular Dementia

This Example illustrates that mucosal tolerization to E-selectin protects against several forms of memory dysfunction and white matter damage in the rat model of vascular cognitive impairment.

### Materials and Methods

Animals: A total of 34 Male and female Wistar rats (Charles River Laboratories, Wilmington, Mass., USA) aged 9 weeks were used. The National Institute of Neurological Disorders and Stroke Animal Care and Use Committee approved all experiments.

Tolerization Schedule Animals were divided into two groups. Intranasal application of E-selectin was carried out with the animals under brief anesthesia with 5% isoflurane in 30% O₂/70% N₂O.

Intranasal instillations to animals in groups 1 and 2 were as follows:

(1) Control rats received PBS (Quality biological, Inc, Gaithersburg Md., USA)

(2) Experimental rats received recombinant human E-selectin (Novavax, Rockville Md., USA)

The administration schedule involved a single series of administrations or a single series of administrations plus a booster series of administrations as follows (see FIG. 1):

(1) Single or non-booster administration schedule: PBS (20 or E-selectin (2.5 μg/20 μl) was instilled into each nostril every other day for 10 days (total of 5 administrations) (FIG. 1).

(2) Booster administration schedule: intranasal instillations of the same substance at the same volume and concentration on the same schedule as described for the single or non-booster schedule described above, but the administrations were repeated at 3-week intervals from 1 month before surgery to 3 months after surgery (FIG. 1).

Delayed-Type Hypersensitivity Reaction: For assessing the delayed-type hypersensitivity reaction, a single-course tolerization schedule with either PBS or E-selectin was conducted (n=4) (as shown in FIG. 1 for the non-booster group). Fourteen days later, the animals were immunized (hind footpad) with 75 μg E-selectin/200 μl PBS plus 50 μl complete Freund's adjuvant (Sigma, St. Louis, Mo.). Fourteen days after that, ear thickness was measured and the animals were re-challenged with 75 μg E-selectin/100 μl PBS injected into the ear. Ear thickness increase over baseline was measured with microcalipers (Mitsutoyo Co, Ltd, Kawasaki, Kanagawa, Japan) 2 days later.

Surgery: The booster tolerization schedule was repeated at 3-week intervals from 1 month before the surgery to 3 months after the surgery. In order to adjust the surgery workload, half of the rats from each group were randomly selected and subjected to the surgery 3 days after the last dose of the first booster tolerization schedule; and surgery was performed 4 days after the last dose of the first booster tolerization schedule for the remaining half of rats.

The animals were anesthetized with 5% isoflurane for induction and 1.5% isoflurane for maintenance in 30% O₂/70% N₂O by facemask. The core body temperature was monitored and maintained at 37.0±0.5° C. using a heating pad and a heating lamp. Through a midline cervical incision, both common carotid arteries were exposed and double-ligated with 5-0 silk sutures as previously described by Wakita H., Acta Neuropathol. (Berl) 1994; 87: 484-492. After the operation, the rats were kept in cages with food and water ad libitum. As controls, four animals were subjected to the same surgical procedures without bilateral carotid ligation.

Behavioral assessment: Behavioral assessment consisted of object recognition, T-maze spontaneous alternation, and T-maze left/right discrimination memory retention tests. An observer who was blind to the treatments performed behavioral assessment.

Object Recognition test: This test evaluates non-spatial working memory related to the frontal subcortical circuits (Ennaceur A. Behav Brain Res 1988; 31:47-59, Sarti C, Behav Brain Res. 2002; 136:13-20). The apparatus was formed by a glass box (30×60×30 cm). The apparatus was illuminated by a 100 W lamp suspended 70 cm above the box in a darkened room. The day before testing, rats were habituated to the test environment by exploring the box for 6 min without objects. On the day of the test, a session of two trials was given. The inter-trial interval was 60 min.

In the first trial, two identical objects were placed on the centerline of the long axis of the floor, 5 cm from each end of the apparatus. Rats were placed into the center of the box and allowed to explore the two objects for 6 min. The amount of time spent exploring each object was recorded. During the second trial, one of the objects presented in the first trial is replaced by a novel object and rats are left in the box for 6 min. The time spent for the exploration of the familiar (Tf) and the novel object (Tn) is recorded separately. Exploration is considered sniffing at the object within a distance of 2 cm from the object and/or touching it with the nose. A discrimination index (Tn-Tf/Tn+Tf) is calculated.

For each animal, one pair of objects in the first trial was selected at random from a set of three plastic objects that differed in shape and color (red cubes, green pyramids, and blue cylinders of 6 cm height), and the role (familiar and novel object) and the position of the two objects in the second trial were randomly changed to avoid object and place preference. After each exposure, the apparatus and the objects were cleaned carefully with 70% alcohol to avoid olfactory stimuli.

T-maze spontaneous alternation: This test evaluates spatial working memory related to the frontal subcortical circuits (Bartolini L., Pharmacol Biochem Behav. 1992; 43:1161-1164, Sarti C, Behav Brain Res. 2002; 136:13-20). Spontaneous alternation was investigated in an acrylic T shaped runway. It consisted of a start box (20×18 cm) and start arm (60 cm long), and two identical goal arms (both 50 cm long). All arms were 10 cm wide and 10 cm high. Spontaneous alternation refers to the instinctive behavioral tendency by which rats typically alternate their choices between the arms of the T-maze more often than they repeat their initial choice. Rats were placed in the start box of the T-maze and a maximum time of 5 min was allowed for them to explore the maze. Spontaneous alternation was defined as following: the rat entered with all four feet into one goal arm, came back, and then entered with all four feet into the opposite goal arm. The number of rats who alternated was recorded.

T-maze left/right discrimination memory retention: This test evaluates spatial reference memory related to the hippocampus and caudoputamen (Oliveira MG, Neurobiol Learn Mem 1997; 68:32-41). This test was repeated at 2, 6 and 10 weeks after surgery. The dimensions of the T-maze apparatus were described above. The exit of the start box and the entrances of the goal arms could be blocked by guillotine doors. Careful consideration was given to avoid providing the animals with any spatial cues. To minimize olfactory cues, the maze was wiped carefully after each run with 70% alcohol.

Training sessions for left/right discrimination memory retention: The day before training, after the spontaneous alternation test, rats were habituated for 15 min to the presence of food pellets (Bacon Softies; Bio-Serv, Frenchtown, N.J., USA) placed at the end of each arm in the T-maze. On days 1 to 3, the rats were food-deprived for 8 to 12 hours each day before the T-maze left/right discrimination training. This training consisted of 3 stages. In the performance of the training, half of the rats from each group were randomly selected and reinforcement (food reward) placed on the right arm; for the other half of the rats from each group, the reinforcement was placed on the left arm. The reinforced arm then remained consistent throughout the training period. The first stage consisted of 5 trials. In this stage, a guillotine door was placed to close off one arm, and the animal was forced to enter the open arm, which was baited with a food reward that the animal was allowed to eat. For all runs the animals remained on the maze until 2 min had elapsed; they were then placed in the start box for 2 min. The second stage consisted of 5 trials. In this stage, a guillotine door was placed to close off the same arm as that in the first stage, and the animal was forced to enter the open arm, which was not baited with a food reward. When the animal entered into the open arm, a food reward was given and the animal was allowed to eat the food. The animals remained on the maze for 2 min and were then placed in the start box for 2 min. In the third stage, a guillotine door was removed, and the animal could enter into either arm (correct side and incorrect side). If the animal chose the arm on the correct side the animal received a food reward and was allowed to eat for 2 min after which it was placed in the start box for 2 min. If the animal chose the incorrect side-arm, the animal was picked up immediately and placed in the start box for 2 min. The third stage was continued until the animals made 4 consecutive correct choices or until they had had 20 training sessions (the training ceiling). This procedure was performed daily on three successive days (on days 1 to 3).

Left/right discrimination memory retention test session: The retention of left/right discrimination memory was evaluated at 1, 2, 3, 5, 7, 10 and 14 days after the training session. The animals were given 10 trials on each testing day. An entry was defined as all four paws entering the arm. The total number of correct entries was recorded.

Histopathology: At 90 days after surgery, the animals were deeply anesthetized with sodium pentobarbital (100 mg/kg, intraperitoneally), perfused transcardially with 0.01 M PBS, and then perfused with a fixative containing 4% paraformaldehyde in 0.1 M phosphate buffer (PB, pH 7.4). Coronal brain blocks including the caudoputamen or optic nerve were embedded in paraffin for histological examination. Two micrometer-thick paraffin sections were then cut on a microtome. The luxol fast blue stain was used to evaluate the myelin damage. Immunocytochemistry with the cocktail of monoclonal antibodies directed against non-phosphorylated neurofilaments (SMI 311, Covance Research Products, Inc., Berkeley, Calif., USA) was used for the assessment of axonal injury (Rosenfeld J, J Neuropathol Exp Neurol. 46:269-282 (1987)). The sections were incubated for 1 hr in 0.1 M PBS containing 0.3% Triton X-100 for permeabilisation. Ten percent donkey serum was applied for blocking followed by incubation overnight in primary antibody (SMI 311) in a dilution of 1:500. The sections were subsequently incubated with a biotinylated anti-mouse IgG raised in donkey (Jackson Immuno Research Labs, West Grove, Pa., USA, 1:2000) for 1 hr, and then incubated with an avidin-biotin peroxidase complex solution (Vector Laboratories, Burlingame, Calif., USA, 1:100) for 1 hr. After each incubation, the sections were rinsed for 30 min with 0.1 M PBS containing 0.3% Triton X-100. The immunoreaction products were visualized with diaminobenzidine (DAB kit, Vector Laboratories, Burlingame, Calif., USA). The severity of the white matter lesions was evaluated by the fiber density of luxol fast blue-stained sections. Monochromatic photo images of both sides of the corpus callosum, the traversing fiber bundles of the caudoputamen bilaterally and both optic nerves were taken by means of a microscope with a ×40 objective connected to a digital camera (MetaMorph Image Processing System, Universal Imaging Corp, Downingtown, Pa., USA). These images were converted into PICT files by Photoshop (Adobe Systems Incorporated, San Jose, Calif., USA) and the fiber density of each PICT file was analyzed with the NIH image computer program. To account for the variation of the fiber density between right and left sides of the corpus callosum and caudoputamen, the average of the fiber densities of both sides was calculated.

For free-floating immunohistochemistry, the rest of the coronal blocks were post-fixed for 12 hrs in 4% paraformaldehyde in 0.1 M PB (pH 7.4), and stored in 20% sucrose in 0.1 M PB (pH 7.4) until used. Serial sections (20 μm thick) were then cut on a cryostat. Endogenous peroxidase was inactivated by immersing the sections in a solution of 0.3% hydrogen peroxide in 10% methanol/0.1M PBS for 30 min. To block nonspecific staining, sections were incubated in 5% normal horse serum in 0.1 M PBS containing 0.3% Triton X-100 for 1 hr. After blocking, the sections were incubated overnight with the following antibodies (mouse or goat anti-rat) (dilutions in parentheses): against the major histocompatibility complex (MHC) class II (Ia) antigen (OX 6, Serotec, Raleigh, N.C., USA, 1:100), against TNF (YC032, Yanaihara Institute, Fujinomiya, Shizuoka, Japan 1:800) and against E-selectin (R and D systems, Minneapolis, Minn., USA 50 μg/ml). The sections were subsequently incubated with a biotinylated anti-mouse IgG or a biotinylated anti-goat IgG (Vector Laboratories, Burlingame, Calif., USA, 1:200) for 1 hr, and then incubated with an avidin-biotin peroxidase complex solution (Vector Laboratories, Burlingame, Calif., USA, 1:100) for 1 hr. After each incubation other than that for blocking nonspecific staining the sections were rinsed for 15 min with 0.1 M PBS containing 0.3% Triton X-100. Finally, the immunoreaction products were visualized with diaminobenzidine (DAB kit, Vector Laboratories, Burlingame, Calif., USA). For assessment of nonspecific staining, primary antibodies were replaced with normal mouse or goat IgG. We counted the numerical density of the MHC class II (Ia) antigen immunopositive microglia/macrophages in a 0.75 mm² area in the corpus callosum and the number of TNF or E-selectin immunopositive vessels in the total area of the corpus callosum in a section at a level of −2.3 mm from bregma. To evaluate the hippocampal damage, 20 micrometer-thick frozen sections including hippocampus were stained with cresyl violet.

Immunoassay: The level of plasma TNF concentration was measured by a Rat TNF US ELISA kit (BioSource International, Camarillo, Calif., USA) following the manufacturer's instructions. The O.D. values (450 nm) were measured by SpectraMax M5 (Molecular Devices, Sunnyvale, Calif., USA) and the concentration of the plasma TNF was calculated.

Statistical analysis: Data are represented as mean±SD. Differences in the mortality rates between groups were determined by Fisher's exact probability test.

Differences in the change of ear thickness between the groups were determined by unpaired Student's t-test. Differences in proportions of the T maze spontaneous alternation among each of the three groups were determined by χ² test. Differences in the discrimination index of the object recognition test and the percentages of correct arm entries on the T-maze left/right discrimination memory retention test among the groups were determined by repeated measure analysis of variance (ANOVA) followed by post-hoc testing with Fisher's protected least significant difference procedure. Differences in the fiber densities were determined by two-factor ANOVA followed by Fisher's protected least significant difference post-hoc testing. Differences in the numerical densities of either the MI-IC class II antigen immunoreactive microglia/macrophages, the TNF immunoreactive vessels or E-selectin immunoreactive vessels and in the level of plasma TNF concentration were determined by one-factor ANOVA followed by Fisher's protected least significant difference post-hoc testing. To evaluate the possible effect of optic nerve damage on the object recognition test, a Pearson correlation coefficient was calculated between the fiber density of the optic nerve and discrimination index in the E-selectin treated animals. p<0.05 was considered significant.

### Results

Mortality rates: None of the sham-operated animals died. Of the 11 animals that received E-selectin, 2 animals (18.2%) died within 7 days after surgery, one animal (9.1%) died by the anesthesia for the nasal instillation of E-selectin at 9 weeks after surgery. Of the 11 animals that received PBS, 4 animals (27.3%) died within 7 days after surgery. There was no significant difference in the mortality rates between the E-selectin and PBS groups.

Cerebral blood flow (CBF) without E-Selectin tolerization: Cerebral blood flow (CBF) was 30 to 50% of the control several days after ligation. The CBF decreased to values ranging from 33 to 80% of control over a prolonged period (1 week-1 month).

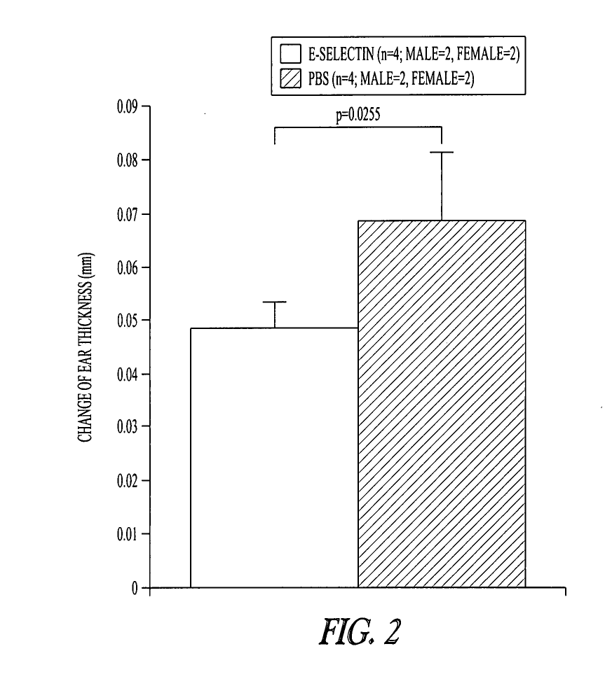

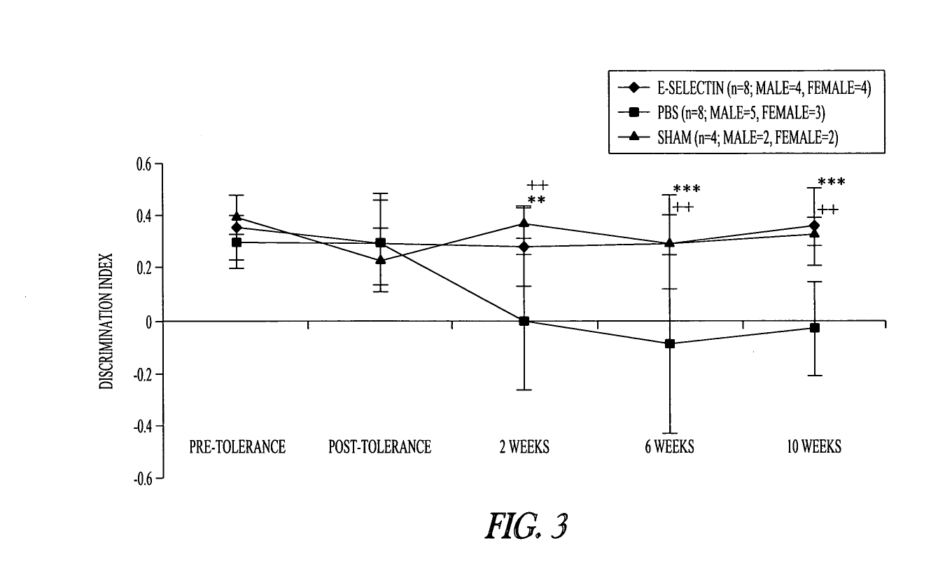

Delayed-type hypersensitivity after E-selectin treatment: A single course of tolerization with E-selectin significantly suppressed the ear swelling in the delayed-type hypersensitivity study (p=0.0255). FIG. 2 shows that rats treated with E-selectin had an ear thickness of slightly less than 0.05 mm whereas control rats that received only PBS had an ear thickness of almost 0.07 mm. These data indicate that rats treated with E-selectin became tolerant to later E-selectin administration in the ear flap and therefore did not exhibit as much inflammation and swelling.

### Behavioral Assessment

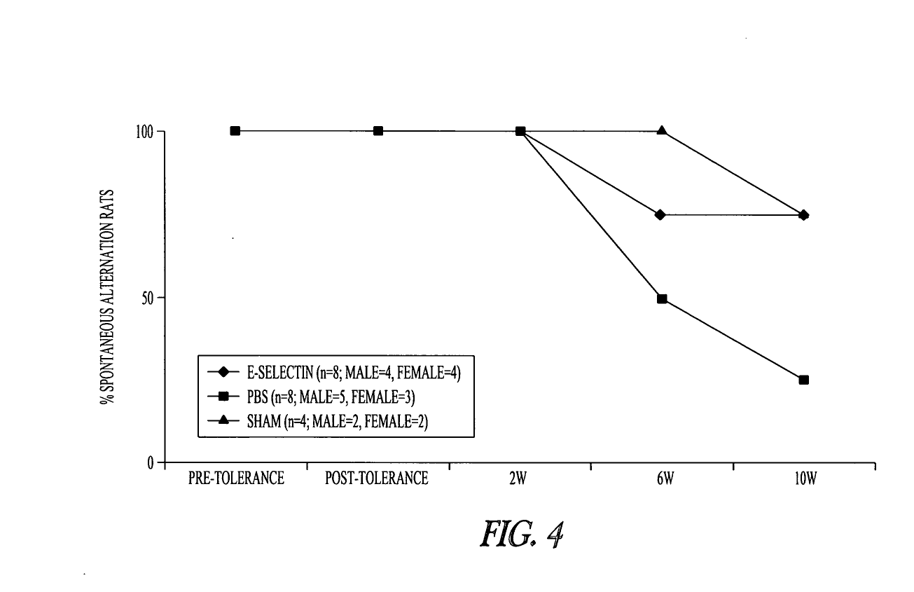

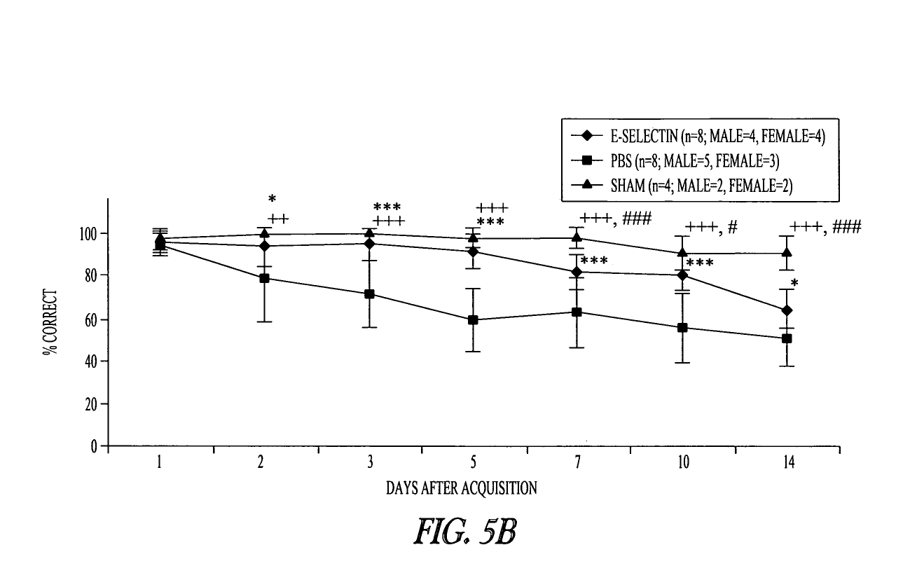

As described in more detail below, tolerization with E-selectin significantly improved the learning and memory impairment in the object recognition test (FIG. 3), T-maze memory retention (FIG. 5) and the ability to handle changes in the T-maze (FIG. 4), compared with the control group of rats that received only PBS.

Neurological impairment without E-Selectin tolerization: Gait performance declined over time in comparison with baseline. At 60 and 90 days, bilateral common carotid artery occlusion rats showed decreased performances on object recognition and T maze spontaneous alternation test in comparison with sham-operated rats.

Object recognition test: There were no significant differences in the discrimination index among the E-selectin, PBS and sham groups before surgery (baseline).

After surgery, the PBS group developed a reduced discrimination index. In contrast, the discrimination indices of the E-selectin and sham groups were maintained at the same baseline levels throughout the experiment. The discrimination indices of the PBS group were significantly decreased as compared with the E-selectin and sham groups (p=0.0005, p=0.0059 respectively). There were no significant differences in the discrimination index between the E-selectin and the sham groups (p=0.7397). Thus, induction and maintenance of mucosal tolerance to E-selectin protected against the decrease in discrimination observed in the PBS group. (FIG. 3).

T-maze spontaneous alternation: There were no significant differences in the percentage of spontaneously alternating rats among the E-selectin, PBS and sham groups before surgery and at 2 and 6 weeks after surgery.

However, by 10 weeks after surgery, the percentage of spontaneously alternating rats in the PBS group was significantly decreased compared with the E-selectin group (p<0.05). Thus mucosal tolerization to E-selectin protected against loss of the spontaneous alternation tendency seen in PBS treated rats. (FIG. 4).

### T-Maze Left/Right Discrimination Memory Retention

Two weeks after surgery: The numbers of correct arm entries did not differ among the E-selectin, PBS and sham groups tested 1 day after the training session. The percentages of correct arm entries were greater than 95%. However, in the PBS group, the number of correct arm entries decreased over time, and the percentage of correct entries between 3 and 14 days after the training session were diminished to 50 to 60%, which is close to a random choice level. This suggests that animals in this group had lost their left/right discrimination memory. The decrease in the number of correct arm entries was less prominent in the E-selectin group, and rats treated with E-selectin had a statistically significantly higher number of correct entries than the PBS-treated animals by repeated measure ANOVA (p<0.0001). In contrast with the PBS and E-selectin groups, the sham group retained their left/right discrimination memory at the same level throughout the experiment. The difference between the sham and the PBS groups was statistically significant (p<0.0001), and the difference between the sham and the E-selectin groups was also statistically significant by repeated measure ANOVA (p=0.0040) (FIG. 5A).

Six weeks after surgery: The E-selectin group had a higher number of correct entries than the PBS group by repeated measure ANOVA (p=0.008) (FIG. 5B). However, the difference between sham and E-selectin groups was still statistically significant by repeated measure ANOVA (p=0.0030) (FIG. 5B). The difference between sham and PBS groups was also statistically significant (p<0.0001). Hence, at six weeks, E-selectin tolerization had not completely ameliorated the effects of carotid ligation.

Ten weeks after surgery: In contrast with 2 and 6 weeks after surgery, the sham and the E-selectin groups both retained their left/right discrimination memory throughout the experiment, and there were no significant differences in the number of correct entries between these two groups by repeated measure ANOVA (p=0.6256). The E-selectin and sham groups had a higher number of correct entries than the PBS group by repeated measure ANOVA (p=0.0003, and p=0.0026, respectively) (FIG. 5C). Thus, mucosal tolerance to E-selectin led recovery of the spatial reference memory in left/right discrimination tasks.

Histopathology: In the sham-operated animals, there was no detectable rarefaction in the white matter. However, rarefaction of the white matter was observed in the corpus callosum, in caudoputamen traversing fiber bundles and in the optic nerve in the PBS-treated rats. For these studies, luxol fast blue stain and immunocytochemistry with a cocktail of antibodies directed against nonphosphorylated neurofilaments (SMI 311) were used. The severity of the rarefaction was markedly attenuated in the animals treated with E-selectin (FIGS. 6, 7). In particular, the fiber densities in the E-selectin-treated animals were significantly higher than those in the PBS-treated group by two-factor ANOVA (p<0.0001). Moreover, there was no significant difference in the fiber densities between sham and E-selectin groups (p=0.2026) (FIG. 8). Thus, E-selectin mucosal tolerization had a protective effect against white matter rarefaction induced by protracted hypoperfusion.

The Pearson correlation coefficient between the fiber density of the optic nerve and the discrimination index in the E-selectin treated animals was minus 0.470. This correlation was not significantly different from 0 (p=0.2537). A few dark neurons were detected in the unilateral hippocampus of the three E-selectin-treated (27.3%), three PBS-treated animals (27.3%) and one sham-operated animal (25%). There were no obvious differences in the number of the dark neurons among three groups (data not shown).

In the white matter of the sham-operated animals, there was positive immunostaining for the MHC class II (Ia) antigen in only a few glial cells. However, the brains of the PBS-treated animals showed an increase in the number of microglia/macrophages that were immunolabeled for the MHC class II (Ia) antigen. These microglia and macrophages were observed in the white matter within the corpus callosum and caudoputamen. In contrast, in E-selectin-treated rats, the number of microglia/macrophages positively immunolabeled for MHC class II antigen tended to correlate with a tendency towards a decrease in the white matter lesions as compared to PBS-treated animals (FIG. 9). However, there were no significant differences among E-selectin-treated group, sham-operated group and PBS-treated group (FIG. 10).

While TNF-α was prominently expressed in endothelial cells in blood vessels of the white matter, such TNF-α expression was markedly attenuated in E-selectin-treated and sham-operated animals (FIG. 12). The TNF immunoreactive vessels were significantly (p=0.0016) decreased in number in the E-selectin-treated groups as compared to the PBS-treated group. In contrast, there were no significant differences in the number of TNF immunoreactive vessels between the sham and E-selectin groups (p=0.5725) (FIG. 13).

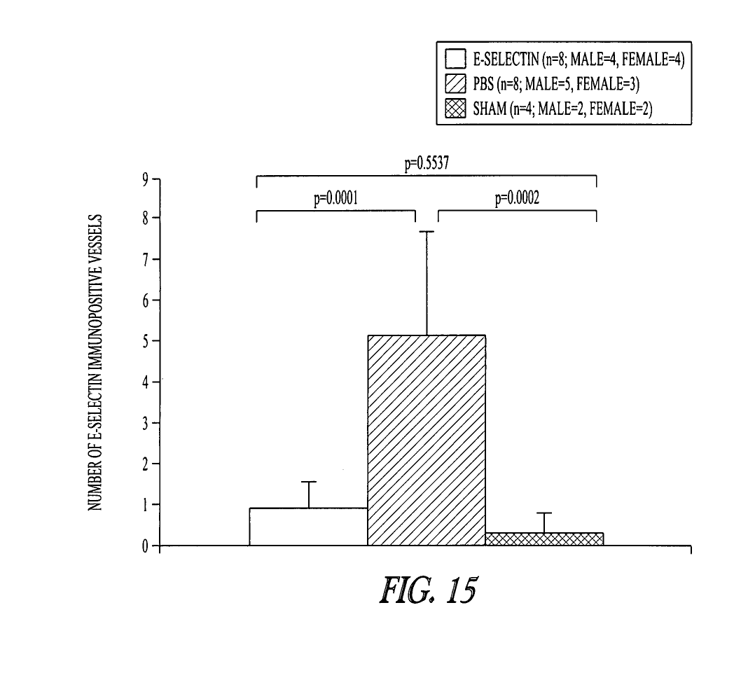

E-selectin was expressed in endothelial cells of vessels in the brains of the PBS-treated animals. The E-selectin immunoreactive vessels were decreased in number in the E-selectin-treated group as compared to the PBS-treated group (FIG. 14). The E-selectin-treated animals exhibited a significant reduction (p=0.0001) in the number of E-selectin immunopositive vessels as compared to the PBS-treated group. In contrast, there were no significant differences in the number of E-selectin immunoreactive vessels between sham and the E-selectin groups (p=0.5537) (FIG. 15). Thus, the mucosal tolerance to E-selectin had a suppressive effect against the activation of vessels in the brain induced by protracted hypoperfusion.

Immunoassay: The sham group had a statistically significantly lower level of plasma TNF than the E-selectin and PBS groups by one-factor ANOVA (p<0.05). However, there were no significant differences on the level of plasma TNF between the E-selectin and PBS groups.

The results provided above illustrate the protective effect of mucosal tolerance to E-selectin against histological damage and functional impairments that develops during protracted cerebral hypoperfusion induced by the permanent occlusion of both common carotid arteries.

Because the severity of the damage in the optic nerve was attenuated in the E-selectin-treated group as compared to the PBS-treated group, the potential effect of differential visual acuity on behavioral tests such as the object recognition test should be considered. In this study, the discrimination ability preserved by E-selectin treatment was not correlated with the degree of protection from fiber loss in the optic nerve. One explanation for this discrepancy is that humans primarily base their choices on a memory of visual the properties of the sample object. In contrast, when rats explore an object, they sniff it, palpate it with vibrissae, and look at it. In the rodents, differential exploration of familiar objects and novel objects reflects to some extent their memory for olfactory and tactile properties of the sample object, although visual properties may also be remembered and contribute to discrimination.

There were apparent differences in the protective effects conferred by E-selectin tolerization on T maze left/right discrimination memory when tested at the 2, 6 and 10 week time points. In contrast to 10 weeks after surgery, at 2 and 6 weeks after surgery the E-selectin group did not retain their left/right discrimination memory. The impairment in the left/right discrimination memory at 2 and 6 weeks after surgery might have been caused by a decrease of cerebral blood flow that impaired function without permanent cortical white matter damage. Cerebral blood flow in this model remains reduced over a prolonged period and gradually recovers to control levels by 8 weeks (Otori T, Cerebrovasc. Dis. 6 (suppl): 71 (1996)).

In the present study, plasma TNF level was increased in the ischemic groups (PBS and E-selectin), as compared with the sham-operated group even 90 days after surgery. Since the expression of E-selectin is induced in response to TNF, these findings suggest that the endothelial activation and E-selectin induction could persist for a prolonged period under conditions of protracted hypoperfusion. Mucosal tolerance can be achieved through different mechanisms, including clonal anergy/deletion of antigen-reactive T cells, and active tolerance with induction of regulatory T cells (Faria A M, Adv Immunol. 73:153-264 (1999)). Clonal anergy/deletion can be induced by a single feeding of very high-dose antigen (Chen Y., Nature. 376(6536):177-180 (1995)) and the production of regulatory T cells occurs after repetitive administration of low-dose antigen (Groux H., Nature 389:737-742 (1997); Chen Y., Science 265:1237-1240 (1994)). Lymphocytes that are tolerized to an antigen and have become antigen-specific regulatory T-cells tend to migrate to the locale of the protein molecule to which they have been primed. In that location, they release immunomodulatory cytokines, such as TGF-β and IL-10 that counteract the effect of pro-inflammatory cytokines including TNF and suppress inflammation and immune responses after ischemia (Pang L., Stroke. 2001; 32:544-552 (2001), Hallenbeck J. M., Trends in Immunology 26:550-556 (2005)). Since local release of immunological and inflammatory mediators contributes to local vessel activation, local immunosuppression targeted to activating blood vessel segments could protect against local impairment of microcirculatory perfusion. In this study, the number of TNF immunopositive vessels and E-selectin immunopositive vessels were significantly decreased in the E-selectin treated group, compared to the PBS control group. But the plasma TNF level was not significantly decreased in the E-selectin group as compared with PBS group. These results indicate that local vessel activation and local TNF production was suppressed by the mucosal tolerance to E-selectin in a setting of undiminished systemic TNF production. TNF expressed by endothelium has proinflammatory and procoagulant effects on endothelium (Pober J S, Physiol Rev. 70:427-451 (1990), Hallenbeck J M. Nat. Med. 8:1363-1368 (2002)). E-selectin appears to function by suppressing local vessel activation and the surrounding immunological and inflammatory processes rather than by systemic immunosuppression.

Other mechanisms may also contribute to the protective effect in the present study. White matter injury involves glial cells, which are abundant in white matter (Goldberg M, Stroke 34:330-332 (2003)). In this model, microglial activation with expression of MHC class II antigens was detected preferentially in the white matter (Wakita H, Acta Neuropathol. (Berl) 87: 484-492 (1994); Farkas E., Acta Neuropathol (Berl). 108:57-64 (2004), Schmidt-Kastner R., Brain Res. 1052:28-39 (2005)), and pharmacological suppression of these activated microglia has resulted in an attenuation of the white matter lesions (Wakita H, Stroke 26:1415-1422 (1995); Wakita H, Brain Res. 792:105-113 (1998); Wakita H, Neuroreport 14:1461-1465 (1999), Wakita H., Brain Res. 992:53-59 (2003)).

Since TGF-β and IL-10 inhibit the activation of microglia (Suzumura A, J. Immunol. 151:2150-2158 (1993), Frei K, J. Immunol. 152:2720-2728 (1994)), mucosal tolerization to E-selectin suppresses the activated microglia through local production of these cytokines by the regulatory T cells. The number of MHC class II positive activated microglia/macrophages in the white matter showed a trend toward suppression in the E-selectin group as compared to the PBS group. Activated microglia may enhance a variety of inflammatory responses (Morioka T, J. Cereb. Blood Flow Metab. 1991; 11:966-973 (1991); Wakita H, Acta Neuropathol. (Berl) 87: 484-492 (1994); Gehrmann J, Brain Res. Rev. 20:269-287 (1995)). Microglia are the major source of pro-inflammatory cytokines including IL-1 and TNF, which may induce the expression of E-selectin in the ischemic cerebral vasculature. The suppression of the microglia may inhibit both local vessel activation and the expression of E-selectin. Activated microglia also release an array of cytotoxic substances that include other pro-inflammatory cytokines, prostanoids, proteases, reactive oxygen radicals and nitrogen intermediates. The protective effect may be mediated by suppressing the release of these cytotoxic substances as well. The net effect decreases inflammation and preserves vessel integrity.

In conclusion, the present study demonstrates the protective effect of mucosal tolerance to E-selectin against ischemic cerebrovascular white matter damage and memory impairment during protracted cerebral hypoperfusion. These results support a new therapeutic strategy that involves mucosal tolerization to E-selectin to protect against subcortical ischemic vascular cognitive impairment on a long-term basis.

### Example 5

### E-Selectin Administration Ameliorates Experimental Autoimmune Encephalitis (EAE)

This Example illustrates that intranasal administration of E-selectin can reduce the symptoms and severity of disease as well as delay the onset of experimental autoimmune encephalitis (EAE), which is an animal model for multiple sclerosis.

### Materials and Methods

Six week old female C57BL/6 mice were administered PBS, 0.1, 1.0, 5.0 or 10.0 μg of human recombinant E-selectin intranasally on days 0, 2, 4, 6, 8, 19, 21, 23, 25, and 27 of the experiment as shown by the schedule shown in FIG. 17. On day 29, active chronic EAE was induced in the mice by immunization with myelin oligodendroglial protein (MOG) peptide amino acids 35-55 in 2 mg/ml H37Ra mycobacterium in incomplete Freund's adjuvant, with pertusis toxin administration (200 ng, in PBS) I.P. Pertusis toxin was administered again on day 31. On days 38, 40, 42, 44, and 46, E-selectin was again administered to the animals.

Animals were weighed and scored daily according to the following relative scale of EAE symptoms: 0-no symptoms, 1-limp tail, 2-hind limb weakness, 2.5-moderate hind limb weakness, 3-single hind limb paralysis, 3.5-both hind limb paralysis, 4-forelimb weakness, 4.5-fore limb paralysis, 5-moribund. At days 45 or 60, animals were euthanized and tissues taken for analysis.

Table V provides results of the experiment.

TABLE V
(n = 5 per group)

| | | Median | Average | Average | Average |
|---|---|---|---|---|---|
| Agent | Dosage | Cumulative | Cumulative | day of | disease |
| administered | (μg) | disease score | disease score | onset | score |
| PBS | | 52.8 | 46.8 | 15.0 | 1.35 |
| Human E- | 0.1 | 38.0 | 39.5 | 16.3 | .91 |
| selectin | | | | | |
| Human E- | 1.0 | 52.0 | 51.4 | 16.0 | 1.48 |
| selectin | | | | | |
| Human E- | 5.0 | 16.0 | 31.0 | 17.8 | .88 |
| selectin | | | | | |
| Human E- | 10.0 | 43.8 | 43.6 | 16.2 | 1.25 |
| selectin | | | | | |

TABLE VI
(n = 7 per group)

| | | | Average | | |
|---|---|---|---|---|---|
| | | Median | Cumulative | Average | Average |
| Agent | Dosage | Cumulative | disease | day of | disease |
| administered | (μg) | disease score | score | onset | score |
| PBS | | 81.5 | 70.3 | 13.4 | 2.56 |
| Human E- | 0.1 | 39.5 | 49.9 | 19.0 | 1.20 |
| selectin | | | | | |
| Human E- | 1.0 | 39.75 | 55.0 | 19.9 | 1.51 |
| selectin | | | | | |
| Human E- | 5.0 | 34.0 | 37.1 | 17.5 | 1.22 |
| selectin | | | | | |
| Human E- | 10.0 | 47.0 | 55.8 | 18.7 | 1.44 |
| selectin | | | | | |

### Results

E-selectin administration reduced the cumulative disease score at all doses tested relative to mice receiving PBS/sham agent (Tables V-VI). The cumulative disease score is the summation of daily scores over the course of the experiment. The average severity of the disease in terms of average daily score was also reduced by E-selectin administration relative to controls. E-selectin administration also tended to delay the onset of disease.

### Example 6

### Immune Response to E-Selectin

Splenic populations were isolated from the spleens of mice described in Example 5 (n=7) and tested for T cell proliferation/thymidine incorporation in response to various concentrations of human recombinant E-selectin. The results are reported in Tables VII and VIII as the SI (stimulation index) relative to negative controls for T proliferation/thymidine incorporation in response to human recombinant E-selectin (Novavax), human recombinant E-selectin (R&D Systems) and mouse recombinant E-selectin (R&D Systems).

### Results

T cell proliferative responses to human E-selectin were clearly present in animals which had previously been administered human E-selectin (Table VII). Sham or PBS-administered animals showed negligible proliferative responses to human E-selectin. Responses only seemed to occur at relatively high concentrations of human recombinant E-selectin. There was no apparent correlation between the dose and the observed proliferative response to E-selectin.

TABLE VII
T cell Proliferation/Thymidine Incorporation Stimulation Index Relative to Negative controls

| | Administration regimen | | | | | | | | | |
|---|---|---|---|---|---|---|---|---|---|---|
| | PBS | | E sel 0.1 μg | | E sel 1.0 μg | | E sel 5.0 μg | | E sel 10.0 μg | |
| μg/mL | mouse a | mouse b | mouse a | mouse b | mouse a | mouse b | mouse a | mouse b | mouse a | mouse b |
| 100 | 2.6 | 1.5 | 13.6 | 7.3 | 11.9 | 7.3 | 6.6 | 15.0 | 7.0 | 3.2 |
| 20 | 2.0 | 1.3 | 6.0 | 5.0 | 7.0 | 5.0 | 3.8 | 4.8 | 5.3 | 3.0 |
| 4 | 1.4 | 1.1 | 5.2 | 3.4 | 5.9 | 3.4 | 2.6 | 3.0 | 2.1 | 1.6 |
| 0.8 | 1.3 | 1.3 | 3.4 | 2.4 | 3.9 | 2.7 | 2.6 | 2.0 | 2.3 | 1.4 |
| 0.16 | 1.3 | 0.9 | 2.1 | 1.9 | 2.8 | 2.2 | 1.7 | 1.7 | 1.5 | 1.3 |
| 0.032 | 1.3 | 1.1 | 2.0 | 2.0 | 1.6 | 1.5 | 1.6 | 1.5 | 1.6 | 1.2 |
| 0.0064 | 1.2 | 1.2 | 1.6 | 1.3 | 2.6 | 1.5 | 1.5 | 1.6 | 1.1 | 1.2 |
| 0 | 1.0 | 1.0 | 1.2 | 0.9 | 0.8 | 1.1 | 1.0 | 1.1 | 1.0 | 1.0 |

TABLE VIII

| | | Administration Regimen | | | |
|---|---|---|---|---|---|
| | Dose | | | E sel | E sel |
| Antigen | (μg/ml) | PBS | PBS | 0.1 μg | 1.0 μg |
| Control media | — | .9 | 1.0 | 1.0 | .9 |
| Anti-CD3 | 1 | 11.6 | 10.8 | 19.1 | 59.1 |
| Human E-selectin (Novavax) | 100 | Nd | 10.8 | 12.6 | Nd |
| | 20 | Nd | 2.6 | 8.3 | Nd |
| | 4 | .9 | .9 | 8.2 | 6.4 |
| Human E-selectin (R&D | 20 | Nd | 1.3 | 4.7 | Nd |
| System) | 4 | .8 | 1.0 | 4.8 | 1.8 |
| Murine E-selectin (R&D | 20 | Nd | .9 | 2.1 | Nd |
| System) | 4 | .9 | .8 | 1.7 | 1.9 |

Nd = test not done.

Values represent the SI or stimulation index which is the fold increase of treatment groups compared to untreated or control media-treated animals. Animals administered PBS/sham as well as those administered E-selectin showed high proliferation/fold increases in response to anti-CD3 stimulation (Table VIII). While PBS/sham administered animals showed negligible responses to any of the E-selectins, E-selectin administered animals showed significant responses to both forms of the human E-selectin. Moreover, E-selectin administered animals showed proliferative responses to murine E-selectin, albeit at lower levels than to human E-selectin. This data suggests that an immune response specific to E-selectin is being generated/induced within these mice by the administration process.

### Example 7

### IL10 Production after E-selectin Administration

Splenic populations from animals administered PBS or E-selectin were tested for their ability to produce IL-10 in response to E-selectin by a sandwich ELISA. Values represent the amount of IL-10 cytokine in pg/ml measured as produced in 72 hours post antigenic stimulation (Table IX).

### Results

In one of the two mice which received E-selectin, IL-10 was produced in significantly greater amounts by splenic cells in after further administration of E-selectin. No group showed spontaneous IL-10 production in control media, nor in response to global anti-CD3 stimulation.

TABLE IX
Production of IL-10

Administration regimen

| | | | 0.1 ug E- | 1.0 ug E- |
|---|---|---|---|---|
| Stimulus | PBS | PBS | selectin | selectin |
| Control media | ND | ND | ND | ND |
| Anti-CD3 | ND | ND | ND | ND |
| Human E-selectin | ND | ND | 105 | ND |
| (100 ug/ml) | | | | |

ND = none detected/lower than the detection limit of the assay ≦31.2 pg/ml.

These results suggested that in E-selectin administered mice, IL-10 is specifically produced in response to subsequent challenge with E-selectin. This E-selectin specific production of the regulatory cytokine IL-10 may contribute to the immunosuppressive/regulatory effects observed and is one potential mechanism of action to reduce the severity of the disease or suppress the development of the disease.

### Example 8

### Homologous (Murine) E-Selectin Administration Ameliorates Experimental Autoimmune Encephalitis (EAE)

This Example illustrates that intranasal administration of homologus murine E-selectin can reduce the symptoms and severity of disease as well as delay the onset of EAE.

Materials and Methods: intranasal administration regimen and induction of EAE are similar to those in Example 5.

### Results

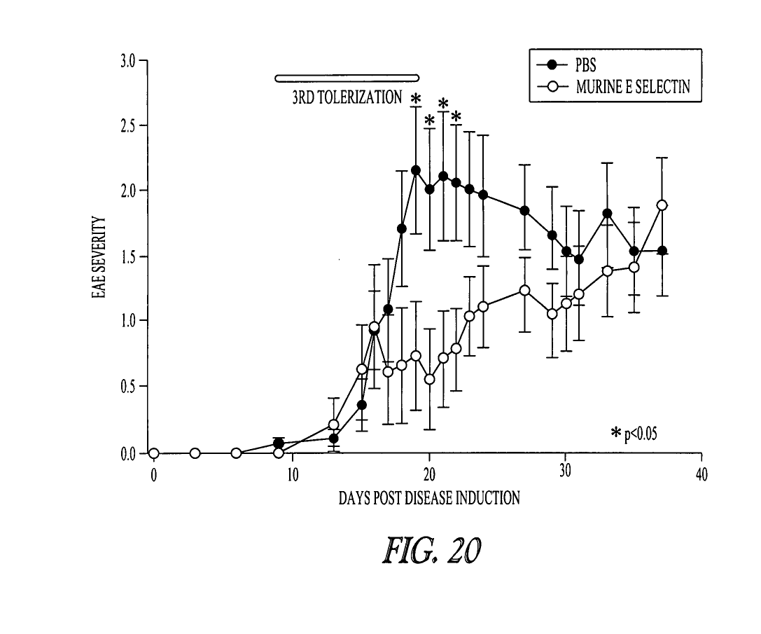

Administration of murine E selectin led to a reduction in the severity of EAE during the initial phase of the disease (FIG. 20). This reduction in disease severity was observed from onset through the third tolerization regimen and was statistically significant days 19 through 23 post immunization (p<0.05 by t-test compared to PBS-treated controls, n=10 per group). By 40 days post immunization, the severity of the disease was not statistically different from animals receiving PBS, suggesting that intranasal tolerization delayed the onset of disease. Cumulative disease scores for E selectin tolerized animals were lower than PBS tolerized animals (27.8+/−18 versus 17.2+/−17.1) but were not statistically significant over this time course, most likely due to the gradual onset of disease once intranasal administration had ceased. This suggests that murine E selectin delivered intranasally in a homologous system modulates the development of CNS autoimmunity.

### Example 9

### E-selectin Sequences Employed

For early experiments, a human E-selectin polypeptide with SEQ ID NO:30 was made by recombinant procedures using a pNVAX1002 expression vector. This SEQ ID NO:30 sequence is shown below.

| | |
|---|---|
| 1 | MGWSWIFLFL LSGTASVHSW SYNTSTEAMT YDEASAYCQQ |
| 41 | RYTHLVAIQN KEEIEYLNSI LSYSPSYYWI GIRKVNNVWV |
| 81 | WVGTQKPLTE EAKNWAPGEP NNRQKDEDCV EIYIKREKDV |
| 121 | GMWNDERCSK KKLALCYTAA CTNTSCSGHG ECVETINNYT |
| 161 | CKCDPGFSGL KCEQIVNCTA LESPEHGSLV CSHPLGNFSY |
| 201 | NSSCSISCDR GYLPSSMETM QCMSSGEWSA PIPACNVVEC |
| 241 | DAVTNPANGF VECFQNPGSF PWNTTCTFDC EEGFELMGAQ |
| 281 | SLQCTSSGNW DNEKPTCKAV TGGASTRAAE QKLISEEDLN |
| 321 | GTRSGHHHHH H |

This SEQ ID NO:30 sequence has a signal sequence (MGWSWIFLFL LSGTASVHS (SEQ ID NO:27)), which is cleaved during recombinant production and is not present in the purified product. The SEQ ID NO:30 E-selectin sequence also has a histidine tag sequence (GGASTRAAEQKLI SEEDLNGTRSGHHHHHH (SEQ ID NO:29)), which can facilitate isolation and detection of the E-selectin. Upon removal of the MGWSWIFLFL LSGTASVHS (SEQ ID NO:27) signal sequence a polypeptide with the following E-selectin polypeptide with SEQ ID NO:31 is generated.

| | |
|---|---|
| 1 | W SYNTSTEAMT YDEASAYCQQ |
| 41 | RYTHLVAIQN KEEIEYLNSI LSYSPSYYWI GIRKVNNVWV |
| 81 | WVGTQKPLTE EAKNWAPGEP NNRQKDEDCV EIYIKREKDV |
| 121 | GMWNDERCSK KKLALCYTAA CTNTSCSGHG ECVETINNYT |
| 161 | CKCDPGFSGL KCEQIVNCTA LESPEHGSLV CSHPLGNFSY |
| 201 | NSSCSISCDR GYLPSSMETM QCMSSGEWSA PIPACNVVEC |
| 241 | DAVTNPANGF VECFQNPGSF PWNTTCTFDC EEGFELMGAQ |
| 281 | SLQCTSSGNW DNEKPTCKAV TGGASTRAAE QKLISEEDLN |
| 321 | GTRSGHHHHH H |

For somewhat later experiments, a human E-selectin polypeptide with SEQ ID NO:32 was made by recombinant procedures using a pNVAX1037 expression vector. This SEQ ID NO:32 sequence is shown below.

| | |
|---|---|
| 1 | MGWSWIFLFL LSGTASVHSW SYNTSTEAMT YDEASAYCQQ |
| 41 | RYTHLVAIQN KEEIEYLNSI LSYSPSYYWI GIRKVNNVWV |
| 81 | WVGTQKPLTE EAKNWAPGEP NNRQKDEDCV EIYIKREKDV |
| 121 | GMWNDERCSK KKLALCYTAA CTNTSCSGHG ECVETINNYT |
| 161 | CKCDPGFSGL KCEQIVNCTA LESPEHGSLV CSHPLGNFSY |
| 201 | NSSCSISCDR GYLPSSMETM QCMSSGEWSA PIPACNVVEC |
| 241 | DAVTNPANGF VECFQNPGSF PWNTTCTFDC EEGFELMGAQ |
| 281 | SLQCTSSGNW DNEKPTCKAV T |

This SEQ ID NO:32 sequence has a signal sequence (MGWSWIFLFL LSGTASVHS (SEQ ID NO:27)) but no histidine tag sequence. As indicated above the SEQ ID NO:27 signal sequence is cleaved during recombinant production and is not present in the purified product. Upon removal of the SEQ ID NO:27 signal sequence, this E-selectin polypeptide has SEQ ID NO:8.

For more recent experiments, a mouse E-selectin polypeptide with SEQ ID NO:33 was made by recombinant procedures using a pNVAX1076 expression vector. This SEQ ID NO:33 sequence is shown below.

| | |
|---|---|
| 1 | MPLYKLLNVL WLVAVSNAIW YYNASSELMT YDEASAYCQR |
| 41 | DYTHLVAIQN KEEINYLNSN LKHSPSYYWI GIRKVNNVWI |
| 81 | WVGTGKPLTE EAQNWAPGEP NNKQRNEDCV EIYIQRTKDS |
| 121 | GMWNDERCNK KKLALCYTAS CTNASCSGHG ECIETINSYT |
| 161 | CKCHPGFLGP NCEQAVTCKP QEHPDYGSLN CSHPFGPFSY |
| 201 | NSSCSFGCKR GYLPSSMETT VRCTSSGEWS APAPACHVVE |
| 241 | CEALTHPAHG IRKCSSNPGS YPWNTTCTFD CVEGYRRVGA |
| 281 | QNLQCTSSGI WDNETPSCKA VT |

This SEQ ID NO:33 sequence has an N-terminal signal sequence (MPLYKLLNVLWLVAVSNAI (SEQ ID NO:28)), which is cleaved and lost during recombinant production of the E-selectin product. Upon removal of the SEQ ID NO:28 signal sequence, this E-selectin polypeptide has SEQ ID NO:19.

Recent experiments have also employed a mouse E-selectin polypeptide with SEQ ID NO:18 was made by recombinant procedures using a pNVAX1189 expression vector. This SEQ ID NO:18 sequence is shown below.

| | |
|---|---|
| 1 | MGWSWIFLFL LSGTASVHSW YYNASSELMT YDEASAYCQR |
| 41 | DYTHLVAIQN KEEINYLNSN LKHSPSYYWI GIRKVNNVWI |
| 81 | WVGTGKPLTE EAQNWAPGEP NNKQRNEDCV EIYIQRTKDS |
| 121 | GMWNDERCNK KKLALCYTAS CTNASCSGHG ECIETINSYT |
| 161 | CKCHPGFLGP NCEQAVTCKP QEHPDYGSLN CSHPFGPFSY |
| 201 | NSSCSFGCKR GYLPSSMETT VRCTSSGEWS APAPACHVVE |
| 241 | CEALTHPAHG IRKCSSNPGS YPWNTTCTFD CVEGYRRVGA |
| 281 | QNLQCTSSGI WDNETPSCKA VT |

This SEQ ID NO:18 sequence has a signal sequence (MGWSWIFLFL LSGTASVHS (SEQ ID NO:27)), which is cleaved during recombinant production and is not present in the purified product. Upon removal of the SEQ ID NO:27 signal sequence, this E-selectin polypeptide has SEQ ID NO:19.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method for inhibiting the progression or severity of nerve damage associated with inflammation in a mammal comprising mucosal administration to the mammal of an effective amount of E-selectin polypeptide to thereby inhibit the progression or severity of nerve damage in the mammal.

2. The method of claim 1, wherein the nerve damage is associated with a T cell mediated autoimmune disorder or multiple sclerosis.

3. (canceled)

4. The method of claim 1, wherein the E-selectin comprises SEQ ID NO:5-8, 18, 19 or a combination thereof.

5. The method of claim 1, wherein the mucosal administration of E-selectin comprises intranasal, oral, enteral, vaginal, rectal, or respiratory administration.

6. (canceled)

7. The method of claim 1 of, which further comprises a first series of administrations of E-selectin over a period of about two weeks.

8. The method of claim 7, wherein the first series of administrations comprises about three to about seven administrations of E-selectin over the period of about two weeks.

9. The method of claim 7, which further comprises at least one booster series of administrations of E-selectin after at least two weeks from the first series of administrations, wherein each booster series of administrations comprise about three to about seven administrations of E-selectin over the period of two weeks.

10. (canceled)

11. The method of claim 1, wherein the effective amount of E-selection is an amount effective for inducing expression of transforming growth factor beta (TGFβ), interleukin-4 (IL-4) and/or interleukin-10 (IL-10).

12. The method of claim 1, wherein the effective amount of E-selectin is about 0.005 mg to about 500 mg.

13. (canceled)

14. The method of claim 1, wherein the administration is dry or atomized aqueous aerosol administration.

15. (canceled)

16. (canceled)

17. A method for treating or inhibiting localized vascular inflammation in a mammal comprising mucosal administration to the mammal of an effective amount of E-selectin polypeptide comprising SEQ ID NO:5-8, 18, 19 or a combination thereof to thereby treat or inhibit localized vascular inflammation.

18. The method of claim 17, wherein blood flow to the mammal's brain is or may become reduced or the mammal suffers or may suffer from stroke or vascular dementia.

19. (canceled)

20. The method of claim 17, wherein the mucosal administration of E-selectin comprises intranasal, oral, enteral, vaginal, rectal, or respiratory administration.

21. (canceled)

22. The method of claim 17, which further comprises a first series of administrations of E-selectin over a period of about two weeks.

23. The method of claim 22, wherein the first series of administrations comprises about three to about seven administrations of E-selectin over the period of about two weeks.

24. The method of claim 22, which further comprises at least one booster series of administrations of E-selectin after at least two weeks from the first series of administrations, wherein each booster series of administrations comprise about three to about seven administrations of E-selectin over the period of two weeks.

25. (canceled)

26. The method of claim 17, wherein the administration is dry or atomized aqueous aerosol administration.

27. (canceled)

28. (canceled)

29. The method of claim 17, wherein the effective amount of E-selectin is sufficient to promote bystander suppression-effect tolerance to E-selectin in a mammal or is an amount effective for inducing expression of transforming growth factor beta (TGFβ), interleukin-4 (IL-4) and/or interleukin-10 (IL-10).

30. (canceled)

31. The method of claim 1, wherein the effective amount of E-selectin is about 0.005 microgram to about 500 milligram.

32. (canceled)

33. A pharmaceutical composition comprising a pharmaceutically acceptable carrier and a therapeutically effective amount of an E-selectin comprising SEQ ID NO:8, 19 or a combination thereof.

34. The pharmaceutical composition of claim 33, wherein the composition is formulated for mucosal administration.

35. The pharmaceutical composition of claim 34, wherein the mucosal administration is intranasal, oral, enteral, vaginal, rectal, or respiratory.

36. (canceled)

37. The pharmaceutical composition of claim 33, wherein the composition is formulated as a dry or atomized aqueous aerosol.

38. (canceled)

39. (canceled)

40. The pharmaceutical composition of claim 33, wherein a therapeutically effective amount of E-selectin is sufficient to induce tolerance or promote bystander suppression-effect tolerance to E-selectin in a mammal.

41. (canceled)

42. The pharmaceutical composition of claim 33, wherein the effective amount of E-selection is an amount effective for inducing expression of transforming growth factor beta (TGFβ), interleukin-4 (IL-4) and/or interleukin-10 (IL-10) in a mammal.

43. The pharmaceutical composition of claim 33, wherein a therapeutically effective amount of E-selectin is about 0.005 μg to about 500 mg.

44. The pharmaceutical composition of claim 33, wherein a therapeutically effective amount of E-selectin is about 0.05 μg to about 50 mg.

45. (canceled)

46. (canceled)

47. (canceled)

48. (canceled)

49. (canceled)

50. (canceled)

51. (canceled)

52. (canceled)

53. (canceled)

54. The method of claim 17, wherein the effective amount of E-selectin is about 0.005 microgram to about 500 milligram.


SEQUENCE LISTING (33 sequences)

SEQ ID NO: 1 - protein, 610 aa, Homo sapiens
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 2 - protein, 589 aa, Homo sapiens
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 3 - protein, 535 aa, Homo sapiens
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIP

SEQ ID NO: 4 - protein, 138 aa, Homo sapiens
PKGLVRCAHSPIGEFTYKSSCAFSCEEGFELYGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPF

SEQ ID NO: 5 - protein, 306 aa, Homo sapiens
MPLYKLLNVLWLVAVSNAIPGSWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTRS

SEQ ID NO: 6 - protein, 304 aa, Homo sapiens
MPLYKLLNVLWLVAVSNAIPGSWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVT

SEQ ID NO: 7 - protein, 284 aa, Homo sapiens
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTRS

SEQ ID NO: 8 - protein, 282 aa, Homo sapiens
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVT

SEQ ID NO: 9 - protein, 485 aa, Bovine
MIVSQYLSALTFVLLLFKESRTWSYHASTEMMTFEEARDYCQKTYTALVAIQNQEEIEYLNSTFSYSPSYYWIGIRKINGTWTWIGTNKSLTKEATNWAPGEPNNKQSDEDCVEIYIKREKDSGKWNDEKCTKQKLALCYKAACNPTPCGSHGECVETINNYTCQCHPGFKGLKCEQVVTCPAQKHPEHGHLVCNPLGKFTYNSSCSISCAEGYLPSSTEATRCMSSGEWSTPLPKCNVVKCDALSNLDNGVVNCSPNHGSLPWNTTCTFECQEGYKLTGPQHLQCTSSGIWDNKQPTCKAVSCAAISHPQNGTVNCSHSVVGDFAFKSSCHFTCAEGFTLQGPTQVECTAQGQWTQRVPVCEVVRCSRLDVSGKLNMNCSGEPVLGTECTFACPERWTLNGSVVLTCGATGHWSGMLPTCEAPTVSQTPLAVGLSTAGVSLVTIPSFLFWLLKRLQKKAKKFSPASSCSSLKSNGCYSTPSKLI

SEQ ID NO: 10 - protein, 463 aa, Bovine
WSYHASTEMMTFEEARDYCQKTYTALVAIQNQEEIEYLNSTFSYSPSYYWIGIRKINGTWTWIGTNKSLTKEATNWAPGEPNNKQSDEDCVEIYIKREKDSGKWNDEKCTKQKLALCYKAACNPTPCGSHGECVETINNYTCQCHPGFKGLKCEQVVTCPAQKHPEHGHLVCNPLGKFTYNSSCSISCAEGYLPSSTEATRCMSSGEWSTPLPKCNVVKCDALSNLDNGVVNCSPNHGSLPWNTTCTFECQEGYKLTGPQHLQCTSSGIWDNKQPTCKAVSCAAISHPQNGTVNCSHSVVGDFAFKSSCHFTCAEGFTLQGPTQVECTAQGQWTQRVPVCEVVRCSRLDVSGKLNMNCSGEPVLGTECTFACPERWTLNGSVVLTCGATGHWSGMLPTCEAPTVSQTPLAVGLSTAGVSLVTIPSFLFWLLKRLQKKAKKFSPASSCSSLKSNGCYSTPSKLI

SEQ ID NO: 11 - protein, 408 aa, Bovine
WSYHASTEMMTFEEARDYCQKTYTALVAIQNQEEIEYLNSTFSYSPSYYWIGIRKINGTWTWIGTNKSLTKEATNWAPGEPNNKQSDEDCVEIYIKREKDSGKWNDEKCTKQKLALCYKAACNPTPCGSHGECVETINNYTCQCHPGFKGLKCEQVVTCPAQKHPEHGHLVCNPLGKFTYNSSCSISCAEGYLPSSTEATRCMSSGEWSTPLPKCNVVKCDALSNLDNGVVNCSPNHGSLPWNTTCTFECQEGYKLTGPQHLQCTSSGIWDNKQPTCKAVSCAAISHPQNGTVNCSHSVVGDFAFKSSCHFTCAEGFTLQGPTQVECTAQGQWTQRVPVCEVVRCSRLDVSGKLNMNCSGEPVLGTECTFACPERWTLNGSVVLTCGATGHWSGMLPTCEAPTVSQTP

SEQ ID NO: 12 - protein, 549 aa, Rattus norvegicus
MNASCFLSALTFVLLIGKSIAWYYNASSELMTYDEASAYCQRDYTHLVAIQNKEEINYLNSTLRYSPSYYWIGIRKVNNVWIWVGTQKPLTEEAKNWAPGEPNNKQRNEDCVEIYIQRPKDSGMWNDERCDKKKLALCYTASCTNTSCSGHGECVETINSYTCKCHPGFLGPKCDQVVTCQEQEYPDHGSLNCTHPFGLFSYNSSCSFSCERGYVPSSMETTVRCTSSGEWSAPAPACHVVECKALTQPAHGVRKCSSNPGSYPWNTTCTFDCEEGYRRVGAQNLQCTSSGVWDNEKPSCKAVTCDAIPRPQNGSVSCSNSTAGALAFKSSCNFTCEHSFTLQGPAQVECSAQGQWTPQIPVCKASQCEALSAPQRGHMKCLPSASAPFQSGSSCKFSCDEGFELKGSRRLQCGPRGEWDSEKPTCAGVQCSSLDLPGKMNMSCSGPAVFGTVCEFTCPEGWTLNGSSILTCGATGRWSAMLPTCEAPANPPRPLVVALSVAATSLLTLSSLIYVLKRFFWKKAKKFVPASSCQSLQSFENYQGPSYII

SEQ ID NO: 13 - protein, 528 aa, Rattus norvegicus
WYYNASSELMTYDEASAYCQRDYTHLVAIQNKEEINYLNSTLRYSPSYYWIGIRKVNNVWIWVGTQKPLTEEAKNWAPGEPNNKQRNEDCVEIYIQRPKDSGMWNDERCDKKKLALCYTASCTNTSCSGHGECVETINSYTCKCHPGFLGPKCDQVVTCQEQEYPDHGSLNCTHPFGLFSYNSSCSFSCERGYVPSSMETTVRCTSSGEWSAPAPACHVVECKALTQPAHGVRKCSSNPGSYPWNTTCTFDCEEGYRRVGAQNLQCTSSGVWDNEKPSCKAVTCDAIPRPQNGSVSCSNSTAGALAFKSSCNFTCEHSFTLQGPAQVECSAQGQWTPQIPVCKASQCEALSAPQRGHMKCLPSASAPFQSGSSCKFSCDEGFELKGSRRLQCGPRGEWDSEKPTCAGVQCSSLDLPGKMNMSCSGPAVFGTVCEFTCPEGWTLNGSSILTCGATGRWSAMLPTCEAPANPPRPLVVALSVAATSLLTLSSLIYVLKRFFWKKAKKFVPASSCQSLQSFENYQGPSYII

SEQ ID NO: 14 - protein, 474 aa, Rattus norvegicus
AWYYNASSELMTYDEASAYCQRDYTHLVAIQNKEEINYLNSTLRYSPSYYWIGIRKVNNVWIWVGTQKPLTEEAKNWAPGEPNNKQRNEDCVEIYIQRPKDSGMWNDERCDKKKLALCYTASCTNTSCSGHGECVETINSYTCKCHPGFLGPKCDQVVTCQEQEYPDHGSLNCTHPFGLFSYNSSCSFSCERGYVPSSMETTVRCTSSGEWSAPAPACHVVECKALTQPAHGVRKCSSNPGSYPWNTTCTFDCEEGYRRVGAQNLQCTSSGVWDNEKPSCKAVTCDAIPRPQNGSVSCSNSTAGALAFKSSCNFTCEHSFTLQGPAQVECSAQGQWTPQIPVCKASQCEALSAPQRGHMKCLPSASAPFQSGSSCKFSCDEGFELKGSRRLQCGPRGEWDSEKPTCAGVQCSSLDLPGKMNMSCSGPAVFGTVCEFTCPEGWTLNGSSILTCGATGRWSAMLPTCEAPANPPRP

SEQ ID NO: 15 - protein, 612 aa, Mus musculus
MNASRFLSALVFVLLAGESTAWYYNASSELMTYDEASAYCQRDYTHLVAIQNKEEINYLNSNLKHSPSYYWIGIRKVNNVWIWVGTGKPLTEEAQNWAPGEPNNKQRNEDCVEIYIQRTKDSGMWNDERCNKKKLALCYTASCTNASCSGHGECIETINSYTCKCHPGFLGPNCEQAVTCKPQEHPDYGSLNCSHPFGPFSYNSSCSFGCKRGYLPSSMETTVRCTSSGEWSAPAPACHVVECEALTHPAHGIRKCSSNPGSYPWNTTCTFDCVEGYRRVGAQNLQCTSSGIWDNETPSCKAVTCDAIPQPQNGFVSCSHSTAGELAFKSSCNFTCEQSFTLQGPAQVECSAQGQWTPQIPVCKAVQCEALSAPQQGNMKCLPSASGPFQNGSSCEFSCEEGFELKGSRRLQCGPRGEWDSKKPTCSAVKCDDVPRPQNGVMECAHATTGEFTYKSSCAFQCNEGFSLHGSAQLECTSQGKWTQEVPSCQVVQCPSLDVPGKMNMSCSGTAVFGTVCEFTCPDDWTLNGSAVLTCGATGRWSGMPPTCEAPVSPTRPLVVALSAAGTSLLTSSSLLYLLMRYFRKKAKKFVPASSCQSLQSFENYHVPSYNV

SEQ ID NO: 16 - protein, 591 aa, Mus musculus
WYYNASSELMTYDEASAYCQRDYTHLVAIQNKEEINYLNSNLKHSPSYYWIGIRKVNNVWIWVGTGKPLTEEAQNWAPGEPNNKQRNEDCVEIYIQRTKDSGMWNDERCNKKKLALCYTASCTNASCSGHGECIETINSYTCKCHPGFLGPNCEQAVTCKPQEHPDYGSLNCSHPFGPFSYNSSCSFGCKRGYLPSSMETTVRCTSSGEWSAPAPACHVVECEALTHPAHGIRKCSSNPGSYPWNTTCTFDCVEGYRRVGAQNLQCTSSGIWDNETPSCKAVTCDAIPQPQNGFVSCSHSTAGELAFKSSCNFTCEQSFTLQGPAQVECSAQGQWTPQIPVCKAVQCEALSAPQQGNMKCLPSASGPFQNGSSCEFSCEEGFELKGSRRLQCGPRGEWDSKKPTCSAVKCDDVPRPQNGVMECAHATTGEFTYKSSCAFQCNEGFSLHGSAQLECTSQGKWTQEVPSCQVVQCPSLDVPGKMNMSCSGTAVFGTVCEFTCPDDWTLNGSAVLTCGATGRWSGMPPTCEAPVSPTRPLVVALSAAGTSLLTSSSLLYLLMRYFRKKAKKFVPASSCQSLQSFENYHVPSYNV

SEQ ID NO: 17 - protein, 536 aa, Mus musculus
WYYNASSELMTYDEASAYCQRDYTHLVAIQNKEEINYLNSNLKHSPSYYWIGIRKVNNVWIWVGTGKPLTEEAQNWAPGEPNNKQRNEDCVEIYIQRTKDSGMWNDERCNKKKLALCYTASCTNASCSGHGECIETINSYTCKCHPGFLGPNCEQAVTCKPQEHPDYGSLNCSHPFGPFSYNSSCSFGCKRGYLPSSMETTVRCTSSGEWSAPAPACHVVECEALTHPAHGIRKCSSNPGSYPWNTTCTFDCVEGYRRVGAQNLQCTSSGIWDNETPSCKAVTCDAIPQPQNGFVSCSHSTAGELAFKSSCNFTCEQSFTLQGPAQVECSAQGQWTPQIPVCKAVQCEALSAPQQGNMKCLPSASGPFQNGSSCEFSCEEGFELKGSRRLQCGPRGEWDSKKPTCSAVKCDDVPRPQNGVMECAHATTGEFTYKSSCAFQCNEGFSLHGSAQLECTSQGKWTQEVPSCQVVQCPSLDVPGKMNMSCSGTAVFGTVCEFTCPDDWTLNGSAVLTCGATGRWSGMPPTCEAPVSPTRP

SEQ ID NO: 18 - protein, 302 aa, Mus musculus
MGWSWIFLFLLSGTASVHSWYYNASSELMTYDEASAYCQRDYTHLVAIQNKEEINYLNSNLKHSPSYYWIGIRKVNNVWIWVGTGKPLTEEAQNWAPGEPNNKQRNEDCVEIYIQRTKDSGMWNDERCNKKKLALCYTASCTNASCSGHGECIETINSYTCKCHPGFLGPNCEQAVTCKPQEHPDYGSLNCSHPFGPFSYNSSCSFGCKRGYLPSSMETTVRCTSSGEWSAPAPACHVVECEALTHPAHGIRKCSSNPGSYPWNTTCTFDCVEGYRRVGAQNLQCTSSGIWDNETPSCKAVT

SEQ ID NO: 19 - protein, 283 aa, Mus musculus
WYYNASSELMTYDEASAYCQRDYTHLVAIQNKEEINYLNSNLKHSPSYYWIGIRKVNNVWIWVGTGKPLTEEAQNWAPGEPNNKQRNEDCVEIYIQRTKDSGMWNDERCNKKKLALCYTASCTNASCSGHGECIETINSYTCKCHPGFLGPNCEQAVTCKPQEHPDYGSLNCSHPFGPFSYNSSCSFGCKRGYLPSSMETTVRCTSSGEWSAPAPACHVVECEALTHPAHGIRKCSSNPGSYPWNTTCTFDCVEGYRRVGAQNLQCTSSGIWDNETPSCKAVT

SEQ ID NO: 20 - DNA, 909 bp, Artificial Sequence
  A synthetic codon-optimized nucleic acid encoding the mouse E-selectin polypeptide with SEQ ID NO18
atgggttggtcctggatcttcctgtttctcttgtctggcaccgctagcgtgcactcatggtactataacgcctcgagtgagcttatgacttacgacgaagcgtccgcatactgccagcgtgattatacacatctggtcgctattcaaaataaggaggaaatcaactacctcaattctaacttgaaacacagcccctcatactattggattggaatccgcaaggttaacaatgtatggatctgggtgggtacgggcaaacctcttaccgaggaagcccagaactgggcgccaggagagccgaacaataagcaaaggaacgaagattgtgtcgagatttacatccagagaactaaggattcgggtatgtggaacgacgaacgatgcaataaaaagaagctggcactctgttacacagctagttgcacgaacgcctcctgttctggccatggagagtgcattgagaccatcaacagctatacttgcaaatgtcaccccggtttcttgggccctaattgcgaacaagctgttacatgtaagccacaggagcacccggattacggatcactgaactgctcccatcccttcggtcctttttcgtacaatagttcttgcagcttcggctgtaaacgtggatatcttccatcatccatggaaaccacggtacgctgcacttcgagtggtgagtggtctgcgccggcccccgcatgtcacgtggtcgaatgcgaggctctcacccatcctgcccacggcatcaggaagtgcagctccaacccaggatcatacccctggaacacaacttgtaccttcgactgcgttgaaggttacagacgtgtgggcgcgcaaaatttgcagtgtacgtcgtctggaatttgggacaacgagacacctagttgcaaggctgtcacttaa

SEQ ID NO: 21 - protein, 610 aa, Homo sapiens; identical to SEQ ID NO: 1

SEQ ID NO: 22 - protein, 610 aa, Homo sapiens
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPCFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELYGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 23 - protein, 306 aa, Artificial Sequence; identical to SEQ ID NO: 5
  A synthetic recombinant E-Selectin

SEQ ID NO: 24 - protein, 331 aa, Artificial Sequence
  A synthetic recombinant E-Selectin protein with c-myc
MGWSWIFLFLLSGTASVHSWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTGGASTRAAEQKLISEEDLNGTRSGHHHHHH

SEQ ID NO: 25 - protein, 301 aa, Homo sapiens
MGWSWIFLFLLSGTASVHSWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVT

SEQ ID NO: 26 - protein, 302 aa, Mus musculus; identical to SEQ ID NO: 18

SEQ ID NO: 27 - protein, 19 aa, Artificial Sequence
  A synthetic signal sequence
MGWSWIFLFLLSGTASVHS

SEQ ID NO: 28 - protein, 19 aa, Artificial Sequence
  A synthetic signal sequence
MPLYKLLNVLWLVAVSNAI

SEQ ID NO: 29 - protein, 30 aa, Artificial Sequence
  A synthetic C-terminal tag sequence
GGASTRAAEQKLISEEDLNGTRSGHHHHHH

SEQ ID NO: 30 - protein, 331 aa, Artificial Sequence; identical to SEQ ID NO: 24
  A synthetic human E-selectin polypeptide

SEQ ID NO: 31 - protein, 312 aa, Artificial Sequence
  A synthetic human E-selectin polypeptide
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTGGASTRAAEQKLISEEDLNGTRSGHHHHHH

SEQ ID NO: 32 - protein, 301 aa, Artificial Sequence; identical to SEQ ID NO: 25
  A synthetic human E-selectin polypeptide

SEQ ID NO: 33 - protein, 302 aa, Artificial Sequence
  A synthetic mouse E-selectin polypeptide
MPLYKLLNVLWLVAVSNAIWYYNASSELMTYDEASAYCQRDYTHLVAIQNKEEINYLNSNLKHSPSYYWIGIRKVNNVWIWVGTGKPLTEEAQNWAPGEPNNKQRNEDCVEIYIQRTKDSGMWNDERCNKKKLALCYTASCTNASCSGHGECIETINSYTCKCHPGFLGPNCEQAVTCKPQEHPDYGSLNCSHPFGPFSYNSSCSFGCKRGYLPSSMETTVRCTSSGEWSAPAPACHVVECEALTHPAHGIRKCSSNPGSYPWNTTCTFDCVEGYRRVGAQNLQCTSSGIWDNETPSCKAVT